(19) United States
(12) Patent Application Publication
(10) Pub. No.: US 2023/0192796 A1
(43) Pub. Date: Jun. 22, 2023

(54) ENGINEERED INTERLEUKIN-10 POLYPEPTIDES AND USES THEREOF

(71) Applicant: The Board of Trustees of the Leland Stanford Junior University, Stanford, CA (US)

(72) Inventors: Kenan Christopher GARCIA, Palo Alto, CA (US); Robert Andrew SAXTON, Palo Alto, CA (US)

(21) Appl. No.: 17/999,528

(22) PCT Filed: May 27, 2021
(86) PCT No.: PCT/US2021/034580

§ 371 Date: Nov. 21, 2022

Related U.S. Application Data
(60) Provisional application No. 63/031,186, filed on May 28, 2020.

(51) Int. Cl. C07K 14/54

(52) U.S. Cl. CPC C07K 14/5428; A61K 38/00

(57) ABSTRACT

The present disclosure relates generally to compositions and methods for modulating signal transduction mediated by interleukin-10 (IL-10). In particular, the disclosure provides novel IL-10 polypeptide variants with altered binding affinity to interleukin-10 receptor subunit beta (IL-10Rβ). Also provided are compositions and methods useful for producing such IL-10 polypeptide variants, as well as methods for modulating IL-10-mediated signaling, and/or for the treatment of conditions associated with the perturbation of signal transduction mediated by IL-10.

### STATEMENT REGARDING FEDERALLY SPONSORED R&D

This invention was made with Government support under contract AI51321 awarded by The National Institutes of Health. The Government has certain rights in the invention.

### CROSS-REFERENCE TO RELATED APPLICATIONS

This application claims the benefit of priority to U.S. Provisional Pat. Application Serial No. 63/031,186, filed on May 28, 2020. The disclosure of the above-referenced application is herein expressly incorporated by reference it its entirety, including any drawings.

### INCORPORATION OF THE SEQUENCE LISTING

The material in the accompanying Sequence Listing is hereby incorporated by reference into this application. The accompanying Sequence Listing text file, named 078430_520001WO_Sequence_Listing.txt, was created on May 20, 2021 and is 53 KB.

### FIELD

The present disclosure relates generally to compositions and methods for modulating signal transduction mediated by interleukin-10 (IL-10). In particular, the disclosure provides novel IL-10 polypeptide variants with altered binding affinity to interleukin-10 receptor subunit beta (IL-10Rβ). Also provided are compositions and methods useful for producing such IL-10 polypeptide variants, as well as methods for modulating IL-10-mediated signaling, and/or for the treatment of conditions associated with the perturbation of signal transduction mediated by IL-10.

### BACKGROUND

Biopharmaceuticals or the use of pharmaceutical formulations containing therapeutic protein(s) for the treatment of health conditions and diseases is a core strategy for a number of pharmaceutical and biotechnology companies. For example, several members of the cytokine family have been reported to be effective in the treatment of cancer and play a major role in the development of cancer immunotherapy. Therefore, the cytokine family has been the focus of much clinical work and effort to improve its administration and bio-assimilation.

However, the clinical success of existing therapeutic approaches involving cytokines has been limited. Their limitations are often due to off-target toxicity and ineffectiveness of the cytokines, which is largely due to the fact that cytokines have receptors on both desired and undesired responder cells that counterbalance one another and lead to unwanted side effects. In recent years, cytokine engineering has emerged as a promising strategy with various attempts to tailor cytokines to arrive at recombinant cytokines with more desired activities and reduced toxicity.

In particular, interleukin-10 (IL-10) has been considered an important anti-inflammatory cytokine that is frequently dysregulated in human diseases such as cancer and autoimmune disease. Although there has been long-standing interest in the use of recombinant IL-10 as a drug, IL-10 is highly pleiotropic, acting on many different immune cell sub-types and eliciting diverse and often opposing biological effects. In the context of autoimmune diseases, IL-10 treatment can be highly effective in mouse models due to its ability to suppress pro-inflammatory cytokine production and MHC class II expression by monocytes and macrophages. However, IL-10 also stimulates the production of the proinflammatory cytokine interferon-gamma (INFγ) by CD8+ T cells, which counteracts its anti-inflammatory effects, therefore limiting its therapeutic and clinical utility.

Hence, there is a need for additional approaches to improve properties of IL-10 for its use as a therapeutic agent. In particular, there is a need for variants of IL-10 that can selectively activate certain downstream functions and actions over others, e.g., retain many beneficial properties of IL-10 but lack its known pro-inflammatory side effects, leading to improved use of these variants as anti-tumor agents or immune modulators in treating various relevant diseases, including cancers, autoimmune diseases, and inflammatory diseases.

### SUMMARY

The present disclosure relates generally to the field of immunology, and particularly to compositions and methods for modulating signal transduction pathway mediated by interleukin 10 (IL-10) in a subject in need thereof. As described in greater detail below, without being bound by theory, IL-10-mediated signaling can be modulated via partial agonism of STAT3-mediated signaling. More particularly, in some embodiments, the disclosure provides a new class of II,-10 polypeptide variants with modulated binding affinity for the natural ligands of IL-10, e.g., interleukin 10 receptor subunit beta (IL-10Rβ). Some embodiments of the disclosure provide IL-10 partial agonists that result in a cell-type biased IL-10 signaling. Some embodiments of the disclosure provide IL-10 partial agonists that confer a cell-type biased IL-10 signaling, for example IL-10 partial agonists that display strong monocyte-biased signaling effects in human PBMCs, by suppressing monocyte and macrophage activation/cytokine production, without stimulating cytotoxic CD8 T cell functions. As discussed more in greater detail herein, these IL-10 partially agonistic variants can overcome IL-10 pleiotropy and have improved therapeutic potential. The disclosure also provides compositions and methods useful for producing such IL-10 polypeptide variants, methods for modulating IL-10-mediated signaling in a subject, as well as methods for the treatment of conditions associated with perturbations of signal transduction downstream of the receptor IL-10Rβ.

In one aspect, provided herein are recombinant polypeptides including: (a) an amino acid sequence having at least 70%, 75%, 80%, 85%, 90%, 91%, 92%, 93%, 94%, 95%, 96%, 97%, 98%, or 99% sequence identity to an interleukin-10 (IL-10) polypeptide having the amino acid sequence of SEQ ID NO: 1 or SEQ ID NO: 16; and further including (b) one or more amino acid substitution at a position corresponding to an amino acid residue selected from the group consisting of X25, X14, X18, X24, X28, X74, X90, X92, X96, X100 and X104 of SEQ ID NO: 1.

Non-limiting exemplary embodiments of the disclosed recombinant polypeptides can include one or more of the following features. In some embodiments, recombinant polypeptides include (a) an amino acid sequence having at least 70%, 75%, 80%, 85%, 90%, 91%, 92%, 93%, 94%, 95%, 96%, 97%, 98%, or 99% sequence identity to the amino acid sequence of SEQ ID NO: 1 or SEQ ID NO: 16; and (b) one or more amino acid substitution at a position corresponding to an amino acid residue selected from the group consisting of X25 and X96 of SEQ ID NO: 1 or SEQ ID NO: 16. In some embodiments, the amino acid sequence further includes at least one additional amino acid substitution at a position corresponding to an amino acid residue selected from the group consisting of X21, X22, X32, and X93 of SEQ ID NO: 1. In some embodiments, the polypeptides of the disclosure include an amino acid sequence having at least 70%, 75%, 80%, 85%, 90%, 91%, 92%, 93%, 94%, 95%, 96%, 97%, 98%, or 99% sequence identity to SEQ ID NO: 1 or SEQ ID NO: 16, and further include an amino acid substitution corresponding an amino acid residue selected from the group consisting of X25, X14, X18, X21, X22, X24, X28, X32, X74, X90, X92, X93, X96, X100 and X104 of SEQ ID NO: 1 or SEQ ID NO: 16. In some embodiments, the recombinant polypeptides have an altered binding affinity for II,-10 receptor beta (IL-10RP) compared to binding affinity of a reference IL-10 polypeptide lacking the one or more amino acid substitution. In some embodiments, the recombinant polypeptides have a reduced binding affinity for IL-10Rβ compared to binding affinity of the reference IL-10 polypeptide. In some embodiments, the recombinant polypeptides have a reduced capability to stimulate STAT3 signaling compared to the reference IL-10 polypeptide. In some embodiments, the recombinant polypeptides have an increased binding affinity for IL-10Rβ compared to binding affinity of the reference IL-10 polypeptide. In some embodiments, the recombinant polypeptides confer a cell-type biased signaling of the downstream signal transduction mediated through IL-10 compared to the reference IL-10 polypeptide.

In some embodiments, the one or more amino acid substitution is independently selected from the group consisting of an alanine substitution, an arginine substitution, an aspartic acid substitution, a histidine substitution, a glutamic acid substitution, a lysine substitution, a serine substitution, a tryptophan substitution, and combinations of any thereof.

In some embodiments, the one or more amino acid substitution is at a position corresponding to an amino acid residue selected from the group consisting of D25, H14, N18, R24, D28, E74, H90, N92, E96, T100, and R104 of SEQ ID NO: 1 or SEQ ID NO: 16. In some embodiments, the amino acid sequence further includes at least one additional amino acid substitution at a position corresponding to an amino acid residue selected from the group consisting of N21, M22, R32, and S93 of SEQ ID NO: 1 or SEQ ID NO: 16. In some embodiments, the recombinant polypeptides of the disclosure include an amino acid sequence having at least 70%, 75%, 80%, 85%, 90%, 91%, 92%, 93%, 94%, 95%, 96%, 97%, 98%, or 99% sequence identity to SEQ ID NO: 1 or SEQ ID NO: 16, and further include the amino acid substitutions corresponding to the following amino acid substitutions: (a) N18Y/N92Q/T100D/R104W; (b) N18Y/N21H/N92Q/E96D/T100V/R104W; (c) N18Y/N21H/E96H/T100V/R104W; (d) N18Y/D25A/N92Q/T100D/R104W; (e) N18Y/D25K/N92Q/T100D/R104W; and (f) N18Y/D25A/N92Q/E96A/T100D/R104W. In some embodiments, recombinant polypeptides of the disclosure include an amino acid sequence having at least 70%, 75%, 80%, 85%, 90%, 91%, 92%, 93%, 94%, 95%, 96%, 97%, 98%, or 99% sequence identity to SEQ ID NO: 1 or SEQ ID NO: 16, and further include the amino acid substitutions corresponding to the following amino acid substitutions: (a) D25A; (b) D25K; (c) E96A; (d) E96K; (e) D25A/E96A; (f) N21A/R104A; (g) N21A/D25A; (h) N21A/D25A/E96A; and (i) N21A/M22A/D25A.

In some embodiments, the polypeptides of the disclosure include an amino acid sequence having at least 70%, 75%, 80%, 85%, 90%, 91%, 92%, 93%, 94%, 95%, 96%, 97%, 98%, or 99% sequence identity to SEQ ID NO: 1 or SEQ ID NO: 16, and further include an amino acid substitution corresponding an amino acid residue selected from the group consisting of D25, H14, N18, N21, M22, R24, D28, R32, E74, H90, N92, S93, E96, T100 and R104 of SEQ ID NO: 1 or SEQ ID NO: 16.

In one aspect, some embodiments of the disclosure relate to recombinant nucleic acid molecules, wherein the nucleic acids include a nucleic acid sequence encoding a polypeptide that includes an amino acid sequence having at least 90% sequence identity to the amino acid sequence of the polypeptide of the disclosure. In some embodiments, the nucleic acid sequence is operably linked to a heterologous nucleic acid sequence. In some embodiments, the nucleic acid molecule is incorporated into an expression cassette or an expression vector

In another aspect, some embodiments of the disclosure relate to recombinant cells, wherein the recombinant cells include: (a) a recombinant polypeptide of the disclosure; and/or (b) a recombinant nucleic acid of the disclosure. In some embodiments, the recombinant cell is a eukaryotic cell. In some embodiments, the eukaryotic cell is a mammalian (e.g., human) cell. In a related aspect, some embodiments of the disclosure relate to cell cultures including at least one recombinant cell of the disclosure and a culture medium.

In another aspect, some embodiments of the disclosure relate to methods for producing a polypeptide, wherein the methods include: (a) providing one or more recombinant cells of the disclosure; and (b) culturing the one or more recombinant cells in a culture medium such that the cells produce the polypeptide encoded by the recombinant nucleic acid molecule. In some embodiments, the methods further include isolating and/or purifying the produced polypeptide. In some embodiments, the methods further structurally modifying the produced polypeptide to increase half-life. In some embodiments, the modification includes one or more alterations selected from the group consisting of fusion to a human Fc antibody fragment, fusion to albumin, and PEGylation.

In another aspect, some embodiments of the disclosure relate to pharmaceutical compositions, wherein the pharmaceutical compositions include a pharmaceutically acceptable carrier and one or more of: (a) a recombinant polypeptide of the disclosure; (b) a recombinant nucleic acid of the disclosure; and (c) a recombinant cell of the disclosure. In some embodiments, the composition includes a recombinant polypeptide of the disclosure and a pharmaceutically acceptable carrier. In some embodiments, the composition comprises a receombinant viral vector comprising a nucleic acid sequence encoding the polypeptide of the disclosure and a pharmaceutically acceptable carrier. In some embodiments, the composition includes a recombinant cell comprising a nucleic acid encoding a polypeptide of the disclosure and a pharmaceutically acceptable carrier.

In one aspect, some embodiments of the disclosure relate to methods for modulating IL-10-mediated signaling in a subject, wherein the methods include administering to the subject a composition including one or more of: (a) a recombinant polypeptide of the disclosure; (b) a recombinant nucleic acid of the disclosure; (c) a recombinant cell of the disclosure; (d) a recombinant viral or non-viral vector comprising a nucleic acid of the disclosure; and (e) a pharmaceutical composition of the disclosure.

In another aspect, some embodiments of the disclosure relate to methods for the treatment of a health condition in a subject in need thereof, wherein the methods includes administering to the subject a composition including one or more of: (a) a recombinant polypeptide of the disclosure; (b) a recombinant nucleic acid of the disclosure; (c) a recombinant cell of the disclosure; (d) a recombinant viral or non-viral vector comprising a nucleic acid of the disclosure; and (e) a pharmaceutical composition of the disclosure. In some embodiments, the administered composition results in a cell-type biased signaling of the downstream signal transduction mediated through IL-10 compared to a reference IL-10 polypeptide lacking the one or more amino acid substitution. In some embodiments, the cell-type biased IL-10 signaling comprises a reduction of STAT1- or STAT3-mediated pro-inflammatory function in B cells, T cells, and NK cells while substantially retaining its STAT3-mediated anti-inflammatory function in monocytes and macrophages. In some embodiments, the STAT3-mediated signaling is determined by an assay selected from the group consisting of by a gene expression assay, a phospho-flow signaling assay, and an enzyme-linked immunosorbent assay (ELISA). In some embodiments, the STAT3-mediated pro-inflammatory function is selected from the group consisting of cytokine production, chemokine production, immune cell proliferation, and immune cell recruitment. In some embodiments, the STAT3-mediated pro-inflammatory function is reduced from about 20% to about 100%. In some embodiments, the administered composition results in a reduced capacity to induce expression of a pro-inflammatory gene selected from IFN-y, granzyme B, granzyme A, perforin, TNF-a, GM-CSF, and MIP1α in the subject. In some embodiments, the administered composition stimulates expression of interferon gamma (INFy) in CD8+ T cells. In some embodiments, health condition is a cancer. In some embodiments, the health condition is an autoimmune disease. In some embodiments, the health condition is a chronic infection.

In another aspect, some embodiments of the disclosure relate to kits for modulating IL-10-mediated signaling in a subject, or for treating a health condition in a subject in need thereof, wherein the kits include one or more of: (a) a recombinant polypeptide of the disclosure; (b) a recombinant nucleic acid of the disclosure; (c) a recombinant cell of the disclosure; and (d) a pharmaceutical composition of the disclosure.

The foregoing summary is illustrative only and is not intended to be in any way limiting. In addition to the illustrative embodiments and features described herein, further aspects, embodiments, objects and features of the disclosure will become fully apparent from the drawings and the detailed description and the claims.

### BRIEF DESCRIPTION OF THE DRAWINGS

FIGS. 1A-1B depict Top view (1A) and side view (1B) of the molecular model of the IL-10 receptor complex with IL-10, IL- 10Rα, and IL-l0Rβ.

FIGS. 2A-2F schematically summarize the results of experiments illustrating the structural basis for receptor engagement by IL-10. FIGS. 2A-2B: Two views of the IL-10/IL-10Rα/IL-10Rβ ternary sub-complex, with IL-10, IL- 10Rα, and IL-10Rβ. FIGS. 2C-2E: Close-up views of the IL-10/IL-10Rβ binding interface. Hydrogen bonds and salt-bridges are shown as black dashed-lines. Mutated residues in affinity matured IL-10 (super-10) are italicized. FIG. 2F: Mutations at the IL-10RP interface reduce IL-10 signaling activity. Dose response curves for phospho-Y705-STAT3 in Daudi cells stimulated with WT IL-10 or indicated variants for 20 minutes and analyzed by flow cytometry. Data are mean +/- SD for two independent replicates, shown as a percent of maximal WT IL-10 signal.

FIGS. 3A-3E schematically summarize the results of experiments illustrating that tuning IL-10Rβ affinity reveals differential IL-10 signaling plasticity across cell types. FIG. 3A: IL-10Rβ binding mutants show differential signaling responses in Daudi and THP-1 cells. Cell lines were treated with 100 nM IL-10 or indicated mutant for 20 minutes and analyzed by flow cytometry. Data are mean +/- SD for two independent replicates. FIG. 3B: View of IL-10 helices α1 and α3, showing mutated residues in high-affinity super-10 and partial agonist 10-DE (D25A/E96A double mutant). FIG. 3C: Normalized Eₘₐₓ values for phospho-Y705-STAT3 calculated from sigmoidal dose-response curves shown in FIG. 3D. Data are mean +/- SD for three replicates. FIG. 3D: L-10 mutants Super-10″ and “10-DE” exhibit differential signaling profiles across cell types. Dose response curves for phospho-Y705-STAT3 in the indicated cell lines stimulated with WT IL-10, Super-10, D25A/E96A mutant (10-DE) for 20 minutes and analyzed by flow cytometry. Data are mean +/- SD for three replicates, shown as a percent of maximal WT IL-10 signal in that cell line. FIG. 3E: IL-10 signaling plasticity correlates with IL10Rβ expression. Histograms showing fluorescent intensity of surface stained IL- 10Rα (left) and IL-10RP (right) on the indicated cell lines analyzed by flow cytometry.

FIGS. 4A-4G schematically summarize the results of experiments illustrating that an exemplary myeloid-biased partial agonist uncouples anti-inflammatory and immunostimulatory functions of IL-10. FIG. 4A: IL-10RP is differentially expressed in primary human immune cells. Histograms showing fluorescent intensity of IL-10RP on the indicated cell types in human PBMCs analyzed by flow cytometry, using gating strategy as shown in FIG. 6A. FIG. 4B: IL-10 variant 10-DE elicits monocyte-biased signaling in human PBMCs. Normalized phospho- Y705-STAT3 signaling response in the indicated cell types in human PBMCs treated with 10 nM WT IL-10 or indicated variant analyzed by flow cytometry. Data are mean +/- SD for two replicates, and is a representative experiment performed three times with different donor PBMCs. FIG. 4C: 10-DE suppresses inflammatory cytokine production in bulk PBMCs. PBMCs were stimulated with 1 nM LPS alone or with the addition of 10 nM WT IL-10, 10-DE, or Super-10 for 24 hours. Levels of TNF-a, IL-6, and IL-8 in the supernatant were analyzed by ELISA. Data are mean +/- SEM for three independent replicates. FIG. 4D: 10-DE downregulates MHC-II surface expression on activated monocytes. PBMCs were stimulated with 1 nM LPS and PBS, 10 nM WT IL-10, 10-DE, or Super-10 for 24 hours, and monocytes (CD 14+) were analyzed for surface HLA-DR expression by flow cytometry. FIG. 4E: 10-DE suppresses inflammatory cytokine production in human monocyte-derived macrophages. Isolated human macrophages were stimulated with 1 nM LPS alone or with the addition of 10 nM WT IL-10, 10-DE, or Super-10 for 24 hours. Levels of TNF-a, IL-6, and IL-8 in the supernatant were analyzed by ELISA. Data are mean +/- SEM for three independent replicates. FIG. 4F: 10-DE does not potentiate IFN-γ or Granzyme B production by CD8+ T cells. Pre-activated CD8+ T cells from human PBMCs were stimulated with anti-CD3 antibody alone or in combination with 10 nM WT IL-10, 10-DE, or super-10 for 3 hours. Levels of IFN-γ and Granzyme B in the supernatant were measured by ELISA. Data are mean +/- SEM for three independent replicates. FIG. 4G: Model showing how cell-type biased signaling of IL-10 variant 10-DE uncouples the immunosuppressive and immunostimulatory functions of IL-10.

FIG. 5A: Table showing the seven residues targeted for randomization, the codons used for each site in library generation, and the amino acids present at each location in clones 5.1, 5.2, and 5.3 (SEQ ID NOS: 2, 3, and 4 respectively). FIG. 5B: Affinity matured IL-10 variants show enhanced binding to IL-10Rβ. Binding titration of SA-647-IL-10Rβ on yeast displaying WT monomeric IL-10 or affinity matured clones. Yeast were pre-bound with 500 nM unlabeled IL-10Rα. FIGS. 5C-5D: Surface plasmon resonance sensogram of soluble IL-10RP binding to immobilized affinity matured IL-10 clone 5.1 either alone (FIG. 5F) or pre-bound with saturating soluble IL-10Rα (FIG. 5D). K_{D}, dissociation constant.

FIG. 6A: Gating strategy for PBMC phospho-flow cytometry signaling experiments in FIGS. 4A-4B. FIG. 6B: Dose response curves for phospho-Y705-STAT3 in human PBMCs stimulated with WT IL-10 or 10-DE for 20 minutes and analyzed by flow cytometry. Data are mean +/- SD for two replicates, shown as a percent of maximal WT II,-10 signal. Shown is a representative experiment performed at least three times with different donor PBMCs.

FIG. 7A: Levels of IL-1β from bulk human PBMCs treated with LPS and the indicated IL-10 variant, measured by ELISA (mean ± SEM, n=3, N=2, **P<0.01, two-sided Student’s t test). FIGS. 7B-7C: Surface HLA-DR (7B) and CD86 surface expression (7C) on monocytes activated with LPS alone or in combination with 10 nM of the indicated IL-10 variant, analyzed by flow cytometry (mean ± SD, n=4, N=2). FIG. 7D: Levels of IFN-γ from bulk PBMCs stimulated with anti-CD3 antibody for 72 hours, alone or in combination with the indicated IL-10 variants, measured by ELISA. (mean ± SEM, n=3, N=3, **P<0.01, two-sided Student’s t test). FIG. 7E: Survival after intraperitoneal injection of LPS (15 mg/kg) in combination with PBS, WT IL-10 or 10-DE (n=8 mice, N=2, **P <0.01, log-rank Mantel-Cox test). FIGS. 7F-7G: Levels of TNF-a, IL-6, and haptoglobin in mouse serum following injection of LPS (4 mg/kg) and the indicated IL-10 variant, measured by ELISA (mean ± SEM, n=3, N=2, *P<0.05, **P<0.01, two-sided Student’s ttest).

FIG. 8A: Phospho-STAT1 activation in primary immune cells treated with 10 nM of WT or mutant IL-10, analyzed by flow cytometry. (mean ± SEM, n=3, N=2). FIG. 8B: Normalized MFI values for phospho-STAT3 from CD4⁺ T cells treated with 10 nM WT IL-10, Super-10, or 10-DE and analyzed by flow cytometry (mean ± SEM, n=4, N=2). FIG. 8C: Fold change of select IL-10-regulated genes in activated CD8⁺ T cells treated with the indicated IL-10 variants for 24 hours, analyzed by RNA-seq (n=2 biological replicates).

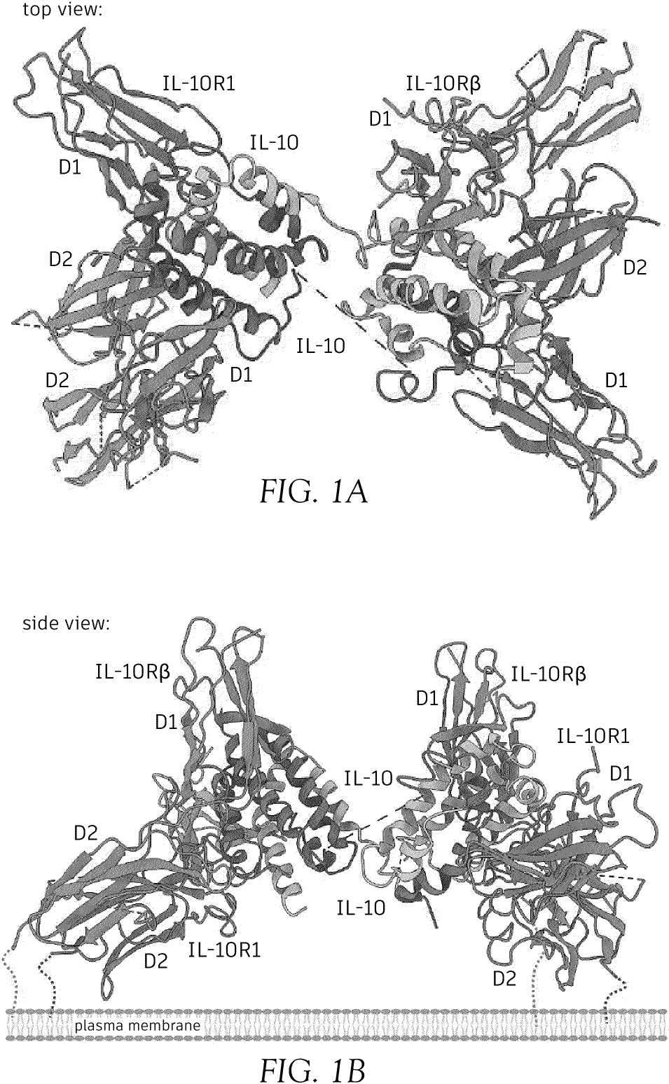

### DETAILED DESCRIPTION OF THE DISCLOSURE

The present disclosure generally relates to compositions and methods for selectively modulating signal transduction pathway mediated by interleukin 10 (IL-10) in a subject. As described in greater detail below, IL-10-mediated signaling can be modulated via cell-type biased agonism of STAT3-mediated signaling. Some embodiments of the disclosure provide novel IL-10 polypeptides which are designed based on new insights into how IL-10 interacts with its cognate receptors, in particular, IL-10RJ3. Some embodiments provide a class of IL-10 polypeptide variants with modulated binding affinity for the natural receptors of IL-10, e.g., interleukin 10 receptor subunit beta (IL-10Rβ). Some embodiments of the disclosure provide IL-10 partial agonists that possess a cell-type selective IL-10 signaling, for example conferring a reduction in a STAT3-mediated pro-inflammatory function in bulk PBMCs, B cells, T cells, and NK cells while substantially (e.g., 90-110%) retains its STAT3-mediated function in monocytes and macrophages. The disclosure also provides compositions and methods useful for producing such IL-10 polypeptide variants, methods for modulating IL-10-mediated signaling in a subject, as well as methods for the treatment of conditions associated with perturbations of signal transduction downstream of the IL-10 receptor.

As described in greater detail below, a structure-based approach was used to deconvolute IL-10 actions by determining the cryo-EM structure of the complete IL-10 receptor signaling complex, which in turns enables design of myeloid-selective agonists. A star-shaped hexameric structure of the IL-10 receptor signaling complex shows how IL-10 and IL-10Rα form a composite surface to engage the shared receptor subunit IL-10RP, enabling the design of IL-10 partial agonists. Characterization of a series of IL-10 mutants designed with a range of IL-1OR0-binding affinities revealed how IL-10 signaling plasticity varies across cell types. In particular, the experimental data presented herein revealed that mutations in the IL-10 binding site for IL-10Rβ result in biased agonists capable of selectively suppress pro-inflammatory function and/or STAT3-mediated signaling in a cell-type dependent manner. Certain IL-10 biased agonists disclosed herein can confer a reduction of STAT3-mediated function in bulk PBMCs, B cells, T cells, and NK cells while substantially retains STAT3-mediated function in monocytes and macrophages. As an exemplification of such IL-10 biased agonists, the experimental results presented in Examples 5-6 describe an IL-10 variant having a myeloid-biased activity that includes suppressing inflammatory macrophage activation without promoting interferon-y production.

These results provide insights into the mechanisms underlying IL-10 pleiotropy and demonstrate how IL-10 signaling plasticity can be exploited to tune II,-10 function.

### Definitions

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. Many of the techniques and procedures described or referenced herein are well understood and commonly employed using conventional methodology by those skilled in the art.

The singular form “a”, “an”, and “the” include plural references unless the context clearly dictates otherwise. For example, the term “a cell” includes one or more cells, comprising mixtures thereof. “A and/or B” is used herein to include all of the following alternatives: “A”, “B”, “A or B”, and “A and B”.

The term “about”, as used herein, has its ordinary meaning of approximately. If the degree of approximation is not otherwise clear from the context, “about” means either within plus or minus 10% of the provided value, or rounded to the nearest significant figure, in all cases inclusive of the provided value. Where ranges are provided, they are inclusive of the boundary values.

The terms “administration” and “administering”, as used herein, refer to the delivery of a bioactive composition or formulation by an administration route comprising, but not limited to, oral, intravenous, intra-arterial, intramuscular, intraperitoneal, subcutaneous, intramuscular, and topical administration, or combinations thereof. The term includes, but is not limited to, administering by a medical professional and self-administering.

The terms “cell”, “cell culture”, and “cell line” refer not only to the particular subject cell, cell culture, or cell line but also to the progeny or potential progeny of such a cell, cell culture, or cell line, without regard to the number of transfers or passages in culture. It should be understood that not all progeny are exactly identical to the parental cell. This is because certain modifications may occur in succeeding generations due to either mutation (e.g., deliberate or inadvertent mutations) or environmental influences (e.g., methylation or other epigenetic modifications), such that progeny may not, in fact, be identical to the parent cell, but are still included within the scope of the term as used herein, so long as the progeny retain the same functionality as that of the original cell, cell culture, or cell line.

The term “effective amount”, “therapeutically effective amount”, or “pharmaceutically effective amount” of a subject recombinant polypeptide of the disclosure generally refers to an amount sufficient for a composition to accomplish a stated purpose relative to the absence of the composition (e.g., achieve the effect for which it is administered, treat a disease, reduce a signaling pathway, or reduce one or more symptoms of a disease or health condition). An example of an “effective amount” is an amount sufficient to contribute to the treatment, prevention, or reduction of a symptom or symptoms of a disease, which could also be referred to as a “therapeutically effective amount.” A “reduction” of a symptom means decreasing of the severity or frequency of the symptom(s), or elimination of the symptom(s). The exact amount of a composition including a “therapeutically effective amount” will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

As used herein, the term “variant” of an IL-10 polypeptide refers to a polypeptide in which one or more amino acid substitutions, deletions, and/ or insertions are present as compared to the amino acid sequence of a reference IL-10 polypeptide, e.g., a wild-type IL-10 polypeptide. As such, the term “IL-10 polypeptide variant” includes naturally occurring allelic variants or alternative splice variants of an IL-10 polypeptide. For example, a polypeptide variant includes the substitution of one or more amino acids in the amino acid sequence of a parent IL-10 polypeptide with a similar or homologous amino acid(s) or a dissimilar amino acid(s).

The term “operably linked”, as used herein, denotes a physical or functional linkage between two or more elements, e.g., polypeptide sequences or polynucleotide sequences, which permits them to operate in their intended fashion. For example, an operably linkage between a polynucleotide of interest and a regulatory sequence (for example, a promoter) is functional link that allows for expression of the polynucleotide of interest. It should be understood that, operably linked elements may be contiguous or non-contiguous. In the context of a polypeptide, “operably linked” refers to a physical linkage (e.g., directly or indirectly linked) between amino acid sequences (e.g., different domains) to provide for a described activity of the polypeptide. In the present disclosure, various domains of the recombinant polypeptides of the disclosure may be operably linked to retain proper folding, processing, targeting, expression, binding, and other functional properties of the recombinant polypeptides in the cell. Operably linked domains of the recombinant polypeptides of the disclosure may be contiguous or non-contiguous (e.g., linked to one another through a linker).

The term “percent identity,” as used herein in the context of two or more nucleic acids or proteins, refers to two or more sequences or subsequences that are the same or have a specified percentage of nucleotides or amino acids that are the same (e.g., about 60% sequence identity, 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection. See e.g., the NCBI web site at ncbi.nlm.nih.gov/BLAST. Such sequences are then said to be “substantially identical.” This definition also refers to, or may be applied to, the complement of a sequence. This definition also includes sequences that have deletions and/or additions, as well as those that have substitutions. Sequence identity can be calculated using published techniques and widely available computer programs, such as the GCS program package (Devereux et al, Nucleic Acids Res. 12:387, 1984), BLASTP, BLASTN, FASTA (Atschul et al., J Mol Biol 215:403, 1990). Sequence identity can be measured using sequence analysis software such as the Sequence Analysis Software Package of the Genetics Computer Group at the University of Wisconsin Biotechnology Center (1710 University Avenue, Madison, Wis. 53705), with the default parameters thereof.

The term “pharmaceutically acceptable excipient” as used herein refers to any suitable substance that provides a pharmaceutically acceptable carrier, additive or diluent for administration of a compound(s) of interest to a subject. As such, “pharmaceutically acceptable excipient” can encompass substances referred to as pharmaceutically acceptable diluents, pharmaceutically acceptable additives, and pharmaceutically acceptable carriers. As used herein, the term “pharmaceutically acceptable carrier” includes, but is not limited to, saline, solvents, dispersion media, coatings, antibacterial and antifungal agents, isotonic and absorption delaying agents, and the like, compatible with pharmaceutical administration. Supplementary active compounds (e.g., antibiotics and additional therapeutic agents) can also be incorporated into the compositions.

The term “recombinant” or “engineered” nucleic acid molecule or polypeptide as used herein, refers to a nucleic acid molecule or polypeptide that has been altered through human intervention. As non-limiting examples, a cDNA is a recombinant DNA molecule, as is any nucleic acid molecule that has been generated by in vitro polymerase reaction(s), or to which linkers have been attached, or that has been integrated into a vector, such as a cloning vector or expression vector. As non-limiting examples, a recombinant nucleic acid molecule can be one which: 1) has been synthesized or modified in vitro, for example, using chemical or enzymatic techniques; 2) includes conjoined nucleotide sequences that are not conjoined in nature; 3) has been engineered using molecular cloning techniques such that it lacks one or more nucleotides with respect to the naturally occurring nucleic acid molecule sequence; and/or 4) has been manipulated using molecular cloning techniques such that it has one or more sequence changes or rearrangements with respect to the naturally occurring nucleic acid sequence. As non-limiting examples, a cDNA is a recombinant DNA molecule, as is any nucleic acid molecule that has been generated by in vitro polymerase reaction(s), or to which linkers have been attached, or that has been integrated into a vector, such as a cloning vector or expression vector. Another non-limiting example of a recombinant nucleic acid and recombinant protein is an II,-10 polypeptide variant as disclosed herein.

As used herein, an “individual” or a “subject” includes animals, such as human (e.g., human individuals) and non-human animals. In some embodiments, an “individual” or “subject” is a patient under the care of a physician. Thus, the subject can be a human patient or an individual who has, is at risk of having, or is suspected of having a disease of interest (e.g., cancer) and/or one or more symptoms of the disease. The subject can also be an individual who is diagnosed with a risk of the condition of interest at the time of diagnosis or later. The term “non-human animals” includes all vertebrates, e.g., mammals, e.g., rodents, e.g., mice, non-human primates, and other mammals, such as e.g., sheep, dogs, cows, chickens, and non-mammals, such as amphibians, reptiles, etc.

The term “vector” is used herein to refer to a nucleic acid molecule or sequence capable of transferring or transporting another nucleic acid molecule. The transferred nucleic acid molecule is generally linked to, e.g., inserted into, the vector nucleic acid molecule. Generally, a vector is capable of replication when associated with the proper control elements. The term “vector” includes cloning vectors and expression vectors, as well as viral vectors and integrating vectors. An “expression vector” is a vector that includes a regulatory region, thereby capable of expressing DNA sequences and fragments in vitro and/or in vivo. A vector may include sequences that direct autonomous replication in a cell, or may include sequences sufficient to allow integration into host cell DNA. Useful vectors include, for example, plasmids (e.g., DNA plasmids or RNA plasmids), transposons, cosmids, bacterial artificial chromosomes, and viral vectors. Useful viral vectors include, e.g., replication defective retroviruses and lentiviruses. In some embodiments, a vector is a gene delivery vector. In some embodiments, a vector is used as a gene delivery vehicle to transfer a gene into a cell.

It is understood that aspects and embodiments of the disclosure described herein include “comprising”, “consisting”, and “consisting essentially of′ aspects and embodiments.

As used herein, “comprising” is synonymous with “including”, “containing”, or “characterized by”, and is inclusive or open-ended and does not exclude additional, unrecited elements or method steps. As used herein, “consisting of′ excludes any elements, steps, or ingredients not specified in the claimed composition or method. As used herein, “consisting essentially of′ does not exclude materials or steps that do not materially affect the basic and novel characteristics of the claimed composition or method. Any recitation herein of the term “comprising”, particularly in a description of components of a composition or in a description of steps of a method, is understood to encompass those compositions and methods consisting essentially of and consisting of the recited components or steps.

Headings, e.g., (a), (b), (i) etc., are presented merely for ease of reading the specification and claims. The use of headings in the specification or claims does not require the steps or elements be performed in alphabetical or numerical order or the order in which they are presented.

As will be understood by one having ordinary skill in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to”, “at least”, “greater than”, “less than”, and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub- combination was individually and explicitly disclosed herein.

### Interleukin-10 and Inflammatory Immune Responses

The cytokine interleukin-10 (IL-10) is an immunoregulatory cytokine that possesses both anti-inflammatory and immunostimulatory properties and is frequently dysregulated in human disease. It is a pleiotropic cytokine expressed as a non-covalently linked homodimer of ~37 kDa and regulates multiple immune responses through actions on T cells, B cells, macrophages, and antigen presenting cells (APC). Its predominantly anti-inflammatory properties have been widely reported. II,-10 has been reported to suppress immune responses by inhibiting expression of IL-1, IL-10, IL-6, IL-8, TNF-a, GM-CSF, and G-CSF in activated monocytes and activated macrophages. Although IL-10 is predominantly expressed in macrophages, expression has also been detected in activated T cells, B cells, mast cells, and monocytes. In addition to suppressing immune responses, IL-10 exhibits immuno-stimulatory properties, including stimulating the proliferation of thymocytes treated with IL-2 and IL-4, enhancing the viability of B cells, and stimulating the expression of MHC class II.

Various immuno-stimulatory properties of IL-10 have been reported. IL-10 can costimulate B-cell activation, prolong B-cell survival, and contribute to class switching in B-cells. Moreover, it can costimulate natural killer (NK) cell proliferation and cytokine production and act as a growth factor to stimulate the proliferation of certain subsets of CD8+ T cells. It has been reported that high doses of IL-10 in humans can lead to an increased production of INFγ. IL-10 signals through a two-receptor complex consisting of two copies each of IL-10 receptor 1 (IL-lORa) and IL-10RJ3. It has been reported that IL-10Rα binds IL-10 with a relatively high affinity (-35-200 pM), and the recruitment of IL-10Rβ to the receptor complex makes only a marginal contribution to ligand binding. However, the engagement of IL-10Rβ to the complex enables signal transduction following ligand binding. Thus, the functional receptor consists of a dimer of heterodimers of IL-10Rα and IL-10RJ3. Most hematopoietic cells constitutively express low levels of IL-10Rα, and receptor expression can often be dramatically upregulated by various stimuli. In contrast, the IL-10Rβ is expressed on most cells. The binding of IL-10 to the receptor complex activates the Janus tyrosine kinases, JAK1 and Tyk2, associated with IL-10Rα and IL-10Rβ, respectively, to phosphorylate the cytoplasmic tails of the receptors. This results in the recruitment of STAT3 to the IL-10Rα. The homodimerization of STAT3 results in its release from the receptor and translocation of the phosphorylated STAT homodimer into the nucleus, where it binds to STAT3-binding elements in the promoters of various genes. One of these genes is IL-10 itself, which is positively regulated by STAT3. STAT3 also activates the suppressor of cytokine signaling 3 (SOCS3), which controls the quality and quantity of STAT activation. SOCS3 is induced by IL-10 and exerts negative regulatory effects on various cytokine genes.

As a result of its pleiotropic activity, IL-10 has been linked to a broad range of diseases, disorders and conditions, including inflammatory conditions, immune -related disorders, fibrotic disorders and cancer. In view of the prevalence and severity of IL-10 -associated diseases, disorders and conditions, novel IL-10 agents and modifications thereof would be of tremendous value in the treatment and prevention of IL-10 - associated diseases, disorders and conditions.

The IL-10 receptor signaling complex consists of alpha and beta subunits, which are also referred to as IL-10R1 and IL-10R2, or IL-10Rα and IL-10RJ3, respectively. Receptor activation requires binding to both alpha and beta. As described in greater detail below, a structure-based approach was used to deconvolute IL-10 actions, e.g., pleiotropy, by determining the 3.5-A cryo-EM structure of the IL-10 receptor signaling complex. The stelliform (e.g., star-shaped) hexameric structure described herein shows how IL-10 and IL-10Rα form a composite surface to engage the shared receptor subunit IL-10RP, enabling the design of IL-10 partial agonists. As described in greater detail below, characterizing a series of designed IL-10 variants with a range of IL-10RP-binding affinities (e.g., strengths) revealed how IL-10 signaling plasticity varies across cell types, uncovered substantial differences in signaling and gene expression response thresholds across immune cell populations, which in turn provides a means of manipulating IL-10 cell-type selectivity. Some variants were found to display myeloid-biased activity; suppressing monocyte and macrophage activation without stimulating inflammatory CD8⁺ T cell functions, thereby uncoupling the major opposing functions of IL-10. These results provide insights into the mechanisms underlying IL-10 pleiotropy and demonstrate how IL-10 signaling plasticity can be exploited to tune IL-10 function and further provide a structural and mechanistic blueprint for tuning the pleiotropic actions of IL-10.

The term IL-10 or IL-10 polypeptide refers to wild-type IL-10, whether native or recombinant, and encompasses homologs, orthologs, variants, and fragments thereof, as well as IL-10 polypeptides having, for example, a leader sequence (e.g., a signal peptide). As such, an IL-10 polypeptide includes, but not limited to, a recombinantly produced IL-10 polypeptide, synthetically produced IL-10 polypeptide, and IL-10 polypeptide extracted from cells or tissues. As a non-limiting example of IL-10 polypeptides of the disclosure, an amino acid sequence of mature human IL-10 is depicted in SEQ ID NO: 1. Exemplary IL-10 homologs and modified forms thereof from other mammalian species include IL-10 polypeptides from rat (accession NP_036986.2; GI 148747382); cow (accession NP_776513.1; GI 41386772); sheep (accession NP_OO 1009327.1; GI 57164347); dog (accession ABY86619.1; GI 166244598); and rabbit (accession AAC23839.1; GI 3242896). Further examples of IL-10 polypeptides suitable for introduction of amino acid substitutions described herein include, but are not limited to, virus-encoded IL-10 homologs, including IL-10 polypeptides from genera Cytomegalovirus, Lymphocryptovirus, Macavirus, Percavirus, Parapoxvirus, Capripoxvirus, and Avipoxvirus. Non-limiting examples of cytomegalovirus IL-10 polypeptides include those from human cytomegalovirus (accession AAR31656 and ACR49217), Green monkey cytomegalovirus (accession AEV80459), rhesus cytomegalovirus (accession AAF59907), baboon cytomegalovirus (accession AAF63436), owl monkey cytomegalovirus (accession AEV80800), and squirrel monkey ytomegalovirus (accession AEV80955). Examples of lymphocryptovirus IL-10 polypeptides include those from Epstein-Barr virus (accession CAD53385), Bonobo herpesvirus (accession XP_003804206.1), Rhesus lymphocryptovirus (accession AAK95412), and baboon lymphocryptovirus (accession AAF23949). Additional information regarding viral IL-10 polypeptides and their control of host immune function can be found in, for example, Slobedman B. et al., J. Virol. October 2009, p. 9618-9629; and Ouyang P. et al., J. Gen. Virol. (2014), 95, 245-262.

An amino acid sequence of wild-type human IL-10 precursor polypeptide (e.g., pre-protein with a signal peptide) is depicted in SEQ ID NO: 16, which is a 178 amino acid residue protein with an N-terminal 18 amino acid signal peptide that can be removed to generate a 160 amino acid mature protein of SEQ ID NO: 1. However, mature proteins are often used to generate recombinant polypeptide constructs. Therefore, for the purpose of the present disclosure, all amino acid numbering is based on the mature polypeptide sequence of the IL-10 protein set forth in SEQ ID NO: 1.

Inflammation is essential for protecting organisms against infection and promoting tissue homeostasis. However, excessive immune cell activation can damage bystander tissues and result in organ malfunction, chronic inflammation, and autoimmune disease. IL-10 is an important anti-inflammatory cytokine that plays a central role in regulating and terminating inflammatory responses. In some instances, IL-10 is dysregulated in autoimmune disease, cancer, and chronic infection. A variety of immune cells produce IL-10 during inflammation, which in turn exerts potent anti-inflammatory effects primarily by inhibiting cytokine production and antigen presentation by activated myeloid cells. Consistent with this role, genetic loss of either IL-10 or the IL-10 receptor results in severe inflammation and autoimmune disease, such as inflammatory bowel disease (IBD), in both mice and humans. In addition, dysregulated IL-10 function has also been associated with cancer and chronic inflammation.

Despite these important anti-inflammatory functions, IL-10 is also highly pleiotropic, eliciting diverse and seemingly opposed biological effects. Most notably, IL-10 also potentiates TCR-stimulated CD8+ T cell activity, enhancing production of the pro-inflammatory cytokine interferon-γ (IFN-₇) as well as cytolytic factors such as granzyme B. Indeed, administration of exogenous IL-10 has been shown to elevate serum IFN-γ levels in human clinical trials, possibly counteracting its anti-inflammatory effects.

IL-10 functions as a secreted homodimer that initiates signaling in receiver cells by engaging two copies of a heterodimeric receptor complex consisting of a high affinity, private receptor subunit, IL-10Ra, and a low-affinity, shared receptor subunit, IL,-10Rβ. IL-10 drives the dimerization of IL-10Rα and IL-1OR0, resulting in the phosphorylation and activation of the transcription factor STAT3 (signal transducer and activator of transcription 3) and to a lesser extent STAT1, which mediate the diverse functional effects of IL-10.

Functionally selective, or “biased”, agonists that uncouple downstream signaling responses have been extensively characterized for G-protein coupled receptors (GPCRs), and recent studies suggest that such agonists are possible for cytokine receptors as well. However, these approaches rely on extensive structural information, which is currently lacking for the IL-10 receptor complex. There has been a lack of structural information for the complete IL-10 receptor signaling complex, including IL-1OR0, which would enable interrogation of this axis with designed variants. This lack of structural information is due principally to the extremely low affinity of IL-10 for its shared receptor subunit, IL-10Rβ, which hinders complex assembly in vitro.

As described in the Examples below, a directed evolution approach has been used to engineer an IL-10 variant with enhanced affinity for IL-10RP, enabling us to solve the cryo-EM structure of the hexameric IL-10/IL10R1/IL-10RP complex to 3.5 Å resolution. Structure-guided mutations in IL-10 targeting the IL-10RP interface resulted in biased IL-10 variants that elicited cell-type selective signaling responses in vivo. In particular, IL-10 variant 10-DE is capable of eliciting reduced IL-10 signaling in B cells, T cells, and NK cells while substantially retaining IL-10 signaling in monocytes and macrophages, thereby uncoupling the immunosuppressive and immunostimulatory functions of IL-10 in vivo.

Cytokines play numerous important roles in controlling host immune responses and promoting tissue homeostasis, but often also exert pleiotropic or counterproductive effects that can limit their use as therapeutics. As a result, while cytokine and cytokine receptor antagonists have achieved significant clinical success, there are far fewer such examples for cytokine receptor agonists. The cytokine IL-10 is a prime example of a cytokine that can exert both beneficial and deleterious functions depending on the tissue and disease context. As described in the greater detail below, a structure-guided approach has been used to develop functionally selective variants of II,-10 that effectively uncouple the immunosuppressive and immunostimulatory functions of IL-10, revealing new insights into how IL-10 exerts these distinct functions, while also providing a path for the development of improved cytokine-based therapeutics.

The primary barrier preventing previous structural characterization of the IL-10 receptor complex was the presence of extremely low affinity interactions between subunits, specifically IL-10 and IL-10RJ3. This barrier has been overcome by using a ligand engineering approach that allowed for obtaining access to the ternary receptor complex.

As described in greater detail below, analyzing the IL-10/IL-10Rβ binding site enabled the generation of a series of IL-10 variants with amino acid substitutions at the IL-10Rβ binding interface of IL-10 that resulted in cell-type biased agonism. Although previous examples of engineered cytokine receptor ligands with biased activity have relied on altering receptor topology, the experimental results described below suggest that modulating the affinity of a natural cytokine for its receptor can also be sufficient to generate biased agonism.

Without being bound to any particular theory, the experimental data described herein supports a model in which a combination of affinity for IL-10Rβ and level of IL-10Rβ expression determines the type of signaling induced by IL-10 on a given cell. By differentially activating STAT1 and STAT3 in different immune cell populations, the engineered IL-10 variants described herein retain the ability to suppress monocyte and macrophage activation, without stimulating T cell functions, thereby uncoupling the immunosuppressive and immunostimulatory functions of IL-10.

The structural basis for receptor engagement by IL-10 provides important insights into the mechanisms underlying IL-10 signaling plasticity and functional pleiotropy. Most notably, the structure shows in molecular detail how IL-10 interacts with its shared, low-affinity receptor subunit IL-10RP, enabling the structure-guided design of mutations that perturb this interface. Analysis of these engineered II,-10 agonists revealed that (i) the plasticity of IL-10 signaling varies between cell types, (ii) these differences can be exploited by modulating the affinity of IL-10 for IL-10Rβ, resulting in altered cell type specificity, and (iii) these cell type biased agonists can uncouple important aspects of IL-10 function. In one aspect of the disclosure, differential IL-10Rβ expression across immune cell populations may represent a natural mechanism to provide functional specificity to otherwise pleiotropic IL-10 mediated responses, a feature that can be exploited using engineered agonists as shown here. The ability of the partial agonist 10-DE to suppress inflammatory macrophage activation without stimulating CD8+ T cells indicates significant implications for targeting the IL-10 receptor in the treatment of relevant health conditions, such as autoimmune and inflammatory diseases.

### Compositions of the Disclosure

As described in greater detail below, one aspect of the present disclosure relates to novel IL-10 polypeptide variants engineered to modulate STAT3 signaling downstream of the IL-10 receptor. Also provided are (i) recombinant nucleic acids encoding such IL-10 polypeptide variants, (ii) recombinant cells that have been engineered to express an IL-10 polypeptide variant as disclosed herein.

### A. Recombinant IL-10 Polypeptides

As outlined above, some embodiments of the disclosure relate to a new class of IL-10 polypeptide variants engineered to modulate STAT3 signaling downstream of the IL-10 receptor, e.g., capable of displaying strong monocyte-biased signaling effects in human PBMCs, by potently suppressing monocyte and macrophage activation/cytokine production, without stimulating cytotoxic CD8+ T cell functions. These IL-10 variants therefore overcome IL-10 pleiotropy and have improved therapeutic potential. In some other embodiments, the IL-10 polypeptide variants of the disclosure confer a cell-type biased STAT3 signaling downstream of the IL-10 receptor.

In one aspect, some embodiments of the disclosure relate to recombinant polypeptides that include: (a) an amino acid sequence having at least 70%, 75%, 80%, 85%, 90%, 91%, 92%, 93%, 94%, 95%, 96%, 97%, 98%, or 99% sequence identity to an interleukin-10 (IL-10) polypeptide having the amino acid sequence of SEQ ID NO: 1 or SEQ ID NO: 16, and further including (b) one or more amino acid substitutions in the sequence of SEQ ID NO: 1 or SEQ ID NO: 16.

Non-limiting exemplary embodiments of the recombinant polypeptides disclosed herein can include one or more of the following features. In some embodiments, the recombinant polypeptides include an amino acid sequence having at least 75%, at least 80%, at least 85%, at least 90%, at least 95%, at least 96%, at least 97%, at least 98%, or at least 99% to the sequence of SEQ ID NO: 1 or SEQ ID NO: 16. In some embodiments, the recombinant polypeptides include an amino acid sequence having 100% sequence identify to the sequence of SEQ ID NO: 1 or SEQ ID NO: 16.

In some embodiments, the recombinant polypeptides include an amino acid sequence having at least 75%, at least 80%, at least 85%, at least 90%, at least 95%, at least 96%, at least 97%, at least 98%, or at least 99% sequence identify to the sequence of SEQ ID NO: 1 or SEQ ID NO: 16, and further include one or more amino acid substitutions at a position corresponding to an amino acid residue selected from the group consisting of X25, X14, X18, X21, X22, X24, X28, X32, X74, X90, X92, X93, X96, X100, and X104 of SEQ ID NO: 1 or SEQ ID NO: 16. In some embodiments, the recombinant polypeptides include an amino acid sequence having at least 75%, at least 80%, at least 85%, at least 90%, at least 95%, at least 96%, at least 97%, at least 98%, or at least 99% sequence identify to the sequence of SEQ ID NO: 1 or SEQ ID NO: 16, and further include one or more amino acid substitutions at a position corresponding to an amino acid residue selected from the group consisting of X25, X14, X18, X24, X28, X74, X90, X92, X96, X100 and X104 of SEQ ID NO: 1 or SEQ ID NO: 16. In some embodiments, the recombinant polypeptides include (a) an amino acid sequence having at least 75%, at least 80%, at least 85%, at least 90%, at least 95%, at least 96%, at least 97%, at least 98%, or at least 99% sequence identity to the amino acid sequence of SEQ ID NO: 1 or SEQ ID NO: 16; and (b) one or more amino acid substitution at a position corresponding to an amino acid residue selected from the group consisting of X25 and X96 of SEQ ID NO: 1 or SEQ ID NO: 16. In some embodiments, the recombinant polypeptides include (a) an amino acid sequence having at least 75%, at least 80%, at least 85%, at least 90%, at least 95%, at least 96%, at least 97%, at least 98%, or at least 99% sequence identity to the amino acid sequence of SEQ ID NO: 1 or SEQ ID NO: 16; and (b) an amino acid substitution at position X25 of SEQ ID NO: 1 or SEQ ID NO: 16. In some embodiments, the recombinant polypeptides include (a) an amino acid sequence having at least 75%, at least 80%, at least 85%, at least 90%, at least 95%, at least 96%, at least 97%, at least 98%, or at least 99% sequence identity to the amino acid sequence of SEQ ID NO: 1 or SEQ ID NO: 16; and (b) an amino acid substitution at position X96 of SEQ ID NO: 1 or SEQ ID NO: 16. In some embodiments, the amino acid sequence of the recombinant polypeptides further include 1, 2, 3, or 4 amino acid substitutions at a position corresponding to an amino acid residue selected from the group consisting of X21, X22, X32, and X93 of SEQ ID NO: 1 or SEQ ID NO: 16. Exemplary IL-10 polypeptide variants according to this aspect can include substitutions of 1, 2, 3, 4, 5, 6, 7, 8, 9, 10 or more amino acids in the sequence of SEQ ID NO: 1 or SEQ ID NO: 16. In some embodiments, the amino acid sequence of the recombinant polypeptides further include at least 1, at least 2, at least 3, at least 4, at least 5, at least 6, or at least 7 amino acid substitutions at a position corresponding to an amino acid residue selected from the group consisting of X25, X14, X18, X24, X28, X74, X90, X92, X96, X100 and X104 of SEQ ID NO: 1 or SEQ ID NO: 16.

In some embodiments, the amino acid sequence of the recombinant polypeptides further include one or more additional amino acid substitutions at a position corresponding to an amino acid residue selected from the group consisting of X21, X22, X32, and X93 of SEQ ID NO: 1 or SEQ ID NO: 16. In some embodiments, the amino acid sequence of the recombinant polypeptides further include one additional amino acid substitution at a position corresponding to an amino acid residue selected from the group consisting of X21 of SEQ ID NO: 1 or SEQ ID NO: 16. In some embodiments, the amino acid sequence of the recombinant polypeptides further include one additional amino acid substitution at a position corresponding to an amino acid residue selected from the group consisting of X22 of SEQ ID NO: 1 or SEQ ID NO: 16. In some embodiments, the amino acid sequence of the recombinant polypeptides further include one additional amino acid substitution at a position corresponding to an amino acid residue selected from the group consisting of X32 of SEQ ID NO: 1. In some embodiments, the amino acid sequence of the recombinant polypeptides further include one additional amino acid substitution at a position corresponding to an amino acid residue selected from the group consisting of X93 of SEQ ID NO: 1 or SEQ ID NO: 16.

In some embodiments, the amino acid substitution(s) is at a position corresponding to an amino acid residue selected from the group consisting of X25, X14, X18, X21, X22, X24, X28, X32, X74, X90, X92, X93, X96, X100 and X104 of SEQ ID NO: 1 or SEQ ID NO: 16. In some embodiments, the polypeptides of the disclosure further include a combination of amino acid substitutions at positions corresponding to amino acid residues X21, X22, X32, and X93 of SEQ ID NO: 1 or SEQ ID NO: 16.

In accordance with this disclosure, any such substitutions in an II,-10 polypeptide result in an IL-10 variant that has an altered binding affinity for IL-10Rβ and/or IL-10Ra, relative to the binding affinity of the parent IL-10 polypeptide lacking such substitutions. For example, the IL-10 polypeptide variants disclosed herein can have increased affinity or decreased affinity for IL-10Rα and/or IL-10Rβ or can have an affinity for these receptors identical or similar to that of the corresponding wild-type IL-10. In some embodiments, the IL-10 polypeptide variants disclosed herein can include conservative modifications and substitutions at other positions of IL-10 (e.g., those that have a minimal effect on the secondary or tertiary structure of the IL-10 variants). Such conservative substitutions include those described by Dayhoff in The Atlas of Protein Sequence and Structure 5 (1978), and by Argos in EMBO J, 8:779-785 (1989). For example, amino acids belonging to one of the following groups represent conservative changes: Group I: Ala, Pro, Gly, Gln, Asn, Ser, Thr; Group II: Cys, Ser, Tyr, Thr; Group III: Val, Ile, Leu, Met, Ala, Phe; Group IV: Lys, Arg, His; Group V: Phe, Tyr, Trp, His; and Group VI: Asp, Glu.

In some embodiments, the amino acid substitution(s) in the amino acid sequence of the recombinant IL-10 polypeptides disclosed herein is independently selected from the group consisting of an alanine substitution, an arginine substitution, an aspartic acid substitution, a histidine substitution, a glutamic acid substitution, a lysine substitution, a serine substitution, a tryptophan substitution, and combinations of any thereof. Non-limiting examples of the amino acid substitutions in the recombinant IL-10 polypeptides disclosed herein are provided in Table 1 below.

TABLE 1

Exemplary amino acid substitutions in the recombinant IL-10 polypeptides of the disclosure

| Position of SEQ ID NO: 1 | Original amino acid | Exemplary substitute amino acid |
|---|---|---|
| 14 | H | A, D, E, I, K, L, M, N, Q, R, S, T, Y, V |
| 18 | N | Y, F, A, D, E, L, V, S, T, I, V, M, H |
| 21 | N | A, R, Q, H, K, S, V, I, L, M, T |
| 22 | M | A V, I, L, N, Q |
| 24 | R | E, D, N, Q, A, S, T |
| 25 | D | A, N, H, I, K, L, V |
| 28 | D | A, E, L, V, S, T, I, V, M, H, K, R |
| 32 | R | A, D, E, L, V, S, T, I, V, M, H |
| 74 | E | A, D, L, V, S, T, I, V, M, H, K, R |
| 90 | H | A, D, E, I, K, L, M, N, Q, R, S, T, Y, V |
| 92 | N | D, Q, E, H, K, S, V, I, L, M, T, A |
| 93 | S | E, A, R, N, D, Q, E, I, L, K, M, V |
| 96 | E | A, N, D, Q, H, K, S |
| 100 | T | D, V, E, A, R, N, Q, E, I, L, K, M, S |
| 104 | R | A, W, Y, F, H, D, E, N, Q, S, T, I, L, V, M |

Accordingly, in some embodiments, a II,-10 partial agonist polypeptide as describe herein can comprise:

SPGQGTQSENSCT(H/A/D/E/I/K/L/M/N/Q/R/S/T/Y/V)FPG(N/F/A/D/E/L/V/S/T/I/V/M/H) LP(N/A/R/Q/H/KSVIL/M/T)(M/A/V/I/L/N/Q)L(R/E/D/N/Q/A/S/T)(D/A/N/H/I/K/L/V)LR( D/A/E/L/V/S/T/I/V/M/H/K/R)AFS(R/A/D/E/L/V/S/T/I/V/M/H)VKTFFQMKDQLDNLLLK ESLLEDFKGYLGCQALSEMIQFYL(E/A/D/L/V/S/T/I/V/M/H/K/R)EVMPQAENQDPDIK A(H/A/D/E/I/K/L/M/N/Q/R/S/T/Y/V)V(N/Q/E/H/K/S/V/I/L/M/T/A)(S/E/A/R/N/D/Q/E/I/L/ K/M/V)LG(E/A/N/D/Q/H/K/S)NLK(T/V/E/A/R/N/Q/E/I/L/K/M/S)LRL(R/A/W/Y/F/H/D/E/ N/Q/S/T/I/L/V/M)LRRCHRFLPCENKSKAVEQVKNAFNKLQEKGIYKAMSEFDIFINYI EAYMTMKIRN (SEQ ID NO: 32), wherein at least one (e.g., 1, 2, 3, 4, 5, 6, or more) of the variable positions are not the wildtype amino acid that occurs in the corresponding position in SEQ ID NO: 1 and wherein alternatives at positions are shown in parentheses.

In some embodiments, the recombinant polypeptides include an amino acid sequence having 100% sequence identify to the sequence of SEQ ID NO: 1 or SEQ ID NO: 16, and further include one or more amino acid substitutions at a position corresponding to an amino acid residue selected from the group consisting of D25, H14, N18, N21, M22, R24, D28, R32, E74, H90, N92, S93, E96, T100 and R104 of SEQ ID NO: 1 or SEQ ID NO: 16. In some embodiments, the amino acid substitution(s) is at a position corresponding to an amino acid residue selected from the group consisting of D25, H14, N18, R24, D28, E74, H90, N92, E96, T100 and R104 of SEQ ID NO: 1 or SEQ ID NO: 16. In some embodiments, the amino acid substitution(s) is at a position corresponding to an amino acid residue selected from the group consisting of D25 and E96 of SEQ ID NO: 1 or SEQ ID NO: 16. In some embodiments, the amino acid substitution(s) is at position D25 of SEQ ID NO: 1 or SEQ ID NO: 16. In some embodiments, the amino acid substitution(s) at position D25 of SEQ ID NO: 1 or SEQ ID NO: 16 is a D25A substitution. In some embodiments, the amino acid substitution(s) at position D25 of SEQ ID NO: 1 or SEQ ID NO: 16 is a D25K substitution. In some embodiments, the amino acid substitution(s) is at position E96 of SEQ ID NO: 1 or SEQ ID NO: 16. In some embodiments, the amino acid substitution(s) at position E96 of SEQ ID NO: 1 or SEQ ID NO: 16 is an E96A substitution. In some embodiments, the amino acid substitution(s) at position E96 of SEQ ID NO: 1 or SEQ ID NO: 16 is an E96K substitution. In some embodiments, the amino acid substitution(s) is at a position corresponding to an amino acid residue selected from the group consisting of N21, M22, R32, and S93 of SEQ ID NO: 1 or SEQ ID NO: 16.

In some embodiments, the polypeptides of the disclosure include an amino acid sequence having at least 70%, 75%, 80%, 85%, 90%, 91%, 92%, 93%, 94%, 95%, 96%, 97%, 98%, or 99% sequence identity to SEQ ID NO: 1 or SEQ ID NO: 16, and further include the amino acid substitutions corresponding to the following amino acid substitutions: (a) N18Y/N92Q/T100D/R104W or R104A; (b) N18Y/N21H/N92Q/E96D/T100V/R104W or R104A; (c) N18Y/N21H/E96H/T100V/R104W or R104A; (d) N18Y/D25A or D25K/N92Q/T100D/R104W or R104A; (e) N18Y/D12A or D25K/N92Q/T100D/R104W or R104A; and (f) N18Y/D25A or D25K/N92Q/E96A/T100D/R104W or R104A.

In some embodiments, the polypeptides of the disclosure include an amino acid sequence having at least 70%, 75%, 80%, 85%, 90%, 91%, 92%, 93%, 94%, 95%, 96%, 97%, 98%, or 99% sequence identity to SEQ ID NO: 1 or SEQ ID NO: 16, and further include the amino acid substitutions corresponding to the following amino acid substitutions: (a) N18Y/N92Q/T100D/R104W; (b) N18Y/N21H/N92Q/E96D/T100V/R104W; (c) N18Y/N21H/E96H/T100V/R104W; (d) N18Y/D25A/N92Q/T100D/R104W; (e) N18Y/D25K/N92Q/T100D/R104W; and (f) N18Y/D25A/N92Q/E96A/T100D/R104W. In some embodiments, the polypeptides of the disclosure include an amino acid sequence having at least 70%, 75%, 80%, 85%, 90%, 91%, 92%, 93%, 94%, 95%, 96%, 97%, 98%, or 99% sequence identity to SEQ ID NO: 1 or SEQ ID NO: 16, and further include the amino acid substitutions corresponding to the following amino acid substitutions: (a) N18Y/N92Q/T100D/R104A; (b) N18Y/N21H/N92Q/E96D/T100V/R104A; (c) N18Y/N21H/E96H/T100V/R104A; (d) N18Y/D25A/N92Q/T100D/R104A; (e) N18Y/D25K/N92Q/T100D/R104A; and (f) N18Y/D25A/N92Q/E96A/T100D/R104A.

In some embodiments, the polypeptides of the disclosure include an amino acid sequence having at least 70% , 75%, 80%, 85%, 90%, 91%, 92%, 93%, 94%, 95%, 96%, 97%, 98%, or 99% sequence identity to SEQ ID NO: 1 or SEQ ID NO: 16, and further include the amino acid substitutions corresponding to the following amino acid substitutions: (a) N18Y/N92Q/T100D/R104W; and (b) N21A. In some embodiments, the polypeptides of the disclosure include an amino acid sequence having at least 70% , 75%, 80%, 85%, 90%, 91%, 92%, 93%, 94%, 95%, 96%, 97%, 98%, or 99% sequence identity to SEQ ID NO: 1 or SEQ ID NO: 16, and further include the amino acid substitutions corresponding to the following amino acid substitutions: (a) N18Y/N92Q/T100D/R104A; and (b) N21A. In some embodiments, the polypeptides of the disclosure include an amino acid sequence having at least 70% , 75%, 80%, 85%, 90%, 91%, 92%, 93%, 94%, 95%, 96%, 97%, 98%, or 99% sequence identity to SEQ ID NO: 1 or SEQ ID NO: 16, and further include the amino acid substitutions corresponding to the following amino acid substitutions: (a) N18Y/N92Q/T100D/R104W; and (b) one or more mutations selected from the group consisting of D25A, D25K, and D25A/E96A. In some embodiments, the polypeptides of the disclosure include an amino acid sequence having at least 70% , 75%, 80%, 85%, 90%, 91%, 92%, 93%, 94%, 95%, 96%, 97%, 98%, or 99% sequence identity to SEQ ID NO: 1 or SEQ ID NO: 16, and further include the amino acid substitutions corresponding to the following amino acid substitutions: (a) N18Y/N92Q/T100D/R104A; and (b) one or more mutations selected from the group consisting of D25A, D25K, and D25A/E96A.

In some embodiments, the polypeptides of the disclosure include an amino acid sequence having at least 75%, at least 80%, at least 85%, at least 90%, at least 95%, at least 96%, at least 97%, or at least 98%, at least 99% sequence identity to SEQ ID NO: 1 or SEQ ID NO: 16, and further include the amino acid substitutions corresponding to the following amino acid substitutions: (a) N18Y/N92Q/T100D/R104W; (b) N18Y/N21H/N92Q/E96D/T100V/R104W; (c) N18Y/N21H/E96H/T100V/R104W; (d) N18Y/D25A/N92Q/T100D/R104W; (e) N18Y/D25K/N92Q/T100D/R104W; and (f) N18Y/D25A/N92Q/E96A/T100D/R104W. In some embodiments, the polypeptides of the disclosure include an amino acid sequence having at least 75%, at least 80%, at least 85%, at least 90%, at least 95%, at least 96%, at least 97%, or at least 98%, at least 99% sequence identity to SEQ ID NO: 1 or SEQ ID NO: 16, and further include the amino acid substitutions corresponding to the following amino acid substitutions: (a) N18Y/N92Q/T100D/R104A; (b) N18Y/N21H/N92Q/E96D/T100V/R104A; (c) N18Y/N21H/E96H/T100V/R104A; (d) N18Y/D25A/N92Q/T100D/R104A; (e) N18Y/D25K/N92Q/T100D/R104A; and (f) N18Y/D25A/N92Q/E96A/T100D/R104A. In some embodiments, the polypeptides of the disclosure include an amino acid sequence having 100% sequence identity to SEQ ID NO: 1 or SEQ ID NO: 16, and further include the amino acid substitutions corresponding to the following amino acid substitutions: (a) N18Y/N92Q/T100D/R104W; (b) N18Y/N21H/N92Q/E96D/T100V/R104W; (c) N18Y/N21H/E96H/T100V/R104W; (d) N18Y/D25A/N92Q/T100D/R104W; (e) N18Y/D25K/N92Q/T100D/R104W; and (f) N18Y/D25A/N92Q/E96A/T100D/R104W. In some embodiments, the polypeptides of the disclosure include an amino acid sequence having 100% sequence identity to SEQ ID NO: 1 or SEQ ID NO: 16, and further include the amino acid substitutions corresponding to the following amino acid substitutions: (a) N18Y/N21H/N92Q/E96D/T100V/R104W; and (b) N21A. In some embodiments, the polypeptides of the disclosure include an amino acid sequence having 100% sequence identity to SEQ ID NO: 1 or SEQ ID NO: 16, and further include the amino acid substitutions corresponding to the following amino acid substitutions: (a) N18Y/N21H/N92Q/E96D/T100V/R104W; and (b) one or more mutations selected from the group consisting of D25A, D25K, and D25A/E96A. In some embodiments, the polypeptides of the disclosure include an amino acid sequence having 100% sequence identity to SEQ ID NO: 1 or SEQ ID NO: 16, and further include the amino acid substitutions corresponding to the following amino acid substitutions: (a) N18Y/N92Q/T100D/R104A; (b) N18Y/N21H/N92Q/E96D/T100V/R104A; (c) N18Y/N21H/E96H/T100V/R104A; (d) N18Y/D25A/N92Q/T100D/R104A; (e) N18Y/D25K/N92Q/T100D/R104A; and (f) N18Y/D25A/N92Q/E96A/T100D/R104A. In some embodiments, the polypeptides of the disclosure include an amino acid sequence having 100% sequence identity to SEQ ID NO: 1 or SEQ ID NO: 16, and further include the amino acid substitutions corresponding to the following amino acid substitutions: (a) N18Y/N21H/N92Q/E96D/T100V/R104A; and (b) N21A. In some embodiments, the polypeptides of the disclosure include an amino acid sequence having 100% sequence identity to SEQ ID NO: 1 or SEQ ID NO: 16, and further include the amino acid substitutions corresponding to the following amino acid substitutions: (a) N18Y/N21H/N92Q/E96D/T100V/R104A; and (b) one or more mutations selected from the group consisting of D25A, D25K, and D25A/E96A.

In some embodiments, the polypeptides of the disclosure include an amino acid sequence having at least 70%, 75%, 80%, 85%, 90%, 91%, 92%, 93%, 94%, 95%, 96%, 97%, 98%, or 99% sequence identity to SEQ ID NO: 1 or SEQ ID NO: 16, and further include the amino acid substitutions corresponding to the following amino acid substitutions: (a) D25A; (b) D25K; (c) E96A; (d) E96K; (e) D25A/E96A; (f) N21A/R104A; (g) N21A/D25A; (h) N21A/D25A/E96A; and (i) N21A/M22A/D25A. In some embodiments, the polypeptides of the disclosure include an amino acid sequence having at least 75%, at least 80%, at least 85%, at least 90%, at least 95%, at least 96%, at least 97%, or at least 98%, at least 99% sequence identity to SEQ ID NO: 1 or SEQ ID NO: 16, and further include the amino acid substitutions corresponding to the following amino acid substitutions: (a) D25A; (b) D25K; (c) E96A; (d) E96K; (e) D25A/E96A; (f) N21A/R104A; (g) N21A/D25A; (h) N21A/D25A/E96A; and (i) N21A/M22A/D25A. In some embodiments, the polypeptides of the disclosure include an amino acid sequence having 100% sequence identity to SEQ ID NO: 1 or SEQ ID NO: 16, and further include the amino acid substitutions corresponding to the following amino acid substitutions: (a) D25A; (b) D25K; (c) E96A; (d) E96K; (e) D25A/E96A; (f) N21A/R104A; (g) N21A/D25A; (h) N21A/D25A/E96A; and (i) N21A/M22A/D25A.

In some embodiments, the recombinant polypeptides include an amino acid sequence having at least 70%, 75%, 80%, 85%, 90%, 91%, 92%, 93%, 94%, 95%, 96%, 97%, 98%, or 99% sequence identity to the sequence of SEQ ID NO: 1 or SEQ ID NO: 16, and further include an amino acid substitution corresponding an amino acid residue selected from the group consisting of D25, H14, N18, N21, M22, R24, D28, R32, E74, H90, N92, S93, E96, T100 and R104 of SEQ ID NO: 1 or SEQ ID NO: 16. In some embodiments, the recombinant polypeptides include an amino acid sequence having at least 75%, at least 80%, at least 85%, at least 90%, at least 95%, at least 96%, at least 97%, at least 98%, at least 99%, or at least 100 % sequence identity to the sequence of SEQ ID NO: 1 or SEQ ID NO: 16, and further include an amino acid substitution corresponding an amino acid residue selected from the group consisting of D25, H14, N18, N21, M22, R24, D28, R32, E74, H90, N92, S93, E96, T100 and R104 of SEQ ID NO: 1 or SEQ ID NO: 16. In some embodiments, the amino acid sequence of the recombinant polypeptides further include at least 1, at least 2, at least 3, at least 4, at least 5, at least 6, or at least 7 amino acid substitutions at a position corresponding to an amino acid residue selected from the group consisting of D25, H14, N18, N21, M22, R24, D28, R32, E74, H90, N92, S93, E96, T100 and R104 of SEQ ID NO: 1 or SEQ ID NO: 16.

In some embodiments, the recombinant polypeptides include an amino acid sequence having at least 70%, 75%, 80%, 85%, 90%, 91%, 92%, 93%, 94%, 95%, 96%, 97%, 98%, or 99% sequence identity to the sequence of SEQ ID NO: 1 or SEQ ID NO: 16, and further include an amino acid substitution corresponding an amino acid residue selected from the group consisting of D25, H14, N18, N21, M22, R24, D28, R32, E74, H90, N92, S93, E96, T100 and R104 of SEQ ID NO: 1 or SEQ ID NO: 16. In some embodiments, the recombinant polypeptides include an amino acid sequence having at least 75%, at least 80%, at least 85%, at least 90%, at least 95%, at least 96%, at least 97%, at least 98%, at least 99%, or at least 100 % sequence identity to the sequence of SEQ ID NO: 1 or SEQ ID NO: 16, and further include an amino acid substitution corresponding an amino acid residue selected from the group consisting of D25, H14, N18, N21, M22, R24, D28, R32, E74, H90, N92, S93, E96, T100 and R104 of SEQ ID NO: 1 or SEQ ID NO: 16. In some embodiments, the amino acid sequence of the recombinant polypeptides further include at least 1, at least 2, at least 3, at least 4, at least 5, at least 6, or at least 7 amino acid substitutions at a position corresponding to an amino acid residue selected from the group consisting of D25, H14, N18, N21, M22, R24, D28, R32, E74, H90, N92, S93, E96, T100 and R104 of SEQ ID NO: 1 or SEQ ID NO: 16. In some embodiments, the amino acid sequence further includes at least one additional amino acid substitution at a position corresponding to an amino acid residue selected from the group consisting of N21, M22, R32, and S93 of SEQ ID NO: 1 or SEQ ID NO: 16.

In some embodiments, the recombinant polypeptides of the disclosure can include an amino acid sequence that is set forth herein except for one or more (e.g., 1, 2, 3, 4, 5, 6, 7, 8, 9 or 10) mutations such as, for example, missense mutations (e.g., conservative substitutions), nonsense mutations, deletions, or insertions. In some embodiments disclosed herein, the recombinant polypeptides of the disclosure include an amino acid sequence having at least 90%, 95%, 96%, 97, 98%, 99% sequence identity to an amino acid sequence selected from the group consisting of SEQ ID NOS: 2-15. In some embodiments, the recombinant polypeptides of the disclosure include an amino acid sequence having at least 90%, 95%, 96%, 97, 98%, 99%, or 100% sequence identity to the amino acid sequence of SEQ ID NO: 2. In some embodiments, the recombinant polypeptides include an amino acid sequence having at least 90%, 95%, 96%, 97, 98%, 99%, or 100% sequence identity to the amino acid sequence of SEQ ID NO: 3. In some embodiments, the recombinant polypeptides include an amino acid sequence having 90%, 95%, 96%, 97, 98%, 99%, or 100% sequence identity to the amino acid sequence of SEQ ID NO: 4. In some embodiments, the recombinant polypeptides include an amino acid sequence having 90%, 95%, 96%, 97, 98%, 99%, or 100% sequence identity to the amino acid sequence of SEQ ID NO: 5. In some embodiments, the recombinant polypeptides include an amino acid sequence having 90%, 95%, 96%, 97, 98%, 99%, or 100% sequence identity to the amino acid sequence of SEQ ID NO: 6. In some embodiments, the recombinant polypeptides include an amino acid sequence having 90%, 95%, 96%, 97, 98%, 99%, or 100% sequence identity to the amino acid sequence of SEQ ID NO: 7.

In some embodiments, the recombinant polypeptides of the disclosure include an amino acid sequence having at least 90%, 95%, 96%, 97, 98%, 99%, or 100% sequence identity to the amino acid sequence of SEQ ID NO: 8. In some embodiments, the recombinant polypeptides include an amino acid sequence having at least 90%, 95%, 96%, 97, 98%, 99%, or 100% sequence identity to the amino acid sequence of SEQ ID NO: 9. In some embodiments, the recombinant polypeptides include an amino acid sequence having 90%, 95%, 96%, 97, 98%, 99%, or 100% sequence identity to the amino acid sequence of SEQ ID NO: 10. In some embodiments, the recombinant polypeptides include an amino acid sequence having 90%, 95%, 96%, 97, 98%, 99%, or 100% sequence identity to the amino acid sequence of SEQ ID NO: 11. In some embodiments, the recombinant polypeptides include an amino acid sequence having 90%, 95%, 96%, 97, 98%, 99%, or 100% sequence identity to the amino acid sequence of SEQ ID NO: 12. In some embodiments, the recombinant polypeptides include an amino acid sequence having 90%, 95%, 96%, 97, 98%, 99%, or 100% sequence identity to the amino acid sequence of SEQ ID NO: 13. In some embodiments, the recombinant polypeptides include an amino acid sequence having 90%, 95%, 96%, 97, 98%, 99%, or 100% sequence identity to the amino acid sequence of SEQ ID NO: 14. In some embodiments, the recombinant polypeptides include an amino acid sequence having 90%, 95%, 96%, 97, 98%, 99%, or 100% sequence identity to the amino acid sequence of SEQ ID NO: 15. In some embodiments, the amino acid substitution(s) in the sequence of the recombinant II,-10 polypeptide disclosed herein results in a modulation of binding affinity of the recombinant IL-10 polypeptide for IL-10RJ3. The term “modulating”, in relation to the binding activity of an IL-10 polypeptide refers to a change in the binding affinity of the polypeptide for IL-1 ORB. Modulation includes both increase (e.g., induce, stimulate) and decrease (e.g., reduce, inhibit), or otherwise affecting the binding affinity of the polypeptide. In some embodiments, the amino acid substitution(s) in the sequence of the recombinant IL-10 polypeptide disclosed herein reduces IL-10RP-binding affinity of the recombinant IL-10 polypeptide compared to a reference IL-10 polypeptide lacking the amino acid substitution(s). In some embodiments, the amino acid substitution(s) increases IL-10RJ3-binding affinity of the recombinant IL-10 polypeptide compared to a reference IL-10 polypeptide lacking the amino acid substitution(s).

The binding activity of recombinant polypeptides of the disclosure, including the IL-10 polypeptide variants described herein, can be assayed by any suitable method known in the art. For example, the binding activity of an IL-10 polypeptide variant disclosed herein and its receptors (e.g., IL-10Rα and/or IL-10RJ3) can be determined by Scatchard analysis (Munsen et al. Analyt. Biochem. 107:220-239, 1980). Specific binding may also be assessed using techniques known in the art including but not limited to competition ELISA, Biacore® assays and/or KinExA® assays. A polypeptide that preferentially binds or specifically binds to a target protein is a concept well understood in the art, and methods to determine such specific or preferential binding are also known in the art.

A variety of assay formats may be used to select a recombinant IL-10 polypeptide that specifically binds a ligand of interest (e.g., IL-10Rα and/or IL-10RB). For example, solid-phase ELISA immunoassay, immunoprecipitation, Biacore™ (GE Healthcare, Piscataway, NJ), KinExA, fluorescence-activated cell sorting (FACS), Octet™ (ForteBio, Inc., Menlo Park, CA) and Western blot analysis are among many assays that may be used to identify a polypeptide that specifically reacts with a receptor or a ligand binding portion thereof, that specifically binds with a cognate ligand or binding partner. Generally, a specific or selective binding reaction will be at least twice the background signal or noise, more typically more than 10 times background, more than 20 times background, even more typically, more than 50 times background, more than 75 times background, more than 100 times background, yet more typically, more than 500 times background, even more typically, more than 1000 times background, and even more typically, more than 10,000 times background.

One of ordinary skill in the art will appreciate that binding affinity can also be used as a measure of the strength of a non-covalent interaction between two molecules, e.g., an IL-10 polypeptide and an IL-10Rβ receptor. In some instance, binding affinity is used to describe monovalent interactions (intrinsic activity). Binding affinity between two molecules may be quantified by determination of the dissociation constant (K_{D}). In turn, K_{D} can be determined by measurement of the kinetics of complex formation and dissociation using, e.g., the surface plasmon resonance (SPR) method (Biacore). The rate constants corresponding to the association and the dissociation of a monovalent complex are referred to as the association rate constants kₐ (or kₒₙ) and dissociation rate constant k_{d} (or k_{off}), respectively. K_{D} is related to kₐ and k_{d} through the equation K_{D} = kd / kₐ. The value of the dissociation constant can be determined directly by well-known methods and can be computed even for complex mixtures by methods such as those set forth in Caceci et al. (Byte 9: 340-362, 1984). For example, the K_{D} may be established using a double-filter nitrocellulose filter binding assay such as that disclosed by Wong & Lohman (1993, Proc. Natl. Acad. Sci. USA 90: 5428- 5432). Other standard assays to evaluate the binding ability of the IL-10 polypeptide variants of the present disclosure towards target receptors are known in the art, including for example, ELISAs, Western blots, RIAs, and flow cytometry analysis, and other assays exemplified in the Examples. The binding kinetics and binding affinity of the IL-10 polypeptide variants also can be assessed by standard assays known in the art, such as Surface Plasmon Resonance (SPR), e.g. by using a Biacore™ system, or KinExA. In some embodiments, the binding affinity of the IL-10 polypeptide variant of the disclosure to IL-10Rα and/or IL-10Rβ is determined by a solid-phase receptor binding assay (Matrosovich MN et al., Methods Mol Biol. 865:71-94, 2012). In some embodiments, the binding affinity of the IL-10 polypeptide variant of the disclosure to IL-10Rα and/or IL-10Rβ is determined by a Surface Plasmon Resonance (SPR) assay.

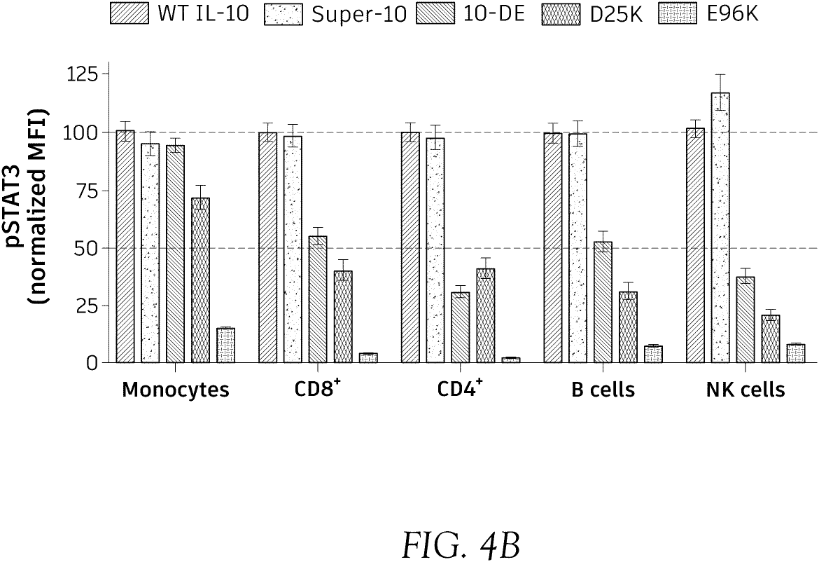

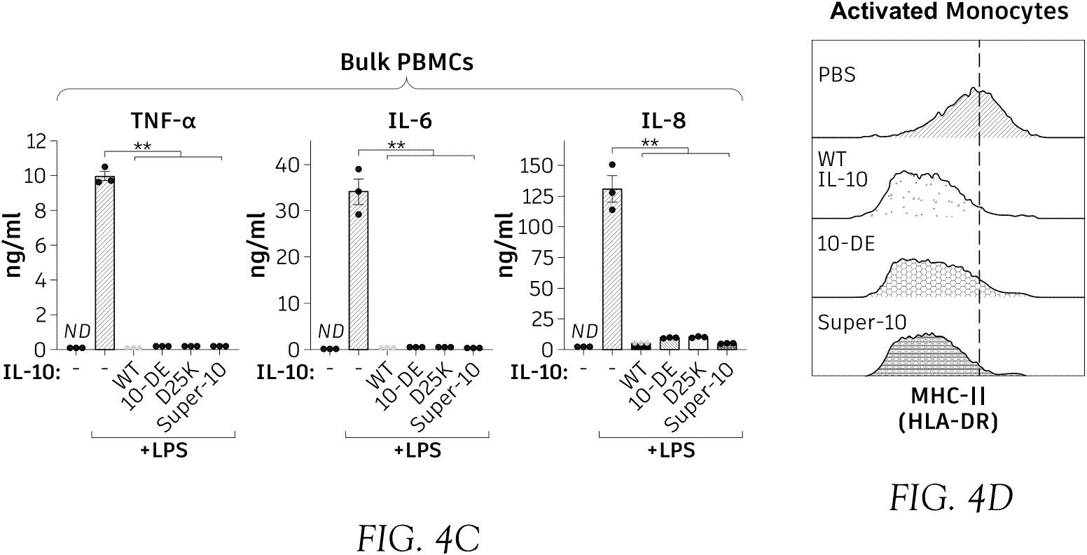

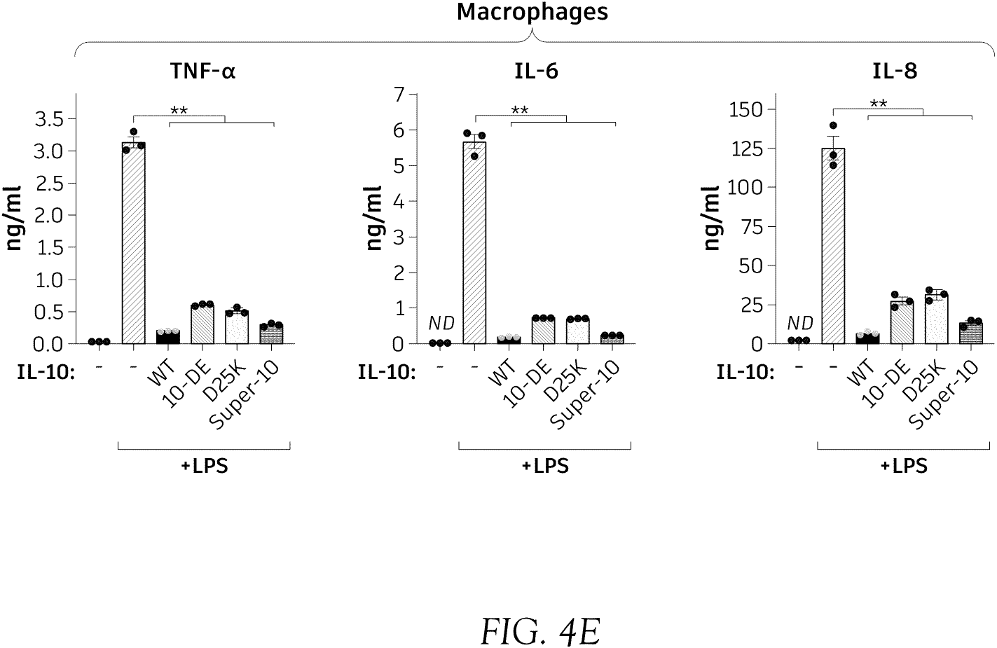

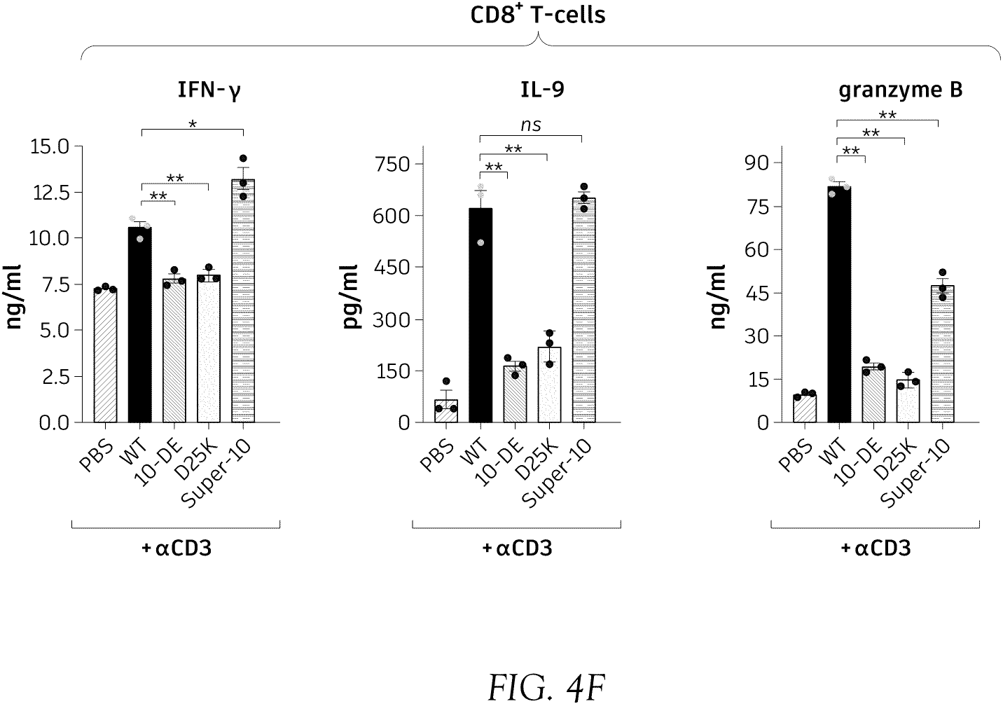

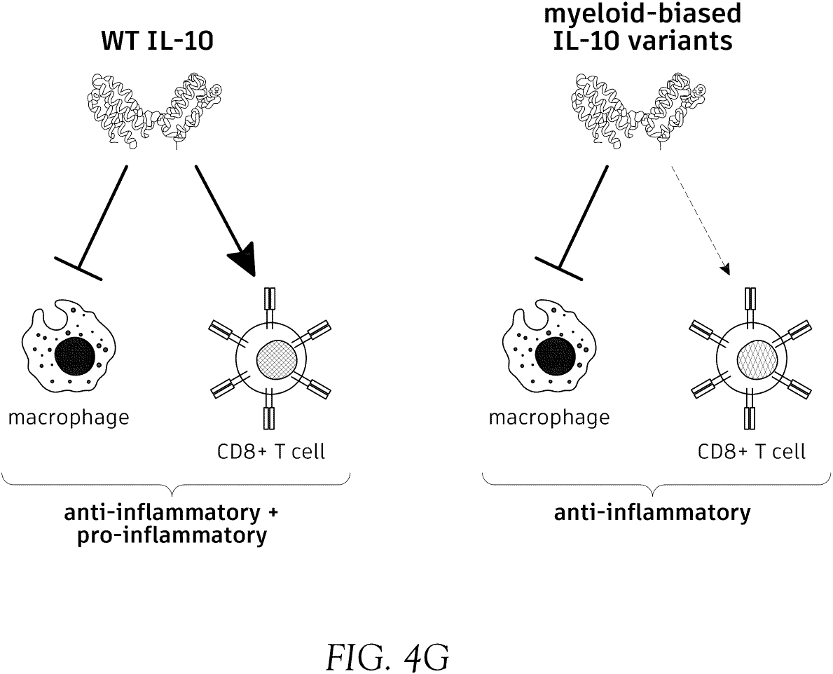

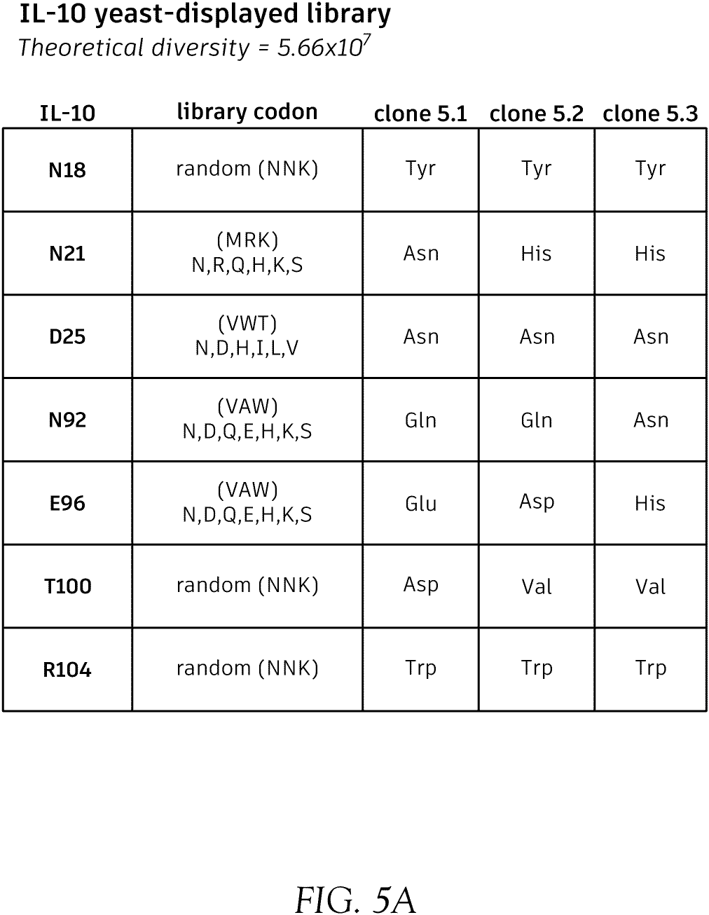

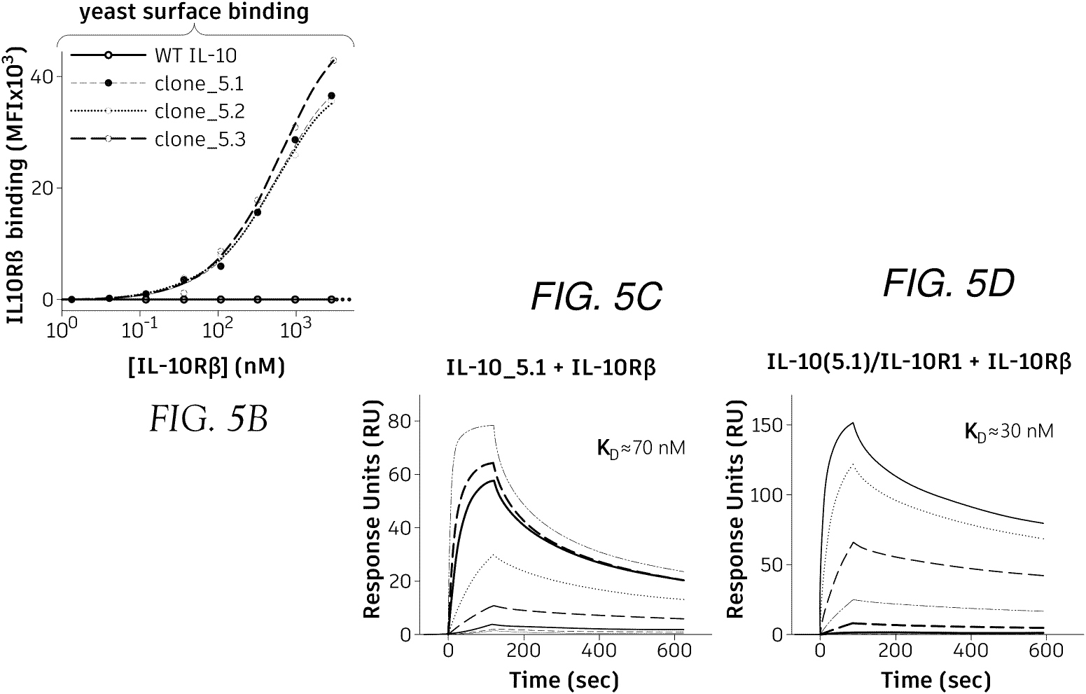

In some embodiments, the amino acid substitutions in the recombinant IL-10 polypeptide variants disclosed herein result in a cell-type biased IL-10 signaling compared to a reference IL-10 polypeptide lacking the amino acid substitutions. As described in further detail below, an exemplary IL-10 polypeptide variant of the disclosure, 10-DE, elicits cell-type biased signaling responses in vivo, by exhibiting monocyte-biased signaling in human PBMCs but suppressing inflammatory cytokine production in bulk PBMCs. The 10-DE variant was also found to strongly downregulate surface MHC class II expression on LPS-stimulated monocytes (see, e.g., Example 6 and FIG. 4 below). Importantly, 10-DE variant also fully abrogated LPS-induced TNF-a, IL-6, and IL-8 production in isolated monocyte-derived macrophages (FIG. 4E), the key target for IL-10 in autoimmune and auto-inflammatory diseases. Accordingly, in some embodiments, the amino acid substitutions in the recombinant IL-10 polypeptide variants disclosed herein result in a cell-type biased IL-10 signaling which involves a reduction of IL-10 signaling in NK cells, B cells, CD4+ T cells, and/or CD8+ T cells while substantially retains IL-10 signaling in monocytes and macrophages.

In some embodiments, the amino acid substitution(s) in the IL-10 polypeptide variants disclosed herein results in a cell-type biased IL-10 signaling as determined by, for example, phosphorylation of STAT3, compared to a reference IL-10 polypeptide lacking the amino acid substitution(s). In some embodiments, the biased IL-10 signaling includes a reduction in a STAT3 phosphorylation in one or more cell types, such as B cells, T cells, and/or NK cells by at least 10%, e.g., by at least 10%, at least 20%, at least 30%, at least 40%, at least 50%, at least 60%, at least 70%, at least 80%, or at least 90% when compared to a reference IL-10 polypeptide lacking the amino acid substitution(s). In some embodiments, the administered composition does not potentiate activity of activated CD8+ T cells. In some embodiments, the administered composition does not potentiate IFN-γ production and/or Granzyme B production by CD8+ T cells.

In some embodiments, the amino acid substitution(s) in the IL-10 polypeptide variants disclosed herein results in a reduction in one or more STAT3-mediated pro-inflammatory functions in one or more cell types. There are no specific limitations to the STAT3-mediated functions that can be suitably assayed. Non-limiting examples of suitable STAT3-mediated pro-inflammatory function include cytokine production, chemokine production, and immune cell recruitment. In some embodiments, the STAT3-mediated pro-inflammatory function is reduced about 20% to about 100% in B cells, T cells, and/or NK cells. In some embodiments, the STAT3 signaling is determined by an assay selected from the group consisting of by a gene expression assay, a phospho-flow signaling assay, and an enzyme-linked immunosorbent assay (ELISA). In some embodiments, the amino acid substitution(s) in the IL-10 polypeptide variants disclosed herein results in a reduction in one or more STAT3-mediated pro-inflammatory functions, as determined by the polypeptides’ capacity to induce expression of a pro-inflammatory gene and/or MHC class II expression. Non-limiting examples of pro-inflammatory genes include IFN-y, granzyme B, granzyme A, perforin, TNF-α, GM-CSF, and MIP1α. In some embodiments, the STAT3-mediated pro-inflammatory function is reduced about 20% to about 100%, for example, about 20% to about 50%, about 30% to about 60%, about 40% to about 70%, about 50% to about 80%, about 40% to about 90%, bout 50% to about 100%, about 40% to about 80%, about 30% to about 70%, about 20% to about 80%, about 20% to about 70%, about 20% to about 60%, about 30% to about 80%, about 30% to about 90%, or about 30% to about 100% compared to a reference IL-10 lacking the amino acid substitution(s).

In some embodiments, the amino acid substitution(s) in the IL-10 polypeptide variants disclosed herein results in a reduction in a STAT3-mediated pro-inflammatory function in a cell type selected from B cells, CD4+ T cells, CD8+ T cells, and NK cells while substantially retains its STAT3-mediated function in monocytes and/or macrophages. In some embodiments, the STAT3 signaling is determined by an assay selected from the group consisting of by a gene expression assay, a phospho-flow signaling assay, and an enzyme-linked immunosorbent assay (ELISA).

In some embodiments, the amino acid substitution(s) in the IL-10 polypeptide variants disclosed herein results in an increased binding affinity for IL-10Rβ compared to binding affinity of the reference IL-10 polypeptide (e.g., wild-type IL-10). As described in greater detail below, increased binding affinity for IL-10Rβ of such IL-10 polypeptide variants (e.g., “super-10” variant) results in stimulation of INFγ expression in CD8+ T cells. Without being bound to any particular theory, such IL-10 variants with increased binding affinity can be useful in methods of treating relevant proliferative diseases, such as cancer.

In some embodiments, the amino acid substitution(s) in the IL-10 polypeptide variants disclosed herein results in a reduced binding affinity for IL-10Rβ compared to binding affinity of the reference IL-10 polypeptide (e.g., wild-type IL-10). As described in greater detail below, reduced binding affinity for IL-10Rβ of such IL-10 polypeptide variants (e.g., partial agonist 10-DE) results in a reduced capability to stimulate STAT3 signaling and/or in a suppression of inflammatory macrophage activation without stimulating CD8+ T cells. Without being bound to any particular theory, such IL-10 variants with reduced binding affinity can be useful in methods of relevant health conditions, such as autoimmune and inflammatory diseases.

### B. Nucleic Acids

In one aspect, provided herein are various nucleic acid molecules including nucleotide sequences encoding the recombinant IL-10 polypeptides the disclosure, including expression cassettes, and expression vectors containing these nucleic acid molecules operably linked to heterologous nucleic acid sequences such as, for example, regulator sequences which allow in vivo expression of the recombinant IL-10 polypeptide in a host cell or ex-vivo cell-free expression system.

The terms “nucleic acid molecule” and “polynucleotide” are used interchangeably herein, and refer to both RNA and DNA molecules, including nucleic acid molecules comprising cDNA, genomic DNA, synthetic DNA, and DNA or RNA molecules containing nucleic acid analogs. A nucleic acid molecule can be double-stranded or single-stranded (e.g., a sense strand or an antisense strand). A nucleic acid molecule may contain unconventional or modified nucleotides. The terms “polynucleotide sequence” and “nucleic acid sequence” as used herein interchangeably refer to the sequence of a polynucleotide molecule. The polynucleotide and polypeptide sequences disclosed herein are shown using standard letter abbreviations for nucleotide bases and amino acids as set forth in 37 CFR §1.82), which incorporates by reference WIPO Standard ST.25 (1998), Appendix 2, Tables 1-6.

Nucleic acid molecules of the present disclosure can be nucleic acid molecules of any length, including nucleic acid molecules that are generally between about 0.5 Kb and about 20 Kb, for example between about 0.5 Kb and about 20 Kb, between about 1 Kb and about 15 Kb, between about 2 Kb and about 10 Kb, or between about 5 Kb and about 25 Kb, for example between about 10 Kb to 15 Kb, between about 15 Kb and about 20 Kb, between about 5 Kb and about 20 Kb, about 5 Kb and about 10 Kb, or about 10 Kb and about 25 Kb.

In some embodiments disclosed herein, the nucleic acid molecules of the disclosure include a nucleotide sequence encoding a polypeptide which includes an amino acid sequence having at least 70%, at least 75%, at least 80%, at least 85%, at least 90%, at least 95%, at least 96%, at least 97, at least 98%, at least 99%, or at least 100% sequence identity to the amino acid sequence of a recombinant polypeptide as disclosed herein. In some embodiments, the nucleic acid molecules of the disclosure include a nucleotide sequence encoding a polypeptide that includes: (a) an amino acid sequence having at least 70%, 80%, 90%, 95%, 99%, or 100% sequence identity to an IL-10 polypeptide having the amino acid sequence of SEQ ID NO: 1 or SEQ ID NO: 16; and further including (b) one or more amino acid substitution at a position corresponding to an amino acid residue selected from the group consisting of X25, X14, X18, X21, X22, X24, X28, X32, X74, X90, X92, X93, X96, X100 and X104 of SEQ ID NO: 1 or SEQ ID NO: 16. In some embodiments, the nucleic acid molecules of the disclosure include a nucleotide sequence encoding a polypeptide that includes: (a) an amino acid sequence having at least 70%, 80%, 90%, 95%, 99%, or 100% sequence identity to an IL-10 polypeptide having the amino acid sequence of SEQ ID NO: 1 or SEQ ID NO: 16; and further including (b) one or more amino acid substitution at a position corresponding to an amino acid residue selected from the group consisting of X25, X14, X18, X24, X28, X74, X90, X92, X96, X100 and X104 of SEQ ID NO: 1 or SEQ ID NO: 16. In some embodiments, the polypeptide further includes at least one additional amino acid substitution at a position corresponding to an amino acid residue selected from the group consisting of X21, X22, X32, and X93 of SEQ ID NO: 1 or SEQ ID NO: 16. In some embodiments, the nucleic acid molecules of the disclosure include a nucleotide sequence encoding a polypeptide that includes an amino acid sequence having at least 70%, 80%, 90%, 95%, 99%, or 100% sequence identity to SEQ ID NO: 1 or SEQ ID NO: 16, and further include an amino acid substitution corresponding an amino acid residue selected from the group consisting of D25, H14, N18, N21, M22, R24, D28, R32, E74, H90, N92, S93, E96, T100 and R104 of SEQ ID NO: 1 or SEQ ID NO: 16. In some embodiments, the nucleic acid molecules of the disclosure include a nucleotide sequence encoding a polypeptide that includes an amino acid sequence having at least 70%, 80%, 90%, 95%, 99%, or 100% sequence identity to SEQ ID NO: 1 or SEQ ID NO: 16, and further include an amino acid substitution corresponding an amino acid residue selected from the group consisting of D25, H14, N18, R24, D28, E74, H90, N92, E96, T100 and R104 of SEQ ID NO: 1 or SEQ ID NO: 16. In some embodiments, the amino acid sequence of the recombinant polypeptides further include one or more additional amino acid substitutions at a position corresponding to an amino acid residue selected from the group consisting of N21, M22, R32, and S93 of SEQ ID NO: 1 or SEQ ID NO: 16.

In some embodiments, the nucleic acid molecules of the disclosure include a nucleotide sequence encoding a polypeptide that includes an amino acid sequence having at least 70%, 75%, 80%, 85%, 90%, 91%, 92%, 93%, 94%, 95%, 96%, 97%, 98%, or 99% sequence identity to SEQ ID NO: 1 or SEQ ID NO: 16, and further include the amino acid substitutions corresponding to the following amino acid substitutions: (a) N18Y/N92Q/T100D/R104W or R104A; (b) N18Y/N21H/N92Q/E96D/T100V/R104W or R104A; (c) N18Y/N21H/E96H/T100V/R104W or R104A; (d) N18Y/D25A or D25K/N92Q/T100D/R104W or R104A; (e) N18Y/D12A or D25K/N92Q/T100D/R104W or R104A; and (f) N18Y/D25A or D25K/N92Q/E96A/T100D/R104W or R104A.

In some embodiments, the nucleic acid molecules of the disclosure include a nucleotide sequence encoding a polypeptide that includes an amino acid sequence having at least 70%, 75%, 80%, 85%, 90%, 91%, 92%, 93%, 94%, 95%, 96%, 97%, 98%, or 99% sequence identity to SEQ ID NO: 1 or SEQ ID NO: 16, and further include the amino acid substitutions corresponding to the following amino acid substitutions: (a) N18Y/N92Q/T100D/R104W; (b) N18Y/N21H/N92Q/E96D/T100V/R104W; (c) N18Y/N21H/E96H/T100V/R104W; (d) N18Y/D25A/N92Q/T100D/R104W; (e) N18YID25KIN92Q/T100DIR104W; and (f) N18Y/D25A/N92Q/E96A/T100D/R104W. In some embodiments, the nucleic acid molecules of the disclosure include a nucleotide sequence encoding a polypeptide that includes an amino acid sequence having at least 70%, 75%, 80%, 85%, 90%, 91%, 92%, 93%, 94%, 95%, 96%, 97%, 98%, or 99% sequence identity to SEQ ID NO: 1 or SEQ ID NO: 16, and further include the amino acid substitutions corresponding to the following amino acid substitutions: (a) N18Y/N92Q/T100D/R104A; (b) N18Y/N21H/N92Q/E96D/T100V/R104A; (c) N18Y/N21H/E96H/T100V/R104A; (d) N18Y/D25A/N92Q/T100D/R104A; (e) N18Y/D25K/N92Q/T100D/R104A; and (f) N18Y/D25A/N92Q/E96A/T100D/R104A.

In some embodiments, the nucleic acid molecules of the disclosure include a nucleotide sequence encoding a polypeptide that includes an amino acid sequence having at least 75%, at least 80%, at least 85%, at least 90%, at least 95%, at least 96%, at least 97%, or at least 98%, at least 99% sequence identity to SEQ ID NO: 1 or SEQ ID NO: 16, and further include the amino acid substitutions corresponding to the following amino acid substitutions: (a) N18Y/N92Q/T100D/R104W; and (b) N21A. In some embodiments, the nucleic acid molecules of the disclosure include a nucleotide sequence encoding a polypeptide that includes an amino acid sequence having at least 75%, at least 80%, at least 85%, at least 90%, at least 95%, at least 96%, at least 97%, or at least 98%, at least 99% sequence identity to SEQ ID NO: 1 or SEQ ID NO: 16, and further include the amino acid substitutions corresponding to the following amino acid substitutions: (a) N18Y/N92Q/T100D/R104A; and (b) N21A. In some embodiments, the nucleic acid molecules of the disclosure include a nucleotide sequence encoding a polypeptide that includes an amino acid sequence having at least 75%, at least 80%, at least 85%, at least 90%, at least 95%, at least 96%, at least 97%, or at least 98%, at least 99% sequence identity to SEQ ID NO: 1 or SEQ ID NO: 16, and further include the amino acid substitutions corresponding to the following amino acid substitutions: (a) N18Y/N92Q/T100D/R104W; and (b) one or more mutations selected from the group consisting of D25A, D25K, and D25A/E96A. In some embodiments, the nucleic acid molecules of the disclosure include a nucleotide sequence encoding a polypeptide that includes an amino acid sequence having at least 75%, at least 80%, at least 85%, at least 90%, at least 95%, at least 96%, at least 97%, or at least 98%, at least 99% sequence identity to SEQ ID NO: 1 or SEQ ID NO: 16, and further include the amino acid substitutions corresponding to the following amino acid substitutions: (a) N18Y/N92Q/T100D/R104A; and (b) one or more mutations selected from the group consisting of D25A, D25K, and D25A/E96A. In some embodiments, the nucleic acid molecules of the disclosure include a nucleotide sequence encoding a polypeptide that includes an amino acid sequence having at least 75%, at least 80%, at least 85%, at least 90%, at least 95%, at least 96%, at least 97%, or at least 98%, at least 99% sequence identity to SEQ ID NO: 1 or SEQ ID NO: 16, and further include the amino acid substitutions corresponding to the following amino acid substitutions: (a) N18Y/N92Q/T100D/R104W; (b) N18Y/N21H/N92Q/E96D/T100V/R104W; (c) N18Y/N21H/E96H/T100V/R104W; (d) N18Y/D25A/N92Q/T100D/R104W; (e) N18Y/D25K/N92Q/T100D/R104W; and (f) N18Y/D25A/N92Q/E96A/T100D/R104W. In some embodiments, the nucleic acid molecules of the disclosure include a nucleotide sequence encoding a polypeptide that includes an amino acid sequence having at least 75%, at least 80%, at least 85%, at least 90%, at least 95%, at least 96%, at least 97%, or at least 98%, at least 99% sequence identity to SEQ ID NO: 1 or SEQ ID NO: 16, and further include the amino acid substitutions corresponding to the following amino acid substitutions: (a) N18Y/N92Q/T100D/R104A; (b) N18Y/N21H/N92Q/E96D/T100V/R104A; (c) N18Y/N21H/E96H/T100V/R104A; (d) N18Y/D25A/N92Q/T100D/R104A; (e) N18Y/D25K/N92Q/T100D/R104A; and (f) N18Y/D25A/N92Q/E96A/T100D/R104A. In some embodiments, the nucleic acid molecules of the disclosure include a nucleotide sequence encoding a polypeptide that includes an amino acid sequence having 100% sequence identity to SEQ ID NO: 1 or SEQ ID NO: 16, and further include the amino acid substitutions corresponding to the following amino acid substitutions: (a) N18Y/N92Q/T100D/R104W; (b) N18Y/N21H/N92Q/E96D/T100V/R104W; (c) N18Y/N21H/E96H/T100V/R104W; (d) N18Y/D25A/N92Q/T100D/R104W; (e) N18Y/D25K/N92Q/T100D/R104W; and (f) N18Y/D25A/N92Q/E96A/T100D/R104W. In some embodiments, the nucleic acid molecules of the disclosure include a nucleotide sequence encoding a polypeptide that includes an amino acid sequence having 100% sequence identity to SEQ ID NO: 1 or SEQ ID NO: 16, and further include the amino acid substitutions: a) N18Y/N21H/N92Q/E96D/T100V/R104W; and (b) N21A. In some embodiments, the nucleic acid molecules of the disclosure include a nucleotide sequence encoding a polypeptide that includes an amino acid sequence having 100% sequence identity to SEQ ID NO: 1 or SEQ ID NO: 16, and further include the amino acid substitutions: a) N18Y/N21H/N92Q/E96D/T100V/R104W; and (b) one or more mutations selected from the group consisting of D25A, D25K, and D25A/E96A. In some embodiments, the nucleic acid molecules of the disclosure include a nucleotide sequence encoding a polypeptide that includes an amino acid sequence having 100% sequence identity to SEQ ID NO: 1 or SEQ ID NO: 16, and further include the amino acid substitutions: a) N18Y/N21H/N92Q/E96D/T100V/R104A; and (b) N21A. In some embodiments, the nucleic acid molecules of the disclosure include a nucleotide sequence encoding a polypeptide that includes an amino acid sequence having 100% sequence identity to SEQ ID NO: 1 or SEQ ID NO: 16, and further include the amino acid substitutions: a) N18Y/N21H/N92Q/E96D/T100V/R104A; and (b) one or more mutations selected from the group consisting of D25A, D25K, and D25A/E96A.

In some embodiments disclosed herein, the nucleic acid molecules of the disclosure include a nucleotide sequence encoding a polypeptide which includes an amino acid sequence having at least 90%, 95%, 96%, 97, 98%, 99% sequence identity to an amino acid sequence selected from the group consisting of SEQ ID NOS: 2-15. In some embodiments, the nucleic acid molecules of the disclosure include a nucleotide sequence encoding a polypeptide which includes an amino acid sequence having at least 90%, 95%, 96%, 97, 98%, 99%, or 100% sequence identity to the amino acid sequence of SEQ ID NO: 2 or SEQ ID NO: 17. In some embodiments, the nucleic acid molecules include a nucleotide sequence encoding a polypeptide which includes an amino acid sequence having at least 90%, 95%, 96%, 97, 98%, 99%, or 100% sequence identity to the amino acid sequence of SEQ ID NO: 3 or SEQ ID NO: 18. In some embodiments, the nucleic acid molecules include a nucleotide sequence encoding a polypeptide which includes an amino acid sequence having 90%, 95%, 96%, 97, 98%, 99%, or 100% sequence identity to the amino acid sequence of SEQ ID NO: 4 or SEQ ID NO: 19. In some embodiments, the nucleic acid molecules include a nucleotide sequence encoding a polypeptide which includes an amino acid sequence having 90%, 95%, 96%, 97, 98%, 99%, or 100% sequence identity to the amino acid sequence of SEQ ID NO: 5 or SEQ ID NO: 20. In some embodiments, the nucleic acid molecules include a nucleotide sequence encoding a polypeptide which includes an amino acid sequence having 90%, 95%, 96%, 97, 98%, 99%, or 100% sequence identity to the amino acid sequence of SEQ ID NO: 6 or SEQ ID NO: 21. In some embodiments, the nucleic acid molecules include a nucleotide sequence encoding a polypeptide which includes an amino acid sequence having 90%, 95%, 96%, 97, 98%, 99%, or 100% sequence identity to the amino acid sequence of SEQ ID NO: 7 or SEQ ID NO: 22.

In some embodiments, the nucleic acid molecules of the disclosure include a nucleotide sequence encoding a polypeptide which includes an amino acid sequence having 90%, 95%, 96%, 97, 98%, 99%, or 100% sequence identity to the amino acid sequence of SEQ ID NO: 8 or SEQ ID NO: 23. In some embodiments, the nucleic acid molecules include a nucleotide sequence encoding a polypeptide which includes an amino acid sequence having 90%, 95%, 96%, 97, 98%, 99%, or 100% sequence identity to the amino acid sequence of SEQ ID NO: 9 or SEQ ID NO: 24. In some embodiments, the nucleic acid molecules include a nucleotide sequence encoding a polypeptide which includes an amino acid sequence having 90%, 95%, 96%, 97, 98%, 99%, or 100% sequence identity to the amino acid sequence of SEQ ID NO: 10 or SEQ ID NO: 25. In some embodiments, the nucleic acid molecules include a nucleotide sequence encoding a polypeptide which includes an amino acid sequence having 90%, 95%, 96%, 97, 98%, 99%, or 100% sequence identity to the amino acid sequence of SEQ ID NO: 11 or SEQ ID NO: 26. In some embodiments, the nucleic acid molecules include a nucleotide sequence encoding a polypeptide which includes an amino acid sequence having at least 90%, 95%, 96%, 97, 98%, 99%, or 100% sequence identity to the amino acid sequence of SEQ ID NO: 12 or SEQ ID NO: 27. In some embodiments, the nucleic acid molecules include a nucleotide sequence encoding a polypeptide which includes an amino acid sequence having at least 90%, 95%, 96%, 97, 98%, 99%, or 100% sequence identity to the amino acid sequence of SEQ ID NO: 13 or SEQ ID NO: 28. In some embodiments, the nucleic acid molecules include a nucleotide sequence encoding a polypeptide which includes an amino acid sequence having 90%, 95%, 96%, 97, 98%, 99%, or 100% sequence identity to the amino acid sequence of SEQ ID NO: 14 or SEQ ID NO: 29. In some embodiments, the nucleic acid molecules include a nucleotide sequence encoding a polypeptide which includes an amino acid sequence having 90%, 95%, 96%, 97, 98%, 99%, or 100% sequence identity to the amino acid sequence of SEQ ID NO: 15 or SEQ ID NO: 30.

The amino acid sequences of the corresponding precursor polypeptides of SEQ ID NOS: 1-15 are provided in SEQ ID NOS: 16-30 of the Sequence Listing.

In some embodiments, the nucleotide sequence is incorporated into an expression cassette or an expression vector. It will be understood that an expression cassette generally includes a construct of genetic material that contains coding sequences and enough regulatory information to direct proper transcription and/or translation of the coding sequences in a recipient cell, in vivo and/or ex vivo. Generally, the expression cassette may be inserted into a vector for targeting to a desired host cell and/or into an individual. As such, in some embodiments, an expression cassette of the disclosure include a coding sequence for the recombinant polypeptide as disclosed herein, which is operably linked to expression control elements, such as a promoter, and optionally, any or a combination of other nucleic acid sequences that affect the transcription or translation of the coding sequence.

In some embodiments, the nucleotide sequence is incorporated into an expression vector. It will be understood by one skilled in the art that the term “vector” generally refers to a recombinant polynucleotide construct designed for transfer between host cells, and that may be used for the purpose of transformation, e.g., the introduction of heterologous DNA into a host cell. As such, in some embodiments, the vector can be a replicon, such as a plasmid, phage, or cosmid, into which another DNA segment may be inserted so as to bring about the replication of the inserted segment. In some embodiments, the expression vector can be an integrating vector.

In some embodiments, the expression vector can be a viral vector. As will be appreciated by one of skill in the art, the term “viral vector” is widely used to refer either to a nucleic acid molecule (e.g., a transfer plasmid) that includes virus-derived nucleic acid elements that typically facilitate transfer of the nucleic acid molecule or integration into the genome of a cell or to a viral particle that mediates nucleic acid transfer. Viral particles will typically include various viral components and sometimes also host cell components in addition to nucleic acid(s). The term viral vector may refer either to a virus or viral particle capable of transferring a nucleic acid into a cell or to the transferred nucleic acid itself. Viral vectors and transfer plasmids contain structural and/or functional genetic elements that are primarily derived from a virus. In some embodiments, the viral vector is a bacculoviral vector, a retroviral vector, or a lentiviral vector. The term “retroviral vector” refers to a viral vector or plasmid containing structural and functional genetic elements, or portions thereof, that are primarily derived from a retrovirus. The term “lentiviral vector” refers to a viral vector or plasmid containing structural and functional genetic elements, or portions thereof, including LTRs that are primarily derived from a lentivirus, which is a genus of retrovirus.

Accordingly, also provided herein are vectors, plasmids, or viruses containing one or more of the nucleic acid molecules encoding any recombinant polypeptide or IL-10 polypeptide variant disclosed herein. The nucleic acid molecules can be contained within a vector that is capable of directing their expression in, for example, a cell that has been transformed/transduced with the vector. Suitable vectors for use in eukaryotic and prokaryotic cells are known in the art and are commercially available, or readily prepared by a skilled artisan.

DNA vectors can be introduced into eukaryotic cells via conventional transformation or transfection techniques. Suitable methods for transforming or transfecting host cells can be found in Sambrook et al. (2012, supra) and other standard molecular biology laboratory manuals, such as, calcium phosphate transfection, DEAE-dextran mediated transfection, transfection, microinjection, cationic lipid-mediated transfection, electroporation, transduction, scrape loading, ballistic introduction, nucleoporation, hydrodynamic shock, and infection.

Viral vectors that can be used in the disclosure include, for example, baculoviral vectors, retrovirus vectors, adenovirus vectors, and adeno-associated virus vectors, lentivirus vectors, herpes virus, simian virus 40 (SV40), and bovine papilloma virus vectors (see, for example, Gluzman (Ed.), Eukaryotic Viral Vectors, CSH Laboratory Press, Cold Spring Harbor, N.Y.).

The precise components of the expression system are not critical. For example, a recombinant polypeptide as disclosed herein can be produced in a eukaryotic host, such as a mammalian cells (e.g., COS cells, NIH 3T3 cells, or HeLa cells). These cells are available from many sources, including the American Type Culture Collection (Manassas, VA). In selecting an expression system, care should be taken to ensure that the components are compatible with one another. Artisans or ordinary skill are able to make such a determination. Furthermore, if guidance is required in selecting an expression system, skilled artisans may consult P. Jones, “Vectors: Cloning Applications”, John Wiley and Sons, New York, N.Y., 2009).

The nucleic acid molecules provided can contain naturally occurring sequences, or sequences that differ from those that occur naturally, but, due to the degeneracy of the genetic code, encode the same polypeptide, e.g., antibody. These nucleic acid molecules can consist of RNA or DNA (for example, genomic DNA, cDNA, or synthetic DNA, such as that produced by phosphoamidite-based synthesis), or combinations or modifications of the nucleotides within these types of nucleic acids. In addition, the nucleic acid molecules can be double-stranded or single-stranded (e.g., either a sense or an antisense strand).

The nucleic acid molecules are not limited to sequences that encode polypeptides (e.g., IL-10 polypeptide variants); some or all of the non-coding sequences that lie upstream or downstream from a coding sequence (e.g., the coding sequence of an IL-10 polypeptide variant) can also be included. Those of ordinary skill in the art of molecular biology are familiar with routine procedures for isolating nucleic acid molecules. They can, for example, be generated by treatment of genomic DNA with restriction endonucleases, or by performance of the polymerase chain reaction (PCR). In the event the nucleic acid molecule is a ribonucleic acid (RNA), molecules can be produced, for example, by in vitro transcription.

In another aspect, provided herein are cell cultures including at least one recombinant cell as disclosed herein, and a culture medium. Generally, the culture medium can be any suitable culture medium for culturing the cells described herein. Techniques for transforming a wide variety of the above-mentioned host cells and species are known in the art and described in the technical and scientific literature. Accordingly, cell cultures including at least one recombinant cell as disclosed herein are also within the scope of this application. Methods and systems suitable for generating and maintaining cell cultures are known in the art.

### C. Recombinant Cells and Cell Cultures

The recombinant nucleic acids of the present disclosure can be introduced into a host cell, such as, for example, a human T lymphocyte, to produce a recombinant cell containing the nucleic acid molecule. Introduction of the nucleic acid molecules of the disclosure into cells can be achieved by methods known to those skilled in the art such as, for example, viral infection, transfection, conjugation, protoplast fusion, lipofection, electroporation, nucleofection, calcium phosphate precipitation, polyethyleneimine (PEI)-mediated transfection, DEAE-dextran mediated transfection, liposome-mediated transfection, particle gun technology, calcium phosphate precipitation, direct micro-injection, nanoparticle-mediated nucleic acid delivery, and the like.

Accordingly, in some embodiments, the nucleic acid molecules can be delivered by viral or non-viral delivery vehicles known in the art. For example, the nucleic acid molecule can be stably integrated in the host genome, or can be episomally replicating, or present in the recombinant host cell as a mini-circle expression vector for transient expression. Accordingly, in some embodiments, the nucleic acid molecule is maintained and replicated in the recombinant host cell as an episomal unit. In some embodiments, the nucleic acid molecule is stably integrated into the genome of the recombinant cell. Stable integration can be achieved using classical random genomic recombination techniques or with more precise techniques such as guide RNA-directed CRISPR/Cas9 genome editing, or DNA-guided endonuclease genome editing with NgAgo (Natronobacterium gregoryi Argonaute), or TALENs genome editing (transcription activator-like effector nucleases). In some embodiments, the nucleic acid molecule is present in the recombinant host cell as a mini-circle expression vector for transient expression.

The nucleic acid molecules can be encapsulated in a viral capsid or a lipid nanoparticle, or can be delivered by viral or non-viral delivery means and methods known in the art, such as electroporation. For example, introduction of nucleic acids into cells may be achieved by viral transduction. In a non-limiting example, baculoviral virus or adeno-associated virus (AAV) can be engineered to deliver nucleic acids to target cells via viral transduction. Several AAV serotypes have been described, and all of the known serotypes can infect cells from multiple diverse tissue types. AAV is capable of transducing a wide range of species and tissues in vivo with no evidence of toxicity, and it generates relatively mild innate and adaptive immune responses.

Lentiviral-derived vector systems are also useful for nucleic acid delivery and gene therapy via viral transduction. Lentiviral vectors offer several attractive properties as gene-delivery vehicles, including: (i) sustained gene delivery through stable vector integration into host genome; (ii) the capability of infecting both dividing and non-dividing cells; (iii) broad tissue tropisms, including important gene- and cell-therapy-target cell types; (iv) no expression of viral proteins after vector transduction; (v) the ability to deliver complex genetic elements, such as polycistronic or intron-containing sequences; (vi) a potentially safer integration site profile; and (vii) a relatively easy system for vector manipulation and production.

In some embodiments, host cells can be genetically engineered (e.g., transduced or transformed or transfected) with, for example, a vector construct of the present application that can be, for example, a viral vector or a vector for homologous recombination that includes nucleic acid sequences homologous to a portion of the genome of the host cell, or can be an expression vector for the expression of the polypeptides of interest. Host cells can be either untransformed cells or cells that have already been transfected with at least one nucleic acid molecule.

In some embodiments, the recombinant cell is a prokaryotic cell or a eukaryotic cell. In some embodiments, the cell is in vivo. In some embodiments, the cell is ex vivo. In some embodiments, the cell is in vitro. In some embodiments, the recombinant cell is a eukaryotic cell. In some embodiments, the recombinant cell is an animal cell. In some embodiments, the animal cell is a mammalian cell. In some embodiments, the animal cell is a human cell. In some embodiments, the cell is a non-human primate cell. In some embodiments, the recombinant cell is an immune system cell, e.g., a lymphocyte (e.g., a T cell or NK cell), or a dendritic cell. In some embodiments, the immune cell is a B cell, a monocyte, a natural killer (NK) cell, a basophil, an eosinophil, a neutrophil, a dendritic cell, a macrophage, a regulatory T cell, a helper T cell (T_{H}), a cytotoxic T cell (T_{CTL}), or other T cell. In some embodiments, the immune system cell is a T lymphocyte. In some embodiments, the cell can be obtained by leukapheresis performed on a sample obtained from a subject. In some embodiments, the subject is a human patient. Non-limiting examples of suitable cell lines include Spodoptera frugiperda (SfP), Trichoplusia ni (Hi5) cells, Expi-293F cells, Expi-CHO cells, and HEK-293T (ATCC CRL-3216),

In another aspect, provided herein are cell cultures including at least one recombinant cell as disclosed herein, and a culture medium. Generally, the culture medium can be any suitable culture medium for culturing the cells described herein. Techniques for transforming a wide variety of the above-mentioned host cells and species are known in the art and described in the technical and scientific literature. Accordingly, cell cultures including at least one recombinant cell as disclosed herein are also within the scope of this application. Methods and systems suitable for generating and maintaining cell cultures are known in the art.

### D. Methods for Producing an IL-10 Polypeptide

In another aspect, some embodiments of the disclosure relate to various methods for producing a recombinant polypeptide of the disclosure, the methods include: (a) providing one or more recombinant cells of the disclosure; and culturing the recombinant cell(s) in a culture medium such that the cells produce the polypeptide encoded by the recombinant nucleic acid molecule. Accordingly, the recombinant polypeptides produced by the method disclosed herein are also within the scope of the disclosure.

Non-limiting exemplary embodiments of the disclosed methods for producing a recombinant polypeptide can include one or more of the following features. In some embodiments, the methods further include isolating and/or purifying the produced polypeptide. In some embodiments, the methods for producing a recombinant polypeptide of the disclosure further include isolating and/or purifying the produced polypeptide. In some embodiments, the methods for producing a polypeptide of the disclosure further include structurally modifying the produced polypeptide to increase half-life.

In some embodiments, the modification includes one or more alterations selected from the group consisting of fusion to a human Fc antibody fragment, fusion to albumin, and PEGylation. For example, any of the recombinant polypeptides disclosed herein can be prepared as fusions or chimeric polypeptides that include a recombinant polypeptide and a heterologous polypeptide (e.g., a polypeptide that is not IL-10 or a variant thereof). Exemplary heterologous polypeptides can increase the circulating half-life of the recombinant polypeptide in vivo, and may, therefore, further enhance the properties of the recombinant polypeptides of the disclosure. In various embodiments, the heterologous polypeptide that increases the circulating half-life may be a serum albumin, such as human serum albumin, or the Fc region of the IgG subclass of antibodies that lacks the IgG heavy chain variable region. Exemplary Fc regions can include a mutation that inhibits complement fixation and Fc receptor binding, or it may be lytic, e.g., able to bind complement or to lyse cells via another mechanism, such as antibody-dependent complement lysis (ADCC).

In some embodiments, the “Fc region” can be a naturally occurring or synthetic polypeptide that is homologous to the IgG C-terminal domain produced by digestion of IgG with papain. IgG Fc has a molecular weight of approximately 50 kDa. The recombinant fusion polypeptides of the disclosure can include the entire Fc region, or a smaller portion thereof that retains the ability to extend the circulating half-life of a fusion polypeptide of which it is a part. In addition, full-length or fragmented Fc regions can be variants of the wild-type molecule. That is, they can contain mutations that may or may not affect the function of the polypeptides; as described further below, native activity is not necessary or desired in all cases. In some embodiments, the recombinant fusion protein (e.g., an IL-10 partial agonist or antagonist as described herein) includes an IgG1, IgG2, IgG3, or IgG4 Fc region.

The Fc region can be “lytic” or “non-lytic”, but is typically non-lytic. A non-lytic Fc region typically lacks a high affinity Fc receptor binding site and a C′1q binding site. The high affinity Fc receptor binding site of murine IgG Fc includes the Leu residue at position 235 of IgG Fc. Thus, the Fc receptor binding site can be destroyed by mutating or deleting Leu 235. For example, substitution of Glu for Leu 235 inhibits the ability of the Fc region to bind the high affinity Fc receptor. The murine C′1q binding site can be functionally destroyed by mutating or deleting the Glu 318, Lys 320, and Lys 322 residues of IgG. For example, substitution of Ala residues for Glu 318, Lys 320, and Lys 322 renders IgG1 Fc unable to direct antibody-dependent complement lysis. In contrast, a lytic IgG Fc region has a high affinity Fe receptor binding site and a C′1q binding site. The high affinity Fc receptor binding site includes the Leu residue at position 235 of IgG Fc, and the C′1q binding site includes the Glu 318, Lys 320, and Lys 322 residues of IgG1. Lytic IgG Fc has wild-type residues or conservative amino acid substitutions at these sites. Lytic IgG Fc can target cells for antibody dependent cellular cytotoxicity or complement directed cytolysis (CDC). Appropriate mutations for human IgG are also known in the art.

In other embodiments, the recombinant fusion polypeptide can include a recombinant IL-10 polypeptide of the disclosure and a polypeptide that functions as an antigenic tag, such as a FLAG sequence. FLAG sequences are recognized by biotinylated, highly specific, anti-FLAG antibodies. In some embodiments, the recombinant fusion polypeptide further includes a C-terminal c-myc epitope tag.

In other embodiments, the recombinant fusion polypeptide includes a recombinant IL-10 polypeptide of the disclosure and a heterologous polypeptide that functions to enhance expression or direct cellular localization of the IL-10 polypeptide, such as the Aga2p agglutinin subunit.

In other embodiments, a fusion polypeptide including a recombinant IL-10 polypeptide of the disclosure and an antibody or antigen-binding portion thereof can be generated. The antibody or antigen-binding component of the fusion recombinant IL-10 polypeptide can serve as a targeting moiety. For example, it can be used to localize the recombinant IL-10 polypeptide to a particular subset of cells or target molecule. Methods of generating cytokine-antibody chimeric polypeptides are known in the art.

In some embodiments, the recombinant IL-10 polypeptides of the disclosure can be modified with one or more polyethylene glycol (PEG) molecules to increase its half-life. The term “PEG” as used herein means a polyethylene glycol molecule. In its typical form, PEG is a linear polymer with terminal hydroxyl groups and has the formula HO-CH₂CH₂-(CH₂CH₂O)n-CH₂CH₂-OH, where n is from about 8 to about 4000.

Generally, n is not a discrete value but constitutes a range with approximately Gaussian distribution around an average value. The terminal hydrogen may be substituted with a capping group such as an alkyl or alkanol group. PEG can have at least one hydroxy group, more preferably it is a terminal hydroxy group. This hydroxy group is can be attached to a linker moiety which can react with the peptide to form a covalent linkage. Numerous derivatives of PEG exist in the art. The PEG molecule covalently attached to the recombinant IL-10 polypeptides of the present disclosure may be approximately 10,000, 20,000, 30,000, or 40,000 daltons average molecular weight. PEGylation reagents may be linear or branched molecules and may be present singularly or in tandem. The PEGylated IL-10 polypeptides of the present disclosure can have tandem PEG molecules attached to the C-terminus and/or the N-terminus of the peptide. The term “PEGylation” as used herein means the covalent attachment of one or more PEG molecules, as described above, to a molecule such as the IL-10 polypeptides of the present disclosure.

### E. Pharmaceutical Compositions

The recombinant polypeptides, nucleic acids, recombinant cells, and/or cell cultures of the disclosure can be incorporated into compositions, including pharmaceutical compositions. Such compositions generally include the recombinant polypeptides, nucleic acids, recombinant cells, and/or cell cultures as described herein and a pharmaceutically acceptable excipient, e.g., carrier.

Accordingly, one aspect of the present disclosure relates to pharmaceutical compositions that include a pharmaceutical acceptable carrier and one or more of the following: (a) a recombinant polypeptide of the disclosure; (b) a recombinant nucleic acid of the disclosure; and (c) a pharmaceutically acceptable carrier.

In some embodiments, the pharmaceutical compositions of the disclosure are formulated for the treating, preventing, ameliorating a disease such as cancer, or for reducing or delaying the onset of the disease.

Non-limiting exemplary embodiments of the disclosed pharmaceutical compositions can include one or more of the following features. In some embodiments, the composition includes a recombinant polypeptide of the disclosure and a pharmaceutically acceptable carrier. In some embodiments, the composition includes a recombinant cell of the disclosure and a pharmaceutically acceptable carrier. In some embodiments, the recombinant cell expresses a recombinant polypeptide of the disclosure. Examples of recombinant cells genetically modified to express and secret interleukin as new therapeutic approaches are described previously in, for example, Steidler L. et al., Nature Biotechnology, Vol. 21, No. 7, July 2003 and Oh J.H et al., mSphere, Vol. 5, Issue 3, May/June 2020.

In some embodiments, the composition includes a recombinant nucleic acid of the disclosure and a pharmaceutically acceptable carrier. In some embodiments, the recombinant nucleic acid is encapsulated in a viral capsid or a lipid nanoparticle.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™. (BASF, Parsippany, N.J.), or phosphate buffered saline (PBS). In all cases, the composition should be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants, e.g., sodium dodecyl sulfate. Prevention of the action of microorganisms can be achieved by various antibacterial and antifungal agents, for example, parabens, chlorobutanol, phenol, ascorbic acid, thimerosal, and the like. In many cases, it will be generally to include isotonic agents, for example, sugars, polyalcohols such as mannitol, sorbitol, and/or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Systemic administration of the subject recombinant polypeptides of the disclosure can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art

In some embodiments, the recombinant polypeptides of the disclosure can also be administered by transfection or infection using methods known in the art, including but not limited to the methods described in McCaffrey et al. (Nature 418:6893, 2002), Xia et al. (Nature Biotechnol. 20: 1006-1010, 2002), or Putnam (Am. J. Health Syst. Pharm. 53: 151-160, 1996, erratum at Am. J. Health Syst. Pharm. 53:325, 1996).

In some embodiments, the subject recombinant polypeptides of the disclosure are prepared with carriers that will protect the recombinant polypeptides against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811. As described in greater detail below, the recombinant polypeptides of the present disclosure may also be modified to achieve extended duration of action such as by PEGylation, acylation, Fc fusions, linkage to molecules such as albumin, etc. In some embodiments, the recombinant polypeptides can be further modified to prolong their half-life in vivo and/or ex vivo. Non-limiting examples of known strategies and methodologies suitable for modifying the recombinant polypeptides of the disclosure include (1) chemical modification of a recombinant polypeptide described herein with highly soluble macromolecules such as polyethylene glycol (“PEG”) which prevents the recombinant polypeptides from contacting with proteases; and (2) covalently linking or conjugating a recombinant polypeptide described herein with a stable protein such as, for example, albumin. Accordingly, in some embodiments, the recombinant polypeptides of the disclosure can be fused to a stable protein, such as, albumin. For example, human albumin is known as one of the most effective proteins for enhancing the stability of polypeptides fused thereto and there are many such fusion proteins reported.

In some embodiments, the pharmaceutical compositions of the disclosure include one or more pegylation reagents. As used herein, the term “PEGylation” refers to modifying a protein by covalently attaching polyethylene glycol (PEG) to the protein, with “PEGylated” referring to a protein having a PEG attached. A range of PEG, or PEG derivative sizes with optional ranges of from about 10,000 Daltons to about 40,000 Daltons may be attached to the recombinant polypeptides of the disclosure using a variety of chemistries. In some embodiments, the average molecular weight of said PEG, or PEG derivative, is about 1 kD to about 200 kD such as, e.g., about 10 kD to about 150 kD, about 50 kD to about 100 kD, about 5 kD to about 100 kD, about 20 kD to about 80 kD, about 30 kD to about 70 kD, about 40 kD to about 60 kD, about 50 kD to about 100 kD, about 100 kD to about 200 kD, or about 150 kD to about 200 kD. In some embodiments, the average molecular weight of said PEG, or PEG derivative, is about 5 kD, about 10 kD, about 20 kD, about 30 kD, about 40 kD, about 50 kD, about 60 kD, about 70 kD, or about 80 kD. In some embodiments, the average molecular weight of said PEG, or PEG derivative, is about 40 kD. In some embodiments, the pegylation reagent is selected from methoxy polyethylene glycol-succinimidyl propionate (mPEG-SPA), mPEG-succinimidyl butyrate (mPEG-SBA), mPEG-succinimidyl succinate (mPEG-SS), mPEG-succinimidyl carbonate (mPEG-SC), mPEG-Succinimidyl Glutarate (mPEG-SG), mPEG-N-hydroxyl-succinimide (mPEG-NHS), mPEG-tresylate and mPEG-aldehyde. In some embodiments, the pegylation reagent is polyethylene glycol; for example said pegylation reagent is polyethylene glycol with an average molecular weight of 20,000 Daltons covalently bound to the N-terminal methionine residue of the recombinant polypeptides of the disclosure. In some embodiments, the pegylation reagent is polyethylene glycol with an average molecular weight of about 5 kD, about 10 kD, about 20 kD, about 30 kD, about 40 kD, about 50 kD, about 60 kD, about 70 kD, or about 80 kD covalently bound to the N-terminal methionine residue of the recombinant polypeptides of the disclosure. In some embodiments, the pegylation reagent is polyethylene glycol with an average molecular weight of about 40 kD covalently bound to the N-terminal methionine residue of the recombinant polypeptides of the disclosure.

Accordingly, in some embodiments, the recombinant polypeptides of the disclosure are chemically modified with one or more polyethylene glycol moieties, e.g., PEGylated; or with similar modifications, e.g. PASylated. In some embodiments, the PEG molecule or PAS molecule is conjugated to one or more amino acid side chains of the disclosed recombinant polypeptide. In some embodiments, the PEGylated or PASylated polypeptide contains a PEG or PAS moiety on only one amino acid. In other embodiments, the PEGylated or PASylated polypeptide contains a PEG or PAS moiety on two or more amino acids, e.g., attached to two or more, five or more, ten or more, fifteen or more, or twenty or more different amino acid residues. In some embodiments, the PEG or PAS chain is 2000, greater than 2000, 5000, greater than 5,000, 10,000, greater than 10,000, greater than 10,000, 20,000, greater than 20,000, and 30,000 Da. The PASylated polypeptide may be coupled directly to PEG or PAS (e.g., without a linking group) through an amino group, a sulfhydryl group, a hydroxyl group, or a carboxyl group. In some embodiments, the recombinant polypeptide of the disclosure is covalently bound to a polyethylene glycol with an average molecular weight ranging from about 1 kD to about 200 kD such as, e.g., about 10 kD to about 150 kD, about 50 kD to about 100 kD, about 5 kD to about 100 kD, about 20 kD to about 80 kD, about 30 kD to about 70 kD, about 40 kD to about 60 kD, about 50 kD to about 100 kD, about 100 kD to about 200 kD, or about 150 kD to about 200 kD. In some embodiments, the recombinant polypeptide of the disclosure is covalently bound to a polyethylene glycol with an average molecular weight of about 5 kD, about 10 kD, about 20 kD, about 30 kD, about 40 kD, about 50 kD, about 60 kD, about 70 kD, or about 80 kD. In some embodiments, the recombinant polypeptide of the disclosure is covalently bound to a polyethylene glycol with an average molecular weight of about 40 kD.

### Incorporation of Site-Specific PEGylation Sites

In some embodiments, the recombinant polypeptides of the disclosure (e.g., IL-10 variants) may be modified by the incorporation of non-natural amino acids with non-naturally occurring amino acid side chains to facilitate site specific conjugation (e.g., PEGylation) as described in, for example, U.S. Pat. Nos. 7,045,337; 7,915,025; Dieters, et al. (2004) Bioorganic and Medicinal Chemistry Letters 14(23):5743-5745; Best, M (2009) Biochemistry 48(28): 6571-6584. In some embodiments, cysteine residues may be incorporated at various positions within the recombinant polypeptides of the disclosure to facilitate site-specific PEGylation via the cysteine side chain as described in, for example, Dozier and Distefano (2015) International Journal of Molecular Science 16(10): 25831-25864.

In certain embodiments, the present disclosure provides IL-10 variant polypeptides comprising incorporation of one or more amino acids enabling site specific PEGylation (e.g., cysteine or non-natural amino acid) of the present disclosure, wherein the amino acid substitution for site specific PEGylation site is not in the interface between the IL-10 and a component of the IL-10 receptor signaling complex, e.g., IL-10Rα or IL-10Rβ. In such instances, the incorporation of the site-specific amino acid modification are incorporated at IL-10 amino acid positions other than amino acid residues 34-69, 78-87, and 108-160 of SEQ ID NO: 1 or SEQ ID NO: 16, which encompass the IL-10Rβ binding interface revealed in the crystal structure described herein. Accordingly, in some embodiments, the present disclosure provides IL-10 variant polypeptides comprising site-specific amino acid substitutions to enable site specific conjugation (e.g., PEGylation) at one or more following amino acid positions 34-69, 78-87, and 108-160 of SEQ ID NO: 1 or SEQ ID NO: 16.

### Incorporation of Site-Specific PEGylation Sites

In some embodiments, the interaction of the IL-10 protein with the IL-10Rβ protein may be modulated by incorporation of site specific pegylation at the amino acid locations described herein at the IL-10 interface. The incorporation of non-natural amino acids (or cysteine residues) that facilitate site specific PEGylation at one or more of positions corresponding to an amino acid residue selected from the group consisting of 13-33, 70-77, and 88-107 of SEQ ID NO: 1 (i.e., residues 31-51, 88-95, 106-125 when numbered in accordance with the pre-protein human IL-10 protein including the signal peptide, i.e., the sequence of SEQ ID NO: 16) provide IL-10 variant polypeptides with modulated binding to the IL-10Rβ protein resulting in an variant IL-10/IL-10Rα/IL-10Rβ receptor complex having partial agonist activity. In such instances where PEG molecules are incorporated at the interface, so as to not completely disrupt the binding of the IL-10 variant polypeptide with the IL-10Rβ or IL10R1 proteins thereby ablating activity, the PEG is typically a low molecular weight PEG species of from about 1 kDa, alternatively about 2 kDa, alternatively about 3 kDa, alternatively about 4 kDa, alternatively about 5 kDa, alternatively about 6 kDa, alternatively about 7 kDa, alternatively about 8 kDa, alternatively about 9 kDa, alternatively about 10 kDa, alternatively about 12 kDa, alternatively about 15 kDa, or alternatively about 20 kDa.

### Formulations

The IL-10 variant polypeptides of the present disclosure are useful for the treatment and/or prevention of inflammatory and/or autoimmune diseases in a mammalian subject suffering therefrom by the administration to the subject of a therapeutically effective amount of such IL-10 variant polypeptide, alone or in combination with one or more additional therapeutic agents. Additionally, the present disclosure provides a method of treating and/or preventing an inflammatory diseases in a mammalian subject suffering therefrom by the administration to the subject of a therapeutically effective amount of such IL-10 variant polypeptide, alone or in combination with one or more additional therapeutic agents.

In some embodiments, the compositions and methods of the present disclosure are useful in the treatment of inflammatory diseases of the gastrointestinal (GI) tract including but not limited to Crohn’s Disease (CD), ulcerative colitis (UC), and other forms of inflammatory bowel disease (IBD) which are characterized by chronic inflammation of the intestinal tract. While Crohn’s disease may affect areas of the intestinal tract from the mouth to the anus, ulcerative colitis is typically associated with inflammation of the large intestine and rectum.

Traditional first line treatments for such inflammatory intestinal conditions include 5-aminosalicyclic acid, optionally in combination with corticosteroids. The more recent development of therapeutic proteins such anti-TNF alpha antibodies (e.g., infliximab, adalimumab, golimumab) and integrin receptor antagonists (e.g., natalizumab and vedolizumab) and IL23 antagonists (e. g. Ustekinumab). While providing significant efficacy in the treatment of chronic inflammatory diseases, the conventional parenteral (IM, IV or SQ) administration of such polypeptide in systemic exposure with a comparatively small fraction of the agent being exposed to the therapeutic. Although these agents provide specific disruption of the inflammatory pathways, the systemic exposure to such agents resulting from their parenteral administration is associated with significant systemic side effects including immunosuppression leading to increased vulnerability to serious, potentially life-threatening, infections including tuberculosis, fungal infections, urinary tract infections, and lymphoma. Such significant systemic toxicities limit the dose of the agent to be administered resulting in suboptimal exposure of the affected tissue to the biologic therapeutic.

Although the selective nature of the IL-10 variant polypeptides of the present disclosure mitigate such systemic side effects when administered parenterally, in some instances it may be desirable to provide direct administration of the IL-10 variant polypeptides to the intestinal tract.

### Formulation for Oral Administration of IL-10 Variant Polypeptides

The present disclosure further provides a method of treating and/or preventing an inflammatory disease of the gastrointestinal tract in a mammalian subject, the method comprising administering to the mammalian subject a therapeutically or prophylactically effective amount of a composition comprising an IL-10 variant polypeptideof the present disclosure, alone or in combination with one or more additional therapeutic agents. The present disclosure further provides an enteral composition comprising an IL-10 variant polypeptide of the present disclosure. In some embodiments, the enteral composition comprises a pharmaceutically acceptable formulation of a IL-10 variant polypeptide wherein such formulation resists degradation of the IL-10 variant polypeptide in the upper GI tract and disintegrates in the lower GI tract thus facilitating administration of the IL-10 variant polypeptide to the lower GI tract including the small intestine, large intestine, rectum and anus and treatment of inflammatory diseases of the lower GI tract including but not limited to ulcerative colitis. Exemplary polypeptide formulations suitable for oral administration are known in the art such as those described in Hamman, et al “Oral Delivery of Peptide Drugs” (2005). BioDrugs 19, 165-177; Blichmann, P. “Oral Delivery of Peptide Drugs for Mitigation of Crohn’s Disease” (2012). All Theses. 1486 and reviewed in Anselmo, et al “Non-invasive delivery strategies for biologics) (2019) Nat Rev Drug Discov 18, 19-40.

### Delivery of Engineered Prokaryotic Cells

In another embodiment, the present disclosure provides methods for administration of an IL-10 variant to the GI tract of a mammalian subject the method comprising the step of administering to the subject a pharmaceutically acceptable composition comprising a recombinantly engineered prokaryotic cell, the prokaryotic cell comprising a nucleic acid sequence encoding an IL-10 variant of the present disclosure operably linked to one or more expression control sequences such that the IL-10 variant is expressed in the recombinant procaryotic cell and the IL-10 variant released into the GI tract by secretion from or lysis of the recombinantly modified procaryotic cell or displayed on the surface of the procaryotic cell. Examples of recombinantly modified bacterial cells for the administration of IL-10 are known in the art Steidler, et al. 2003, supra. In some embodiments, the engineered bacterial cell expressing the IL-10 variant may be administered orally, typically in aqueous suspension, or rectally (e.g. enema).

### Procaryotic Viral Delivery to Intestinal Flora

In one embodiment, the present disclosure provides a recombinantly modified procaryotic virus (e.g., bacteriophage) comprising a nucleic acid sequence encoding an IL-10 variant polypeptide. In another embodiment, the present disclosure provides a method of treating an inflammatory disease of the intestinal tract of a mammalian subject, the method comprising administering to the subject a therapeutically effective amount of a pharmaceutically acceptable formulation of a recombinantly modified procaryotic virus (e.g., bacteriophage) comprising a nucleic acid sequence encoding an IL-10 variant polypeptide, the nucleic acid sequence operably linked to one or more expression control sequences functional in a host procaryotic cell for the virus such that upon infection of a procaryotic cell by the procaryotic virus, the IL-10 variant is expressed in the host cell. In one embodiment, the bacteriophage is a lytic phage such that the bacteriophage induces the lytic pathway of the bacterial cell following infection resulting local release of the IL-10 variant and delivery of the IL-10 variant to the intestinal mucosa.

As used herein, the terms ‘procaryotic virus,” “bacteriophage” and “phage” are used interchangeably hereinto describe any of a variety of bacterial viruses that infect and replicate within a bacterium. A wide variety of bacteriophages capable of selection a broad range of bacterial cells have been identified and characterized extensively in the scientific literature. Since bacteriophages exhibit significant selectivity in the bacterial cell susceptible to infection, the bacteriophage is typically selected in view of the target bacterium of the intestinal flora for expression. Samples of bacteriophages suitable as starting material for the generation of the recombinantly engineered bacteriophage against any of a wide variety of bacterial cells are available from the American Type Culture Collection (Manassas, Va.). The manipulation of the phage genome is conducted in accordance with standard methodologies for the manipulation of recombinant nucleic acids well known to those of skill in the art such as those describing in Molecular Cloning: A Laboratory Manual (available from Cold Spring Harbor Press) and conventional reagents available from a wide variety of laboratory supply houses.

The delivery of the IL-10 variant to the site of infection is achieved by a bacteriophage capable of infecting the a procaryotic cell of the intestinal flora of the subject, the bacteriophage modified using recombinant technology to introduce an expression cassette comprising a promoter active in the target procaryotic cell operably linked to a nucleic acid sequence encoding an IL-10 variant, said expression cassette inserted into a non-essential region of a bacteriophage genome such that following transfection of the target bacterial cell by the recombinantly engineered bacteriophage, the IL-10 variant polypeptide is expressed in the target bacterial cell. For example, in the case of bacteriophage targeting S. aureus cells, the expression cassette would employ a S. aureus promoter driving expression of the IL-10 variant inserted into a non-essential region of the S. aureus specific bacteriophage. The identification of non-essential regions of the bacteriophage genome are readily identified by the known genomic organization and coding sequences of such phages. Prokaryotic promoter sequences active in a wide variety of bacteria are well known in the art. Promoter sequences may be obtained from excising naturally occurring sequences or through sequencing and synthetic synthesis of nucleic acid sequences corresponding to naturally occurring prokaryotic promoter sequences of the target bacterial cell. See, e.g. Estrem, et al. (1999) Bacterial promoter architecture: Subsite structure of UP elements and interactions with the carboxy-terminal domain of the RNA polymerase alpha subunit; Genes & Development 13(16): 2134-2147.

### Codon Optimization

The amino acid sequences and corresponding encoding nucleic acid sequences if IL-10 variant are provided herein and nucleic acid sequences may be generated by one of skill in the art based on the degeneracy of the genetic code. In one embodiment, the mammalian sequence of the IL-10 variant is optimized for expression in the bacterial cell target environment through the use of codons optimized for expression. The techniques for the construction of synthetic nucleic acid sequences encoding IL-10 variant using and preferred codons optimal for bacterial cell expression may be determined by computational methods analyzing the commonality of codon usage for encoding native proteins of the bacteriophage genome and their relative abundance by techniques well known in the art. The codon usage database (www.kazusa.or.jp/codon) may be used for generation of codon optimized sequences in bacterial environments. Furthermore, a variety of software tools are available to convert sequences from one organism to the optimal codon usage for a different host organism such as the JCat Codon Optimization Tool (www.jcat.de), Integrated DNA technologies Codon Optimization Tool (www.idtdna.com/CodonOpt) or the Optimizer online codon optimization tool (genomes.urv.es/OPTIMIZER). Such synthetic sequences may be constructed by techniques well known in the art for the construction of synthetic nucleic acid molecules and may be obtained from a variety of commercial vendors.

### Deletion of PAM Motifs of Phage Vector

In some embodiments, to facilitate expression of the IL-10 polypeptide from the recombinant vector in the intestinal flora, the recombinant procaryotic vector of the present invention may be modified to avoid or inhibit the defense mechanisms of the bacterial host cell. Bacterial hosts have developed defense mechanisms to guard against bacteriophage infection such as the Cas9 endonuclease which introduces a double strand DNA cleavage inactivating the phage. The Cas9 endonuclease surveys the genome to identify a protospacer adjacent motif (PAM) site which is essential for Cas9 to bind to the target DNA. As PAM sequences are essential for Cas9 function, elimination of Cas9 sequences from the procaryotic virus minimizes the ability of the Cas9 endonuclease endogenous to the subject’s intestinal bacterial flora to neutralize the invading phage encoding the IL-10 variant.

### Engineered Regulatory T Cells (Tregs) That Express IL-10 Variant Polypeptide

In some embodiments, the present disclosure further provides a recombinantly modified Treg cell, the Treg comprising an expression cassette comprising a nucleic acid sequence encoding an IL-10 variant, optionally comprising a signal peptide, operably linked to expression control sequences functional in the Treg cell capable of directing the transcription and translation of the nucleic acid sequence encoding the IL-10 variant, optionally in secreted form when associated with a signal peptide. One skilled in the art will understand that the term “regulatory T cell” or “Treg cell” refers to a type of CD4+ T cell that can suppress the responses of other T cells including but not limited to effector T cells (Teff). Treg cells are characterized by expression of CD4, the α-subunit of the IL-2 receptor (CD25), and the transcription factor forkhead box P3 (FOXP3). Engineered Treg cells and the process for their preparation are well known in the art. See, e.g. McGovern, et al. Engineering Specificity and Function of Therapeutic Regulatory T Cells (2017). Front. Immunol. 8:1517; Scott, D. Genetic Engineering of T Cells for Immune Tolerance (2020) Molecular Therapy: Methods & Clinical Development 16:103 and include CAR Tregs as described in Zhang, et al (2018) Frontiers in Immunology 12(9):235.

### Administration of IL-10 via Viral Vector

In some embodiments, the IL-10 variant may be administered to the mammalian subject by contacting the subject with an expression vector comprising a nucleic acid sequence encoding the IL-10 variant. Expression vectors may be viral vectors or non-viral vectors. The term “nonviral vector” refers to an autonomously replicating, extrachromosomal circular DNA molecule, distinct from the normal genome and nonessential for cell survival under nonselective conditions capable of effecting the expression of a coding sequence in the target cell. Plasmids are examples of non-viral vectors. In order to facilitate transfection of the target cells, the target cell may be exposed directly with the non-viral vector may under conditions that facilitate uptake of the non-viral vector. Examples of conditions which facilitate uptake of foreign nucleic acid by mammalian cells are well known in the art and include but are not limited to chemical means (such as Lipofectamine®, Thermo-Fisher Scientific), high salt, magnetic fields (electroporation).

In one embodiment, a non-viral vector may be provided in a non-viral delivery system. Non-viral delivery systems are typically complexes to facilitate transduction of the target cell with a nucleic acid cargo wherein the nucleic acid is complexed with agents such as cationic lipids (DOTAP, DOTMA), surfactants, biologicals (gelatin, chitosan), metals (gold, magnetic iron) and synthetic polymers (PLG, PEI, PAMAM). Numerous embodiments of non-viral delivery systems are known in the art including lipidic vector systems (Lee et al. (1997) Crit Rev Ther Drug Carrier Syst. 14: 173-206); polymer coated liposomes (U.S. Pat. No. 5,213,804; U.S. Pat. No. 5,013,556); cationic liposomes (U.S. Pat. No. 5,283,185; U.S. Pat. No. 5,578,475; U.S. Pat. No. 5,279,833; U.S. Pat. No. 5,334,761).

In another embodiment, the expression vector may be a viral vector. As used herein, the term viral vector is used in its conventional sense to refer to any of the obligate intracellular parasites having no protein-synthesizing or energy-generating mechanism and generally refers to any of the enveloped or non-enveloped animal viruses commonly employed to deliver exogenous transgenes to mammalian cells. A viral vector may be replication competent (e.g., substantially wild-type), conditionally replicating (recombinantly engineered to replicate under certain conditions) or replication deficient (substantially incapable of replication in the absence of a cell line capable of complementing the deleted functions of the virus). The viral vector can possess certain modifications to make it “selectively replicating,” i.e. that it replicates preferentially in certain cell types or phenotypic cell states, e.g., cancerous. Viral vector systems useful in the practice of the instant invention include, for example, naturally occurring or recombinant viral vector systems. Examples of viruses useful in the practice of the present invention include recombinantly modified enveloped or non-enveloped DNA and RNA viruses. For example, viral vectors can be derived from the genome of human or bovine adenoviruses, vaccinia virus, lentivirus, herpes virus, adeno-associated virus, lentivirus (e.g., human immunodeficiency virus) sindbis virus, and retroviruses (including but not limited to Rous sarcoma virus), and hepatitis B virus. Typically, genes of interest are inserted into such vectors to allow packaging of the gene construct, typically with accompanying viral genomic sequences, followed by infection of a sensitive host cell resulting in expression of the gene of interest.

To facilitate combination therapy with additional polypeptide therapeutic agents, the expression vector may encode one or more polypeptides in addition to the IL-10 variant polypeptide. When expressing multiple polypeptides as in the practice of the present invention, each polypeptide may be operably linked to an expression control sequence (monocistronic) or multiple polypeptides may be encoded by a polycistronic construct where multiple polypeptides are expressed under the control of a single expression control sequence. In one embodiment, the expression vector encoding the targeting antigen may optionally further encode one or more immunological modulators. Examples of immunological modulators useful in the practice of the present invention include but are not limited to cytokines. The expressed cytokines can be directed for intracellular expression or expressed with a signal sequence for extracellular presentation or secretion.

The expression vector may optionally provide an expression cassette comprising a nucleic acid sequence encoding a “rescue” gene (e.g., a nucleic acid sequence, the expression of which renders the cell susceptible to killing by external factors or causes a toxic condition in the cell such that the cell is killed). Providing a rescue gene enables selective cell killing of transduced cells. Thus the rescue gene provides an additional safety precaution when said constructs are incorporated into the cells of a mammalian subject to prevent undesirable spreading of transduced cells or the effects of replication competent vector systems. In one embodiment, the rescue gene is the thymidine kinase (TK) gene (see e.g., U.S. Pat. No. 5,631,236 and U.S. Pat. No. 5,601,818) in which the cells expressing the TK gene product are susceptible to selective killing by the administration of ganciclovir.

### Methods of the Disclosure

Administration of any one of the therapeutic compositions described herein, e.g., recombinant polypeptides, IL-10 polypeptide variants, nucleic acids, recombinant cells, and pharmaceutical compositions, can be used to treat patients in the treatment of relevant health conditions and diseases, such as cancers, autoimmune diseases, and chronic infections. In some embodiments, recombinant polypeptides, IL-10 polypeptide variants, nucleic acids, recombinant cells, and pharmaceutical compositions as described herein can be incorporated into therapeutic agents for use in methods of treating an individual who has, who is suspected of having, or who may be at high risk for developing one or more autoimmune disease or conditions associated with IL-10 signaling. Exemplary autoimmune disease or conditions can include, without limitation, cancers, immune diseases, and chronic infection. In some embodiments, the individual is a patient under the care of a physician.

Accordingly, in one aspect, some embodiments of the disclosure relate to methods for modulating IL-10-mediated signaling in a subject, wherein the methods include administering to the subject a composition including one or more of: (a) a recombinant polypeptide of the disclosure; (b) a recombinant nucleic acid of the disclosure; (c) a recombinant cell; and (d) a pharmaceutically composition of the disclosure. In some embodiments, the methods include administering a therapeutically effective amount of the recombinant polypeptide of the disclosure.

In another aspect, some embodiments of the disclosure relate to methods for the treatment of a health condition in a subject in need thereof, wherein the methods includes administering to the subject a composition including one or more of: (a) a recombinant polypeptide of the disclosure; (b) a recombinant nucleic acid of the disclosure; (c) a recombinant cell; and (d) a pharmaceutically composition of the disclosure. In some embodiments, the methods include administering a therapeutically effective amount of the recombinant polypeptide of the disclosure.

In some embodiments, the disclosed pharmaceutical composition is formulated to be compatible with its intended route of administration. The recombinant polypeptides of the disclosure may be given orally or by inhalation, but it is more likely that they will be administered through a parenteral route. Examples of parenteral routes of administration include, for example, intravenous, intradermal, subcutaneous, transdermal (topical), transmucosal, and rectal administration. Solutions or suspensions used for parenteral application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates and agents for the adjustment of tonicity such as sodium chloride or dextrose. pH can be adjusted with acids or bases, such as mono- and/or di-basic sodium phosphate, hydrochloric acid or sodium hydroxide (e.g., to a pH of about 7.2-7.8, e.g., 7.5). The parenteral preparation can be enclosed in ampoules, disposable syringes or multiple dose vials made of glass or plastic.

Dosage, toxicity and therapeutic efficacy of such subject recombinant polypeptides of the disclosure can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit high therapeutic indices are generally suitable. While compounds that exhibit toxic side effects may be used, care should be taken to design a delivery system that targets such compounds to the site of affected tissue in order to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED₅₀ with little or no toxicity. The dosage may vary within this range depending upon the dosage form employed and the route of administration utilized. For any compound used in the method of the disclosure, the therapeutically effective dose can be estimated initially from cell culture assays. A dose may be formulated in animal models to achieve a circulating plasma concentration range that includes the IC₅₀ (e.g., the concentration of the test compound which achieves a half-maximal inhibition of symptoms) as determined in cell culture. Such information can be used to more accurately determine useful doses in humans. Levels in plasma may be measured, for example, by high performance liquid chromatography.

The therapeutically effective amount of a subject recombinant polypeptide of the disclosure (e.g, an effective dosage) depends on the polypeptide selected. For instance, single dose amounts in the range of approximately 0.001 to 0.1 mg/kg of patient body weight can be administered; in some embodiments, about 0.005, 0.01, 0.05 mg/kg may be administered. In some embodiments, 600,000 IU/kg is administered (IU can be determined by a lymphocyte proliferation bioassay and is expressed in International Units (IU). The compositions can be administered one from one or more times per day to one or more times per week; including once every other day. The skilled artisan will appreciate that certain factors may influence the dosage and timing required to effectively treat a subject, including but not limited to the severity of the disease, previous treatments, the general health and/or age of the subject, and other diseases present. Moreover, treatment of a subject with a therapeutically effective amount of the subject recombinant polypeptides of the disclosure can include a single treatment or, can include a series of treatments. In some embodiments, the compositions are administered every 8 hours for five days, followed by a rest period of 2 to 14 days, e.g., 9 days, followed by an additional five days of administration every 8 hours.

Non-limiting exemplary embodiments of the disclosed methods for modulating IL-10-mediated signaling in a subject and/or for the treatment of a health condition in a subject in need thereof can include one or more of the following features. In some embodiments, the administered composition confers a cell-type biased IL-10 signaling in the subject compared to a composition comprising a reference IL-10 polypeptide lacking the amino acid substitution(s). As described in greater detail in the Examples, an exemplary IL-10 polypeptide variant of the disclosure, 10-DE, elicits cell-type biased signaling responses in vivo, acting as a full STAT3 agonist in monocytes and macrophages, but a weak STAT3 agonist in CD4+, CD8+ T cells, NK cells, and B cells. In particular, IL-10 variant 10-DE confers a reduction in a STAT3-mediated pro-inflammatory function in bulk PBMCs, such as B cells, CD4+ T cells, CD8+ T cells, and/or NK cells while substantially retaining its STAT3-anti-inflammatory mediated function in monocytes and macrophages, thereby uncoupling the anti-inflammatory and immunostimulatory functions of IL-10 in vivo. Accordingly, in some embodiments, the amino acid substitutions in the recombinant IL-10 polypeptide variants disclosed herein result in a cell-type biased IL-10 signaling which involves a reduction of IL-10 signaling in bulk PBMCs, B cells, CD4+ T cells, CD8+ T cells, and/or NK cells while substantially retains IL-10 signaling in monocytes and macrophages.

Accordingly, in some embodiments, the administered composition results in a cell-type biased IL-10 signaling which involves a reduction of IL-10 signaling in bulk PBMCs, B cells, T cells, and/or NK cells while substantially retains IL-10 signaling in monocytes. In some embodiments, the cell-type biased IL-10 signaling includes a reduction of IL-10 signaling in bulk PBMCs, B cells, T cells, and/or NK cells while substantially retains IL-10 signaling in macrophages. In some embodiments, the administered composition suppresses inflammatory cytokine production in bulk PBMCs in the subject. In some embodiments, the administered composition downregulates MHC-II on activated monocytes. In some embodiments, the administered composition suppresses inflammatory cytokine production in human monocyte-derived macrophages. In some embodiments, the administered composition does not potentiate activity of activated CD8+ T cells. In some embodiments, the administered composition does not potentiate IFN-γ and/or Granzyme B production by CD8+ T cells.

In some embodiments, the administered composition results in a biased IL-10 signaling as determined by, for example, phosphorylation of STAT3, compared to a composition comprising a reference IL-10 polypeptide lacking the amino acid substitution(s). In some embodiments, the biased IL-10 signaling includes a reduction in a STAT3 phosphorylation by at least 10%, e.g., by at least 10%, at least 20%, at least 30%, at least 40%, at least 50%, at least 60%, at least 70%, at least 80%, or at least 90% when compared to a reference IL-10 polypeptide lacking the amino acid substitution(s).

In some embodiments, the administered composition results in a reduction in one or more STAT3-mediated pro-inflammatory functions. Non-limiting examples of STAT3-mediated pro-inflammatory function include cytokine production, chemokine production, immune cell proliferation, and immune cell recruitment. In some embodiments, the STAT3-mediated pro-inflammatory function is reduced from about 20% to about 100%, as determined by a gene expression assay, a phospho-flow signaling assay, and/or an enzyme-linked immunosorbent assay (ELISA). In some embodiments, the administered composition results in a reduction in one or more STAT3-mediated pro-inflammatory functions, as determined by the polypeptides’ capacity to induce expression of a pro-inflammatory gene. Non-limiting examples of pro-inflammatory genes include IFN-γ, granzyme B, granzyme A, perforin, TNF-α, GM-CSF, and MIP1α.

In some embodiments, the STAT3-mediated pro-inflammatory function (e.g., as measured in Daudi, Jurkat or YT-1 cells) is reduced about 20% to about 100%, for example, about 20% to about 50%, about 30% to about 60%, about 40% to about 70%, about 50% to about 80%, about 40% to about 90%, bout 50% to about 100%, about 40% to about 80%, about 30% to about 70%, about 20% to about 80%, about 20% to about 70%, about 20% to about 60%, about 30% to about 80%, about 30% to about 90%, or about 30% to about 100% compared to a reference IL-10 lacking the amino acid substitution(s), in some embodiments without significantly altering STAT3-mediated function in primary monocytes.

In some embodiments, the administered composition substantially retain one or more STAT3-mediated function. Examples of STAT3-mediated functions include, but are not limited suppression of monocyte and macrophage activation, cytokine production, chemokine production, and MHC expression. In some embodiments, the administered composition substantially retains one or more STAT3-mediated function, as determined by the polypeptides’ capacity to suppress expression of a biomarker, such as IL-6, TNF-α, IL-1b, IL-8, IL-12, and HLA-DR.

In some embodiments, the administered composition confers a reduced capacity to induce expression of a pro-inflammatory gene selected from IFN-γ, granzyme B, granzyme A, perforin, TNF-α, GM-CSF, and MIP1α in the subject. In some embodiments, the administered composition substantially retains its capacity to reduce expression of a gene selected from IL-6, TNF-α, IL-Ib, IL-8, IL-12, and HLA-DR in the subject. In some embodiments, the administration of the pharmaceutical composition does not activate T-cell activity in the subject.

In some embodiments, the administered composition enhances antitumor immunity in a tumor microenvironment. In some embodiments, the administered composition enhances antitumor immunity in a tumor microenvironment epithelial protection and regeneration. In some embodiments, the condition is a cancer, an immune disease, an autoimmune disease, or a chronic infection.

In some embodiments, provided herein are methods for the treatment of a health condition in a subject in need thereof, wherein the health condition is a cancer. The term cancer generally refers to the presence of cells possessing characteristics typical of cancer-causing cells, such as uncontrolled proliferation, immortality, metastatic potential, rapid growth and proliferation rate, and certain characteristic morphological features. Cancer cells are often observed aggregated into a tumor, but such cells can exist alone within an animal subject, or can be a non-tumorigenic cancer cell, such as a leukemia cell. Thus, the terms “cancer” or can encompass reference to a solid tumor, a soft tissue tumor, or a metastatic lesion. As used herein, the term “cancer” includes premalignant, as well as malignant cancers. In some embodiments, the cancer is a solid tumor, a soft tissue tumor, or a metastatic lesion.

In some embodiments, provided herein are methods for the treatment of a cancer in a subject in need thereof, wherein the cancer selected from the group consisting of an acute myeloma leukemia, an anaplastic lymphoma, an astrocytoma, a B-cell cancer, a breast cancer, a colon cancer, an ependymoma, an esophageal cancer, a glioblastoma, a glioma, a leiomyosarcoma, a liposarcoma, a liver cancer, a lung cancer, a mantle cell lymphoma, a melanoma, a neuroblastoma, a non-small cell lung cancer, an oligodendroglioma, an ovarian cancer, a pancreatic cancer, a peripheral T-cell lymphoma, a renal cancer, a sarcoma, a stomach cancer, a carcinoma, a mesothelioma, and a sarcoma.

In some embodiments, the immune disease is an autoimmune disease. In some embodiments, the autoimmune disease is selected from the group consisting of rheumatoid arthritis, insulin-dependent diabetes mellitus, hemolytic anemias, rheumatic fever, thyroiditis, Crohn’s disease, myasthenia gravis, glomerulonephritis, autoimmune hepatitis, multiple sclerosis, alopecia areata, psoriasis, vitiligo, dystrophic epidermolysis bullosa, systemic lupus erythematosus, graft vs. host disease, ulcerative colitis, pancreatitis, psoriatic arthritis, and diabetic foot ulcer. In some embodiments, the subject is a mammal. In some embodiments, the mammal is a human. In some embodiments, the subject has or is suspected of having a condition associated with IL-10 mediated signaling.

In some embodiments, provided herein are methods for the treatment of a health condition in a subject in need thereof, wherein the health condition is a malignancy associated with chronic inflammatory conditions, such as a chronic infection by a microorganism, for example, by a bacterium, a microgfungus, or a virus. Accordingly, some embodiments of the disclosure relate to methods for treating a malignancy associated with a bacterial infection, such as sepsis. Some embodiments of the disclosure relate to methods for treating a malignancy associated with a chronic viral infection. Non-limiting examples of malignancies associated with a chronic viral infection include acute respiratory distress syndrome (ARDS) and cytokine release syndrome (CRS). In some embodiments, the viral infection is a respiratory infection. In some embodiments, the infection is caused by a virus belonging to a species of the Human orthopneumovirus genus, a species of the Enterovirus family, a species of the Coronaviridae family, or a subtype of the Orthomyxoviridae family. In some embodiments, the orthomyxovirus is an influenza A virus or a Parainfluenza virus. In some embodiments, the influenza A virus is selected from the group consisting of subtypes H1N1, H1N2, H2N2, H3N1, H3N2, H3N8, H5N1, H5N2, H5N3, H5N8, H5N9, H7N1, H7N2, H7N3, H7N4, H7N7, H7N9, H9N2, and H10N7. In some embodiments, the parainfluenza virus is selected from the group consisting of subtypes HPIV-1, HPIV-2, HPIV-3, and HPIV-4. In some embodiments, the infection is caused by a coronavirus. In some embodiments, the coronavirus is β-CoV severe acute respiratory syndrome coronavirus (SARS-CoV), severe acute respiratory syndrome coronavirus 2 (SARS-CoV-2). In some embodiments, the coronavirus β-CoV infection is associated with one or more subgenus Sarbecovirus selected from the group consisting of severe acute respiratory syndrome coronavirus SARSr-CoV (which includes all its strains such as SARS-CoV, SARS-CoV-2, and Bat SL-CoV-WIV1), subgenus Merbecovirus consisting of Tylonycteris bat coronavirus HKU4 (BtCoV-HKU4), Pipistrellus bat coronavirus HKU5 (BtCoV-HKU5), and Middle East respiratory syndrome-related coronavirus MERS-CoV (which includes the species HCoV-229E, HCoV-NL63, HCoV-OC43, HCoV-HKU1). In some embodiments, the human orthomyxovirus is a human respiratory syncytial virus (HRSV). In some embodiments, the HRSV is associated with subtype A and/or subtype B.

### Additional Therapies

As discussed supra, any one of the recombinant polypeptides, nucleic acids, recombinant cells, cell cultures, and/or pharmaceutical compositions described herein can be administered in combination with one or more additional therapeutic agents such as, for example, chemotherapeutics, anti-cancer agents, immunosuppressive agents, immunosuppressants, or anti-inflammatory agents. Administration “in combination with” one or more additional therapeutic agents includes simultaneous (concurrent) and consecutive administration in any order. In some embodiments, the one or more additional therapeutic agents, chemotherapeutics, anti-cancer agents, immunosuppressants, or anti-inflammatory agents is selected from the group consisting of chemotherapy, radiotherapy, immunotherapy, hormonal therapy, toxin therapy, surgery, and disease modifying anti-rheumatic drugs (DMARDs). Various classes of anti-inflammatory agents can be used. Non-limiting examples include cytokine antagonists, cytokine receptor antagonists, soluble receptors, integrin antagonists, corticosteroids, kinase inhibitors, S1P agonists, and PDE4 antagonists.

In some embodiments, the methods of treatment as described herein further include administration of a composition that suppresses inflammatory immune responses, e.g., anti-inflammatory drugs. Suitable anti-inflammatory drugs include, but are not limited to, TNF antagonists (e.g., adalimumab, infliximab), IL-23 antagonists (e.g., ustekinumab), IL-17 antagonists (e.g. secukinumab), IL-1 antagonists (e.g. anakinra), IL-6 antagonists (e.g. tocilizumab), IL-12 antagonists (e.g., ustekinumab), IL-2 antagonists (e.g., Daclizumab), integrin antagonists (e.g., vedolizumab), corticosteroids (e.g. prednisone), aminosalicylates (e.g. mesalamine, balsaazide, olsalazine), methotrexate, azathioprine, leflunomide, chloroquine, calcineurin inhibitors (cyclosporine, tacrolimus), abatacept, rituximab, JAK inhibitors (e.g., tofacitinib), PDE4 inhibitors (e.g., apremilast), mTOR inhibitors (e.g., sirolimus), S1P receptor agonists (e.g., Ozanimod).

In some embodiments, the methods of treatment as described herein further include administration of a composition comprising a tissue regenerative factor such as a Wnt agonist or a Notch agonist.

“Chemotherapy” and “anti-cancer agent” are used interchangeably herein. Various classes of anti-cancer agents can be used. Non-limiting examples include: alkylating agents, antimetabolites, anthracyclines, plant alkaloids, topoisomerase inhibitors, podophyllotoxin, antibodies (e.g., monoclonal or polyclonal), tyrosine kinase inhibitors (e.g., imatinib mesylate (Gleevec® or Glivec®)), hormone treatments, soluble receptors and other antineoplastics.

Topoisomerase inhibitors are also another class of anti-cancer agents that can be used herein. Topoisomerases are essential enzymes that maintain the topology of DNA. Inhibition of type I or type II topoisomerases interferes with both transcription and replication of DNA by upsetting proper DNA supercoiling. Some type I topoisomerase inhibitors include camptothecins: irinotecan and topotecan. Examples of type II inhibitors include amsacrine, etoposide, etoposide phosphate, and teniposide. These are semisynthetic derivatives of epipodophyllotoxins, alkaloids naturally occurring in the root of American Mayapple

### Podophyllum Peltatum

Antineoplastics include the immunosuppressant dactinomycin, doxorubicin, epirubicin, bleomycin, mechlorethamine, cyclophosphamide, chlorambucil, ifosfamide. The antineoplastic compounds generally work by chemically modifying a cell’s DNA.

Alkylating agents can alkylate many nucleophilic functional groups under conditions present in cells. Cisplatin and carboplatin, and oxaliplatin are alkylating agents. They impair cell function by forming covalent bonds with the amino, carboxyl, sulfhydryl, and phosphate groups in biologically important molecules.

Vinca alkaloids bind to specific sites on tubulin, inhibiting the assembly of tubulin into microtubules (M phase of the cell cycle). The vinca alkaloids include: vincristine, vinblastine, vinorelbine, and vindesine.

In some embodiments, the methods of treatment as described herein further include administration of a compound that inhibits one or more immune checkpoint molecules. In some embodiments, the one or more immune checkpoint molecules include one or more of CTLA4, PD-1, PD-L1, A2AR, B7-H3, B7-H4, TIM3, and combinations of any thereof. In some embodiments, the compound that inhibits the one or more immune checkpoint molecules includes an antagonistic antibody. In some embodiments, the antagonistic antibody is ipilimumab, nivolumab, pembrolizumab, durvalumab, atezolizumab, tremelimumab, or avelumab.

Anti-metabolites resemble purines (azathioprine, mercaptopurine) or pyrimidine and prevent these substances from becoming incorporated in to DNA during the “S” phase of the cell cycle, stopping normal development and division. Anti-metabolites also affect RNA synthesis.

Plant alkaloids and terpenoids are obtained from plants and block cell division by preventing microtubule function. Since microtubules are vital for cell division, without them, cell division cannot occur. The main examples are vinca alkaloids and taxanes. Podophyllotoxin is a plant-derived compound which has been reported to help with digestion as well as used to produce two other cytostatic drugs, etoposide and teniposide. They prevent the cell from entering the G1 phase (the start of DNA replication) and the replication of DNA (the S phase).

Taxanes as a group includes paclitaxel and docetaxel. Paclitaxel is a natural product, originally known as Taxol and first derived from the bark of the Pacific Yew tree. Docetaxel is a semi-synthetic analogue of paclitaxel. Taxanes enhance stability of microtubules, preventing the separation of chromosomes during anaphase.

In some embodiments, the anti-cancer agents can be selected from remicade, docetaxel, celecoxib, melphalan, dexamethasone (Decadron®), steroids, gemcitabine, cisplatinum, temozolomide, etoposide, cyclophosphamide, temodar, carboplatin, procarbazine, gliadel, tamoxifen, topotecan, methotrexate, gefitinib (Iressa®), taxol, taxotere, fluorouracil, leucovorin, irinotecan, xeloda, CPT-11, interferon alpha, pegylated interferon alpha (e.g., PEG INTRON-A), capecitabine, cisplatin, thiotepa, fludarabine, carboplatin, liposomal daunorubicin, cytarabine, doxetaxol, pacilitaxel, vinblastine, IL-2, GM-CSF, dacarbazine, vinorelbine, zoledronic acid, palmitronate, biaxin, busulphan, prednisone, bortezomib (Velcade®), bisphosphonate, arsenic trioxide, vincristine, doxorubicin (Doxil®), paclitaxel, ganciclovir, adriamycin, estrainustine sodium phosphate (Emcyt®), sulindac, etoposide, and combinations of any thereof.

In other embodiments, the anti-cancer agent can be selected from bortezomib, cyclophosphamide, dexamethasone, doxorubicin, interferon-alpha, lenalidomide, melphalan, pegylated interferon-alpha, prednisone, thalidomide, or vincristine.

In some embodiments, the methods of treatment as described herein further include an immunotherapy. In some embodiments, the immunotherapy includes administration of one or more checkpoint inhibitors. Accordingly, some embodiments of the methods of treatment described herein include further administration of a compound that inhibits one or more immune checkpoint molecules. In some embodiments, the compound that inhibits the one or more immune checkpoint molecules includes an antagonistic antibody. In some embodiments, the antagonistic antibody is ipilimumab, nivolumab, pembrolizumab, durvalumab, atezolizumab, tremelimumab, or avelumab.

In some aspects, the one or more anti-cancer therapies include radiation therapy. In some embodiments, the radiation therapy can include the administration of radiation to kill cancerous cells. Radiation interacts with molecules in the cell such as DNA to induce cell death. Radiation can also damage the cellular and nuclear membranes and other organelles. Depending on the radiation type, the mechanism of DNA damage may vary as does the relative biologic effectiveness. For example, heavy particles (i.e. protons, neutrons) damage DNA directly and have a greater relative biologic effectiveness. Electromagnetic radiation results in indirect ionization acting through short-lived, hydroxyl free radicals produced primarily by the ionization of cellular water. Clinical applications of radiation consist of external beam radiation (from an outside source) and brachytherapy (using a source of radiation implanted or inserted into the patient). External beam radiation consists of X-rays and/or gamma rays, while brachytherapy employs radioactive nuclei that decay and emit alpha particles, or beta particles along with a gamma ray. Radiation also contemplated herein includes, for example, the directed delivery of radioisotopes to cancer cells. Other forms of DNA damaging factors are also contemplated herein such as microwaves and UV irradiation.

Radiation may be given in a single dose or in a series of small doses in a dose-fractionated schedule. The amount of radiation contemplated herein ranges from about 1 to about 100 Gy, including, for example, about 5 to about 80, about 10 to about 50 Gy, or about 10 Gy. The total dose may be applied in a fractioned regime. For example, the regime may include fractionated individual doses of 2 Gy. Dosage ranges for radioisotopes vary widely and depends on the half-life of the isotope and the strength and type of radiation emitted. When the radiation includes use of radioactive isotopes, the isotope may be conjugated to a targeting agent, such as a therapeutic antibody, which carries the radionucleotide to the target tissue (e.g., tumor tissue).

Surgery described herein includes resection in which all or part of a cancerous tissue is physically removed, exercised, and/or destroyed. Tumor resection refers to physical removal of at least part of a tumor. In addition to tumor resection, treatment by surgery includes laser surgery, cryosurgery, electrosurgery, and microscopically controlled surgery (Mohs surgery). Removal of precancers or normal tissues is also contemplated herein.

Accordingly, in some embodiments of the methods disclosed herein, the composition is administered to the subject individually as a first therapy or in combination with a second therapy. In some embodiments, the second therapy is selected from the group consisting of chemotherapy, radiotherapy, DMARDs, immunotherapy, hormonal therapy, toxin therapy, and surgery. In some embodiments, the first therapy and the second therapy are administered concomitantly. In some embodiments, the first therapy is administered at the same time as the second therapy. In some embodiments, the first therapy and the second therapy are administered sequentially. In some embodiments, the first therapy is administered before the second therapy. In some embodiments, the first therapy is administered after the second therapy. In some embodiments, the first therapy is administered before and/or after the second therapy. In some embodiments, the first therapy and the second therapy are administered in rotation. In some embodiments, the first therapy and the second therapy are administered together in a single formulation.

### Kits

Also provided herein are kits including the IL-10 partial agonists, recombinant nucleic acids, recombinant cells, or pharmaceutical compositions provided and described herein as well as written instructions for making and using the same. For example, provided herein, in some embodiments, are kits that include one or more of: a recombinant polypeptide of the disclosure, an IL-10 partial agonist of the disclosure, a recombinant nucleic acids of the disclosure, a recombinant cell of the disclosure, or a pharmaceutical composition of the disclosure; and instructions for use thereof. In some embodiments, the kits of the disclosure further include one or more syringes (including pre-filled syringes) and/or catheters (including pre-filled syringes) used to administer one any of the provided recombinant nucleic acids, recombinant cells, or pharmaceutical compositions to an individual; and instructions for use thereof. In some embodiments, a kit can have one or more additional therapeutic agents that can be administered simultaneously or sequentially with the other kit components for a desired purpose, e.g., for modulating an activity of a cell, inhibiting a target cancer cell, or treating a disease in an individual in need thereof.

Any of the above-described kits can further include one or more additional reagents, where such additional reagents can be selected from: dilution buffers; reconstitution solutions, wash buffers, control reagents, control expression vectors, negative control polypeptides, positive control polypeptides, reagents for in vitro production of the recombinant polypeptides.

In some embodiments, the components of a kit can be in separate containers. In some other embodiments, the components of a kit can be combined in a single container. For example, in some embodiments of the disclosure, the kit includes one or more of the recombinant IL-10 polypeptides, recombinant nucleic acids, recombinant cells, or pharmaceutical compositions as described herein in one container (e.g., in a sterile glass or plastic vial) and a further therapeutic agent in another container (e.g., in a sterile glass or plastic vial).

In some embodiments, a kit can further include instructions for using the components of the kit to practice a method described herein. For example, the kit can include a package insert including information concerning the pharmaceutical compositions and dosage forms in the kit. Generally, such information aids patients and physicians in using the enclosed pharmaceutical compositions and dosage forms effectively and safely. For example, the following information regarding a combination of the disclosure may be supplied in the insert: pharmacokinetics, pharmacodynamics, clinical studies, efficacy parameters, indications and usage, contraindications, warnings, precautions, adverse reactions, overdosage, proper dosage and administration, how supplied, proper storage conditions, references, manufacturer/distributor information and intellectual property information.

In some embodiments, a kit can further include instructions for using the components of the kit to practice the methods. The instructions for practicing the methods are generally recorded on a suitable recording medium. For example, the instructions can be printed on a substrate, such as paper or plastic, etc. The instructions can be present in the kit as a package insert, in the labeling of the container of the kit or components thereof (e.g., associated with the packaging or sub-packaging), etc. The instructions can be present as an electronic storage data file present on a suitable computer readable storage medium, e.g. CD-ROM, diskette, flash drive, etc. In some instances, the actual instructions are not present in the kit, but means for obtaining the instructions from a remote source (e.g., via the internet), can be provided. An example of this embodiment is a kit that includes a web address where the instructions can be viewed and/or from which the instructions can be downloaded. As with the instructions, this means for obtaining the instructions can be recorded on a suitable substrate.

All publications and patent applications mentioned in this disclosure are herein incorporated by reference to the same extent as if each individual publication or patent application was specifically and individually indicated to be incorporated by reference.

No admission is made that any reference cited herein constitutes prior art. The discussion of the references states what their authors assert, and the Applicant reserves the right to challenge the accuracy and pertinence of the cited documents. It will be clearly understood that, although a number of information sources, including scientific journal articles, patent documents, and textbooks, are referred to herein; this reference does not constitute an admission that any of these documents forms part of the common general knowledge in the art.

The discussion of the general methods given herein is intended for illustrative purposes only. Other alternative methods and alternatives will be apparent to those of skill in the art upon review of this disclosure, and are to be included within the spirit and purview of this application.

### EXAMPLES

The practice of the present invention will employ, unless otherwise indicated, conventional techniques of molecular biology, microbiology, cell biology, biochemistry, nucleic acid chemistry, and immunology, which are well known to those skilled in the art. Such techniques are explained fully in the literature, such as Sambrook, J., & Russell, D. W. (2012). Molecular Cloning: A Laboratory Manual (4th ed.). Cold Spring Harbor, NY: Cold Spring Harbor Laboratory and Sambrook, J., & Russel, D. W. (2001). Molecular Cloning: A Laboratory Manual (3rd ed.). Cold Spring Harbor, NY: Cold Spring Harbor Laboratory (jointly referred to herein as “Sambrook”); Ausubel, F. M. (1987). Current Protocols in Molecular Biology. New York, NY: Wiley (including supplements through 2014); Bollag, D. M. et al. (1996). Protein Methods. New York, NY: Wiley-Liss; Huang, L. et al. (2005). Nonviral Vectors for Gene Therapy. San Diego: Academic Press; Kaplitt, M. G. et al. (1995). Viral Vectors: Gene Therapy and Neuroscience Applications. San Diego, CA: Academic Press; Lefkovits, I. (1997). The Immunology Methods Manual: The Comprehensive Sourcebook of Techniques. San Diego, CA: Academic Press; Doyle, A. et al. (1998). Cell and Tissue Culture: Laboratory Procedures in Biotechnology. New York, NY: Wiley; Mullis, K. B., Ferré, F. & Gibbs, R. (1994). PCR: The Polymerase Chain Reaction. Boston: Birkhauser Publisher; Greenfield, E. A. (2014). Antibodies: A Laboratory Manual (2nd ed.). New York, NY: Cold Spring Harbor Laboratory Press; Beaucage, S. L. et al. (2000). Current Protocols in Nucleic Acid Chemistry. New York, NY: Wiley, (including supplements through 2014); and Makrides, S. C. (2003). Gene Transfer and Expression in Mammalian Cells. Amsterdam, NL: Elsevier Sciences B.V., the disclosures of which are incorporated herein by reference.

Additional embodiments are disclosed in further detail in the following examples, which are provided by way of illustration and are not in any way intended to limit the scope of this disclosure or the claims.

### Example 1

### General Experimental Procedures

### Protein Production and Purification

For yeast-binding studies, and SPR, the ECDs of human IL-10Rα (2-235) and IL-10Rβ (20-220) were cloned into the pAcGP67a baculoviral vector with an N-terminal GP64 signal peptide, C-terminal 3C cleavage site followed by a biotin-acceptor peptide tag (BAP tag, GLNDIFEAQKIEW, SEQ ID NO: 31) and 6xHis tag. The baculovirus stocks were prepared by cotransfection of the BestBac™ DNA (Expression Systems) and the pAcGP67-A DNA into Spodoptera frugiperda (Sf9). Next, the viruses were used to infect the Trichoplusia ni (Hi5) cells. The proteins were purified from the supernatant of baculovirus infected Hi5 cells 72 hours after infection and purified with Ni-NTA resin (Qiagen) followed by size-exclusion chromatography (SEC) on a Superdex 200 column (GE). The proteins were maintained in HEPES buffered saline (HBS, 20 mM HEPES pH 7.4, 150 mM sodium chloride). IL-10Rβ ECD was site-specifically biotinylated at the C-terminal BAP tag using BirA ligase and re-purified by size exclusion chromatography.

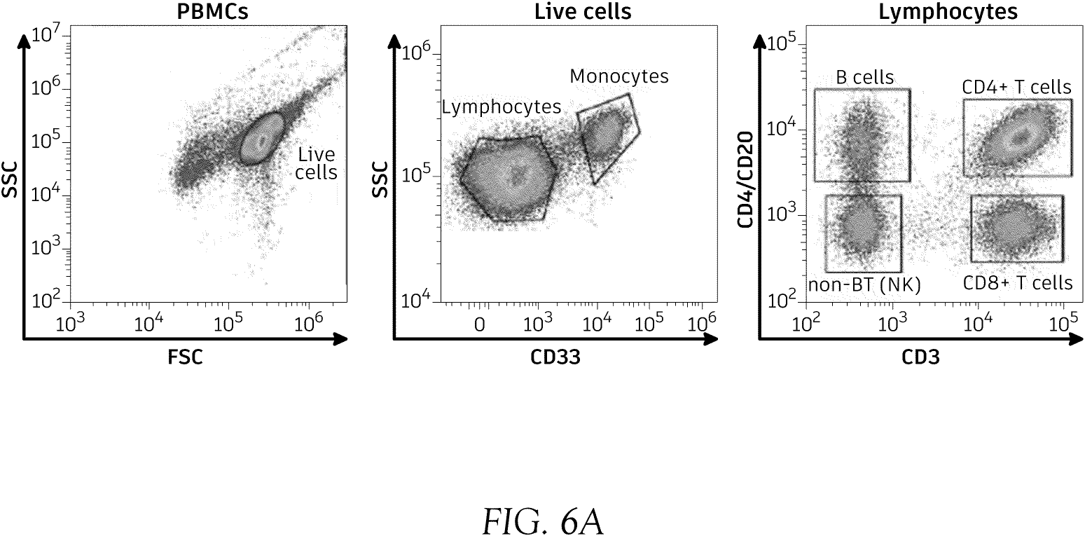

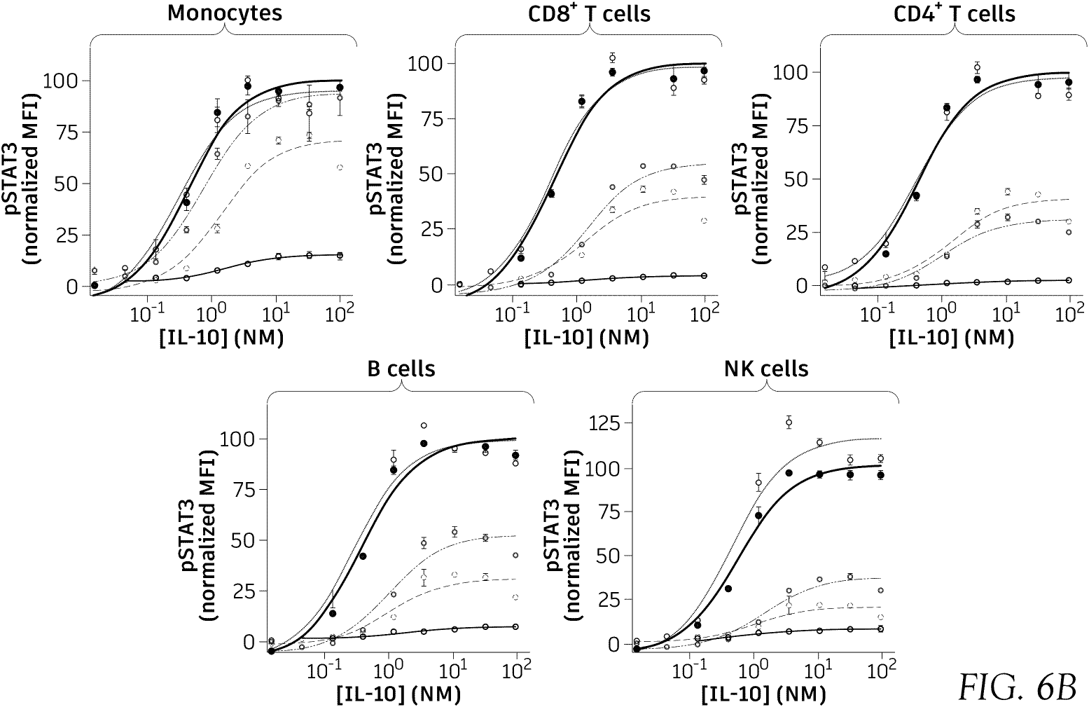

For cryo-EM structural studies, a glyco-mutant version the IL-10Rβ ECDs (FIG. 6A), was cloned into the pAcGP67a baculoviral vector with an N-terminal GP64 signal peptide and C-terminal 6xHis tag, expressed and purified as described above, followed by size-exclusion chromatography (SEC) on a Superdex 200 column (GE). Affinity matured IL-10 (“Super-10”, clone 5.1) and the IL-10Rα ECD were cloned into the pD649 mammalian expression vector containing an N-terminal HA signal peptide and C-terminal 6xHis-tag. DNA was transiently transfected into Expi-293F cells (Thermo Fisher Scientific) using Expifectamine transfection reagent (Thermo Fisher Scientific). Approximately 72-96 hours after transfection, cell supernatant was harvested and proteins were purified with Ni-NTA resin (Qiagen) followed by size-exclusion chromatography (SEC) on a Superdex 200 column (GE) in HEPES buffered saline (HBS, 30 mM HEPES pH 7.4, 150 mM sodium chloride). Following SEC, the individual proteins were incubated overnight at a 1:1:1.2 molar ratio of IL-10: IL-10Rα:IL-10Rβ and re-purified by SEC on an S200 column (GE).

For signaling and functional experiments, IL-10 variants were cloned into the pD649 mammalian expression vector containing an N-terminal HA signal peptide and C-terminal 6xHis-tag. DNA was transiently transfected into Expi-293F cells (Thermo Fisher Scientific) using Expifectamine transfection reagent (Thermo Fisher Scientific). Approximately 72-96 hours after transfection, cell supernatant was harvested and proteins were purified with Ni-NTA resin (Qiagen) followed by size-exclusion chromatography (SEC) on a Superdex 200 column (GE) in HEPES buffered saline (HBS, 30 mM HEPES pH 7.4, 150 mM sodium chloride).

### Surface Plasmon Resonance

Dissociation constants (K_{D}) for IL-10Rβ-binding by affinity matured IL-10 clone 5.1, either alone or pre-bound with soluble IL-10Rα, was determined by surface plasmon resonance (SPR) using the BIAcore T100 instrument (GE Healthcare). First, biotinylated IL-10Rβ was captured on a streptavidin-coated (SA) sensor chip (GE Healthcare) with immobilization density in the range of 100 resonance units (RU). Similarly, a control flow cell was also prepared with an off-target protein (EpoR) for reference subtraction. The binding kinetics were performed at 25° C. with a flow rate of 50 µl/min. IL-10 or the IL-10/IL-10Rα complex were serially diluted in HBS buffer supplemented with 0.005% P20 surfactant (GE Healthcare) and injected over the SA chip. Dissociation kinetics were monitored for 100-500 seconds, as needed, based on the ligand affinity. The steady state affinity was determined using the BIAcore T100 evaluation software. The kinetic binding curves were generated by plotting the time-dependent response units in Prism 8 (GraphPad).

### Cell Culture

THP-1 (ATCC TIB-202), YT-1 (RRID: CVCL EJ05), Jurkat (ATCC TIB-152), and Daudi (ATCC CCL-213) cells were all grown in RPMI (Dulbecco’s Modified Eagle Medium) supplemented with 10% v/v fetal bovine serum, penicillin-streptomycin, and 2 mM GlutaMAX™ (Gibco). The cells were maintained at 37° C. with 5% CO2. Expi293F cells were grown in serum free Expi293 expression media (Thermo Fisher Scientific) and maintained at 37° C. with 5% CO₂.

### Phospho-Flow Signaling Assays in Cell Lines

THP-1, YT-1, Jurkat, and Daudi cells were plated in 96-well plates and stimulated with WT or mutant IL-10 for 20 min at 37° C., followed by fixation with paraformaldehyde (Electron Microscopy Sciences) for 10 minutes at room temperature. The cells were permeabilized for intracellular staining by treatment with ice cold methanol (Fisher) for 30 min at -20° C. The cells were then incubated with Alexa Fluor 647 conjugated Anti-Stat3 (pY705) antibody (BD Biosciences) at a 1:50 dilution for 1 hour at room temperature in autoMACS buffer (Miltenyi). The background fluorescence of the unstimulated samples was subtracted from all samples. Data was acquired using CytoFlex, flow cytometer instrument (Beckman Coulter). The MFI values were normalized to the maximal WT IL-10 value within each experiment and plotted in Prism 8 (GraphPad). The dose-response curves were generated using the “sigmoidal dose-response” analysis.

Receptor surface expression was analyzed by resuspending live cells in autoMACS buffer and incubating with Human TruStain FcX (Biolegend) followed by anti-human IL-10Rα AlexaFluor 647 (BD Biosciences) and anti-human IL-10Rβ AlexaFluor 488 (R&D) antibodies for 30 minutes at 4° C. Fluorescent intensities were measured by flow cytometry on an Accuri C6 cytometer (BD Biosciences), and data were analyzed using FlowJo software (BD Biosciences).

### Phospho-Flow Signaling Assays in Human PBMCs

PBMCs were thawed in warm media, washed twice and resuspended at 0.5×10⁶ viable cells/mL. 200 uL of cells were plated per well in 96-well deep-well plates. After resting for 2 hours at 37° C., cells were stimulated with varying concentrations of WT or mutant IL-10 and incubated at 37° C. for 20 minutes. The PBMCs were then fixed with paraformaldehyde (Electron Microscopy Sciences) for 10 minutes at room temperature, and permeabilized for intracellular staining by treatment with ice cold methanol (Fisher) for 30 min at -20° C. The cells were washed with autoMACS buffer (Miltenyi), incubated with human TruStain FcX (Biolegend), and then stained with the following antibodies: CD3 Pacific Blue (BD), CD4 PerCP-Cy5.5 (BD), CD20 PerCp-Cy5.5 (BD), CD33 PE-Cy7 (BD), pSTAT-3 (Y705) AlexaFluor 647 (BD), and IL-10Rβ AlexaFluor 488 (R&D). The samples were then washed and resuspended in autoMACS buffer. Data was acquired using CytoFlex flow cytometer instrument (Beckman Coulter) and analyzed using FlowJo software (BD Biosciences). The MFI values were normalized to the maximal WT IL-10 value within each cell type and plotted in Prism 8 (GraphPad). The dose-response curves were generated using the “sigmoidal dose-response” analysis. Cells were gated on live cells based on forward versus side scatter profiles, followed by cell subset-specific gating as described previously.

### LPS Stimulation of Human PBMCs, Monocytes, and Macrophages

For stimulation of bulk PBMCs, PBMC were thawed in warm media, washed twice and resuspended in complete RPMI at 1×10⁷ cells/ml. Cells were plated at 500 µl/well in a 24 well culture dish and rested for 2 hours at 37° C. Cells were then incubated with 1 nM LPS (Sigma) alone or with 10 nM IL-10 (WT or mutant) for 24 hours. For cytokine measurements, cell supernatants were isolated and levels of TNF-α (Thermo Fisher Scientific), IL-6 (R&D), and CXCL8 (R&D) were measured by ELISA as per the manufacturer’s instructions.

For monocyte HLA-DR expression, cells were washed and resuspended in autoMACS buffer, incubated with human TruStain FcX (Biolegend) and stained with CD14 PE-Cy7 (BD) and anti-HLA-DR BV605 antibodies for 30 minutes at 4° C. Fluorescent intensities were measured using CytoFlex flow cytometer instrument (Beckman Coulter) and analyzed using FlowJo software (BD Biosciences). Cells were gated on live cells based on forward versus side scatter profiles, followed by monocytes specific gating (CD14⁺).

For analysis of primary human macrophages, PBMCs were thawed in warm media, washed twice and resuspended in complete RPMI, plated in a 6 well tissue culture dish at 1×10⁷ cells/well, and incubated in human M-CSF at 20 ng/ml. Cells were incubated for 24 hours at 37° C. to allow monocytes to adhere to the plate. Media was aspirated to remove non-adhered cells, and cells were incubated in fresh media containing 20 ng/ml M-CSF at 37° C. for 6 days, when the vast majority of adhered cells adopted a distinct macrophage morphology by visual inspection. Cells were then stimulated with 1 ng/ml LPS alone or with 10 nM IL-10 (WT or mutant) for 24 hours. Cell supernatants were isolated and levels of TNF-α (Thermo Fisher Scientific), IL-6 (R&D), and CXCL8 (R&D) were measured by ELISA as per the manufacturer’s instruction.

### IFN-γ and Granzyme B Induction by CD8+ T Cells

CD8+ T cells were isolated from human PBMCs using magnetic activated cell sorting (MACS) using MACS LS columns (Miltenyi) in combination with human CD8+ isolation kit (Miltenyi) per the manufacturer’s instructions. The potentiation of and IFN-γ and Granzyme B production by IL-10 was performed as described previously. Briefly, isolated CD8+ T cells were seeded at 2×10⁶ cells/ml in a 6 well tissue culture dish pre-coated with anti-CD3 antibody (clone OKT1) and incubated with soluble anti CD28 antibody (5 µg/ml) for 3 days. Cells were then collected and re-seeded in fresh media at 1×10⁶ cells/ml in a 24 well tissue culture dish with or without 10 nM recombinant IL-10 (WT or mutant) and incubated for an additional 3 days. Cells were then re-stimulated with soluble anti-CD3 (clone OKT1, 1 µg/ml) for 4 hours. Supernatant was then isolated and levels of IFN-γ (Thermo) and Granzyme B (R&D) were measured by ELISA as per the manufacturer’s instructions.

### Example 2

### Cryo-EM Structure of the Hexameric IL-10/IL10R1/IL10Rß Complex

This Example describes the results of experiments performed to determine the crystal structure of the heteromeric receptor complex IL-10/IL10R1/IL10Rß, which in turns helps elucidate the chemistry that drives each of the cytokine-receptor interactions of the heteromeric receptor complex.

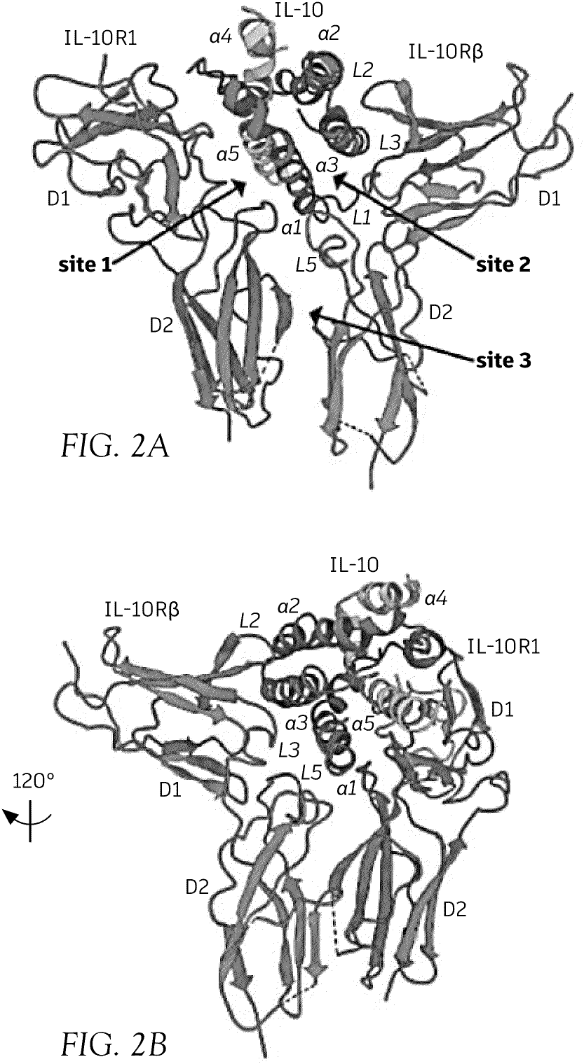

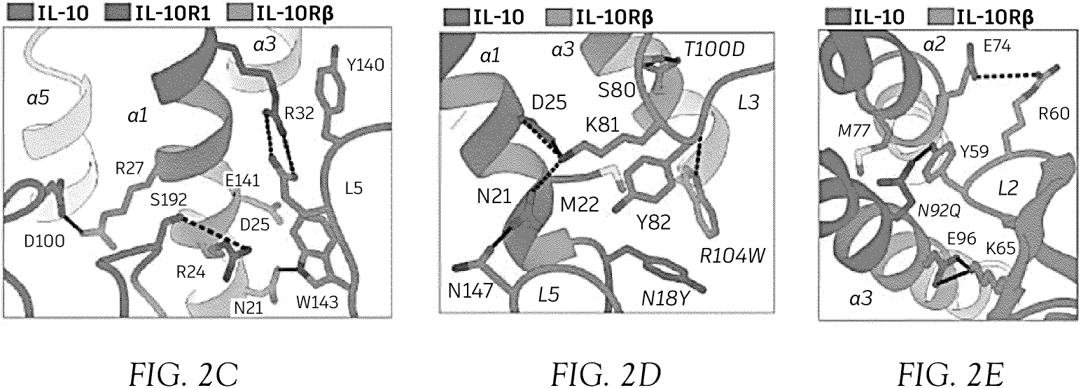

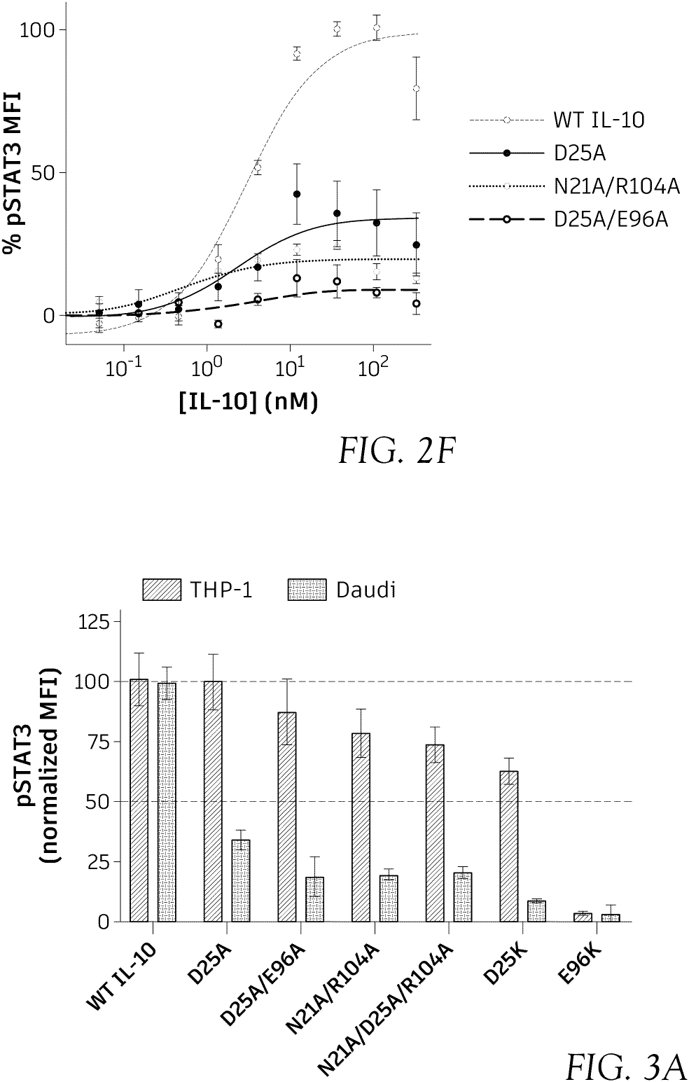

A close inspection of the IL-10/IL-10Rβ binding interface revealed the central contact is formed by 10Rβ-Tyr⁸² in loop L3, which is inserted between IL-10 helices α1 and α3, facilitating Van der Waals contacts primarily with Met²² in helix α1 of IL-10 (FIG. 2D). Previous mutagenesis and modeling studies also suggested that 10Rβ-Tyr⁸² is required for the interaction between IL-10Rβ and IL-10. This key hydrophobic contact is surrounded by a network of polar and electrostatic interactions, mediated in part by IL-10 residues Asn²¹ and Asp²⁵ in helix α1, which engage 10Rβ-Gln⁶³ and10Rβ-Lys⁸¹ , respectively, (FIGS. 2D and 2E). Directly above L3, 10Rβ-Tyr⁵⁹ in loop L2 of IL-10Rβ similarly inserts between IL-10 helices α2 and α3, engaging a several residues in α2, whereas Glu⁹⁶ in α3 of IL-10 engages 10Rβ- Lys⁶⁵ (FIG. 2E). At the front edge of IL-10, loop L5 in D2 of IL-10Rβ forms a “thumb”-like protrusion that extends along the front surface of IL-10 helix α1, facilitating an apparent pi-stacking contact between 10Rβ-Tyr¹⁴⁰ and IL-10 residue Arg³², as well as a hydrogen bond contact between 10Rβ-Asn¹⁴⁷ and Asn²¹ of IL-10 (FIGS. 2C and 2D).

The increased affinity of super-10 for IL-10Rβ was found to result primarily from the N18Y and R104W mutations, both of which are adjacent to Met²² and enhance the hydrophobicity of this central contact site accommodating 10Rβ-Tyr⁸² (FIG. 2D). Meanwhile, the N92Q and T100D mutations facilitate hydrogen bond contacts with 10Rβ-G1n⁶³ and 10Rβ-Ser⁸⁰, respectively, likely enhancing contacts present in the WT IL-10 complex (FIGS. 2D and 2E).

Overall, without being bound to any particular theory, it appears that the affinity enhancing mutations in Super-10 are primarily improving upon pre-existing polar and non-polar contacts made between WT IL-10 and IL-10Rβ, rather than creating novel contact sites.

### Example 3

### Structure-Guided Design of IL-10 Receptor Partial Agonists

This Example describes experiments performed to design IL-10 receptor partial agonists based on the cryo-EM structure of the heteromeric IL-10/IL10R1/IL10Rß complex described in Example 3 above.

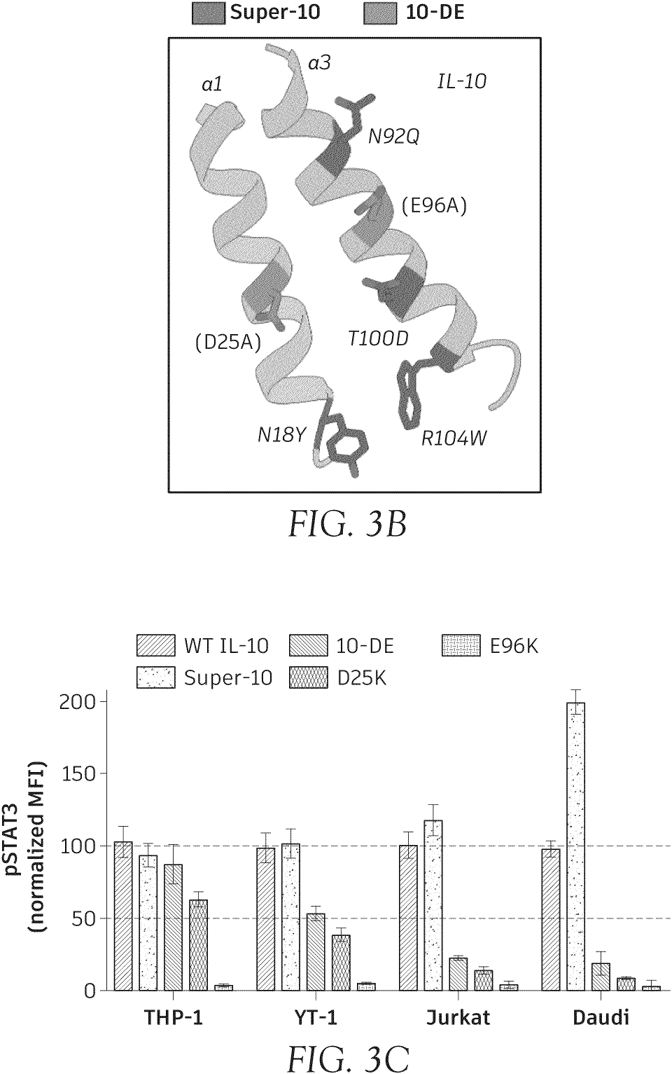

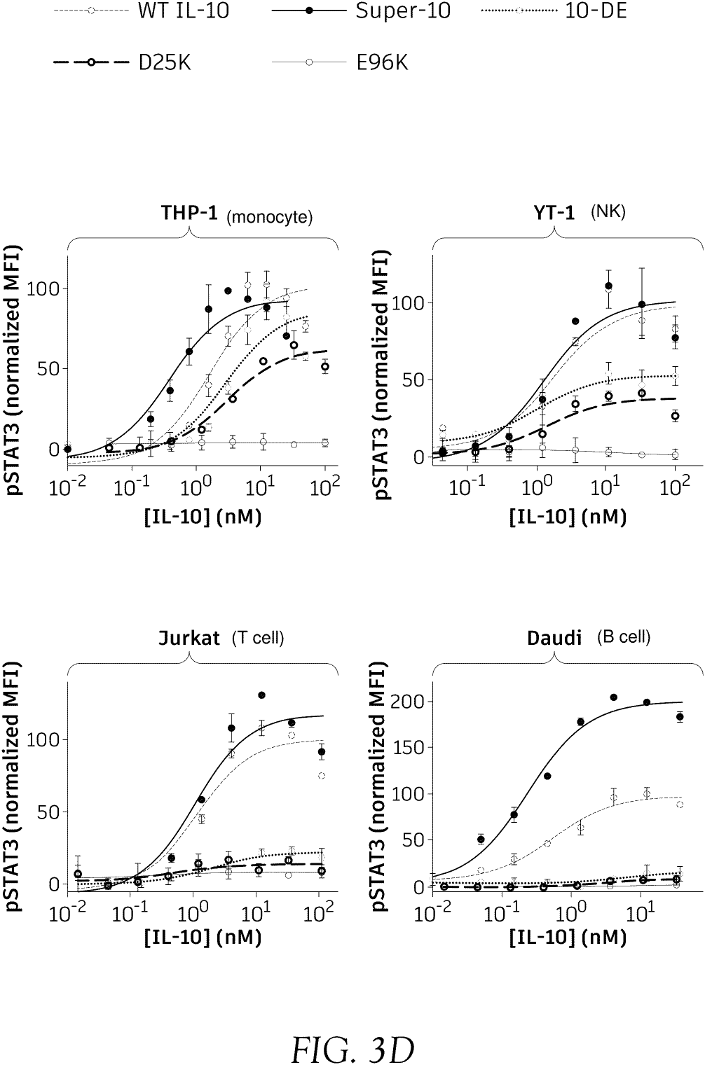

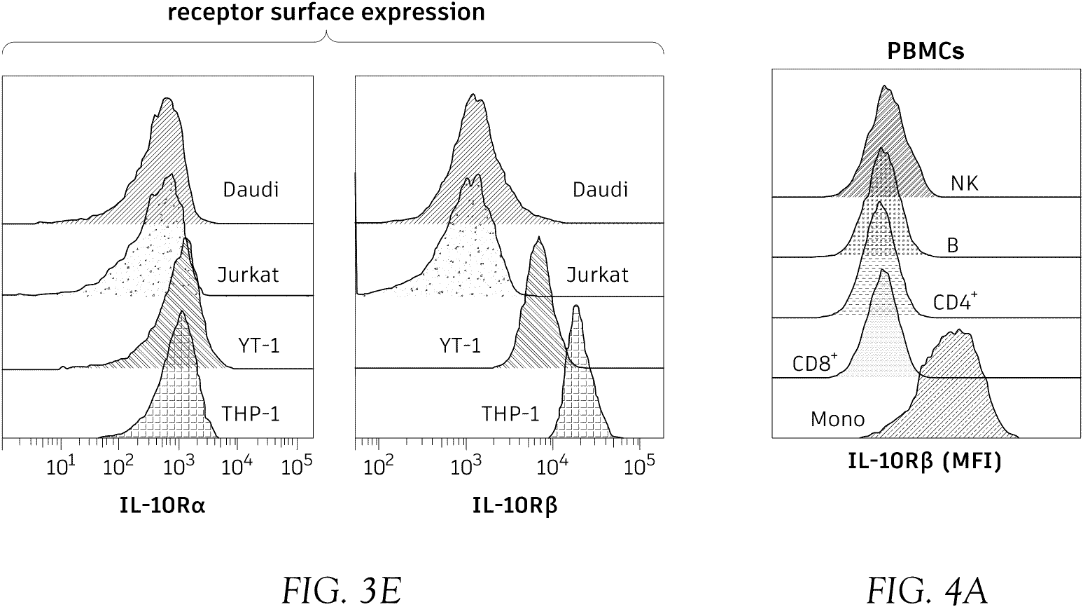

To assess the importance of the IL-10/IL-10Rβ contacts described above for IL-10 signaling, a series of mutations in WT IL-10 predicted to weaken the interaction with IL-10Rβ was designed and evaluated for their ability to initiate signaling via the phosphorylation of STAT3. It was observed that mutation of Asp²⁵ to alanine (D25A) or lysine (D25K) resulted in a substantial reduction in STAT3 activation in B cell-derived Daudi cells, as compared to WT IL-10 (FIG. 2F). Similarly, the IL-10 variants comprising E96K, D25A/E96A, N21A/R104A and D25A/N21A/R104A all elicited negligible STAT3 phosphorylation on these cells, respectively (FIG. 2F). Remarkably, however, all of these variants with the exception of E96K elicited strong elicited STAT3 activation in monocyte-derived THP-1 cells, with the D25A/E96A variant (i.e., “10-DE”) and D25K variant showing the greatest difference in activity between the THP-1 and Daudi cell lines (FIG. 3A.

### Example 4

### IL-10 Variants 10-DE and Super-10 Elicit Cell-Type Selective Signaling

This Example describes experiments performed to demonstrate that an exemplary biased IL-10 variant (10-DE) in accordance with some non-limiting embodiments of the disclosure elicits cell-type selective signaling activity.

In order to generate a more complete picture of how altering the stability of the IL-10/IL-10Rβ interaction influences IL-10 signaling activity across cell types, additional experiments were performed to compare the activity of (i) WT IL-10, (ii) the high affinity agonist super-10, and (iii) the low-affinity partial agonists 10-DE and D25K across a panel of four IL-10 responsive cell lines representing different classes of immune cells (FIG. 3B). Whereas 10-DE variant and D25K variant elicited no activity in either B cell-derived Daudi cells or T cell derived Jurkat cells, they elicited ~50% activity in natural killer (NK) cell-derived YT-1 cells and nearly full STAT3 activation in myeloid THP-1 cells (FIGS. 3A-3D). By contrast, super-10 showed substantially higher Eₘₐₓ relative to WT IL-10 in both Daudi and Jurkat cells, but similar signaling as WT IL-10 in YT-1 and THP-1 cells (FIGS. 3A-3D). Thus, the ratio of super-10 to 10-DE STAT3 Eₘₐₓ varied considerably, from ~1:1 in THP-1 cells to over 20:1 in Daudi cells, representing substantial differences in signaling plasticity between these cell lines.

Comparison of IL-10Rα and IL-10Rβ surface expression across these diverse cell lines revealed that the level of IL-10Rα and IL-10Rβ correlated well with the relative strength of STAT3 signaling induced by the engineered IL-10 variants (FIG. 3E). The finding that super-10 can increase the signaling Eₘₐₓ relative to WT IL-10 on certain cell lines suggests that assembly of the IL-10 signaling complex on these cells is limited by low IL-10R expression, which can be compensated for by enhancing the affinity of the IL-10-IL-10Rβ interaction. Conversely, other cell types can maintain robust STAT3 responses even to the low-affinity IL-10 partial agonists such as 10-DE and D25K, due to their elevated IL-10R expression (FIG. 3C). This observation suggests the presence of a functional window that can be exploited with engineered agonists to achieve narrowed IL-10 cell-type specificity.

Comparison of IL-10Rα and IL-10Rβ surface expression across these diverse cell lines revealed that the level of IL-10Rα and IL-10Rβ correlated well with the relative strength of STAT3 signaling induced by the engineered IL-10 variants (fig. S6, D and E). Thus, extent of STAT3 activation at saturating IL-10 concentration can be modulated by a combination of receptor expression and ligand affinity (FIG. 3C). The finding that super-10 can increase the signaling Emax relative to WT IL-10 on certain cell lines suggests that assembly of the IL-10 signaling complex on these cells is limited by low IL-10R expression, which can be compensated for by enhancing the affinity of the IL-10/IL-10Rβ interaction. Conversely, other cell types can maintain robust STAT3 responses even to the low-affinity IL-10 partial agonists such as 10-DE and D25K, due to their elevated IL-10R expression (FIG. 3C). This observation suggests the presence of a functional window that can be exploited with engineered agonists to achieve narrowed IL-10 cell-type specificity.

### Example 5

### IL-10 Variants 10-DE and D25K Uncouple Anti-Inflammatory and Immunostimulatory Functions of IL-10 Ex Vivo

This Example describes the results from experiments performed to demonstrate that the biased IL-10 variants 10-DE and Super-10 uncouple anti-inflammatory and immunostimulatory functions of IL-10. These experiments were designed to address the question as to whether natural differences in IL-10 signaling plasticity could be exploited to ‘tune’ the functional pleiotropy of IL-10 in primary immune cell populations.

As explained above, IL-10 elicits both anti-inflammatory and immunostimulatory effects in vivo due to its ability to initiate signaling in multiple immune cell types. The anti-inflammatory effects of IL-10 are primarily due to suppression of monocyte and macrophage activity, whereas IL-10 promotes pro-inflammatory IFN-y production via stimulation of CD8+ T cells. Experiments described below were performed to address the question of whether natural differences in IL-10 signaling plasticity could be exploited to ‘tune’ the functional pleiotropy of IL-10 in primary immune cell populations.

Analysis of human peripheral blood mononuclear cells (PBMCs) by flow cytometry revealed that IL-10Rβ is highly expressed in primary monocytes, but much more lowly expressed in NK cells, B cells, and T cells, suggesting that monocytes may exhibit more robust IL-10 signaling responses (FIG. 4A). Indeed, despite vast differences in affinity for IL-10Rβ, both 10-DE and Super-10 variants were found to exhibit equivalent levels of STAT3 activation as WT IL-10 in primary monocytes (FIGS. 4B and 6B). By contrast, 10-DE variant exhibited significantly reduced signaling in NK cells, B cells and T cells, eliciting between 35% and 50% STAT3 activation relative to WT IL-10 in these populations (FIGS. 4B and 6B). Super-10 however was found to elicit strongly enhanced STAT3 activation in NK cells, but only slightly elevated signaling in B and T cells, suggesting that additional factors may also limit further increases in STAT3 activation in those cell types (FIG. 4B). Overall, these data show that while Super-10 exhibits moderate NK cell-biased activity, the partial agonist 10-DE exhibits pronounced monocyte-biased signaling in primary human cells.

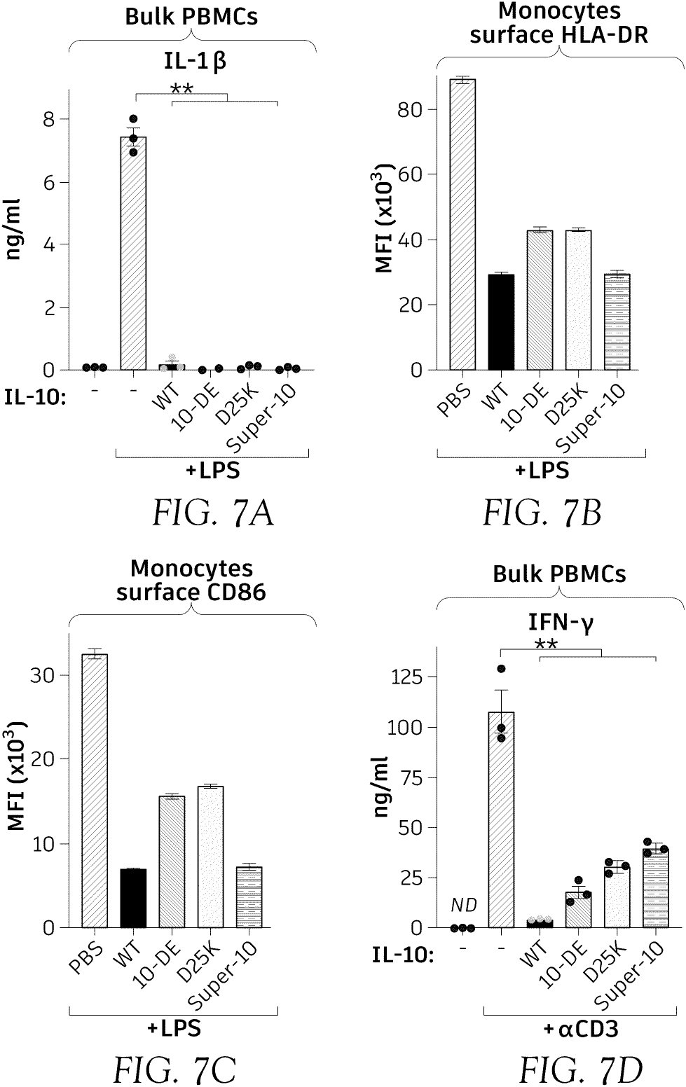

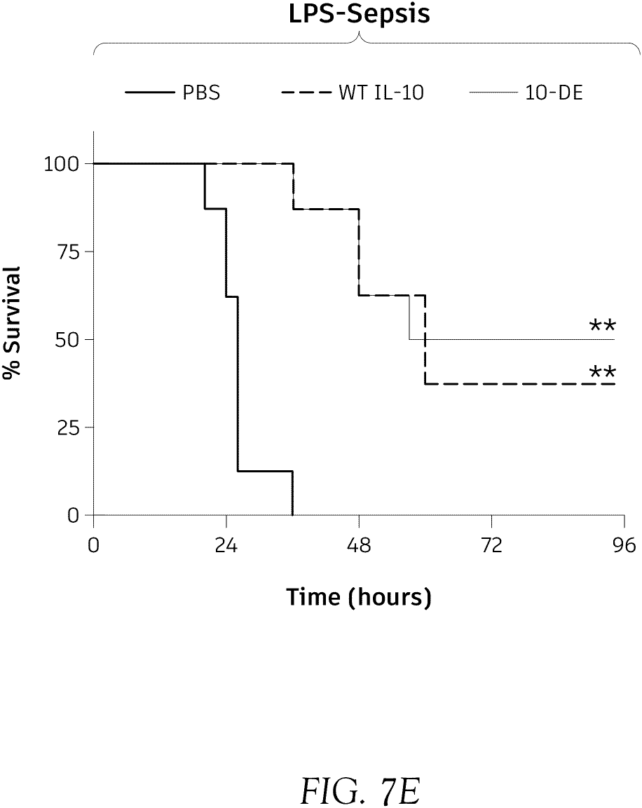

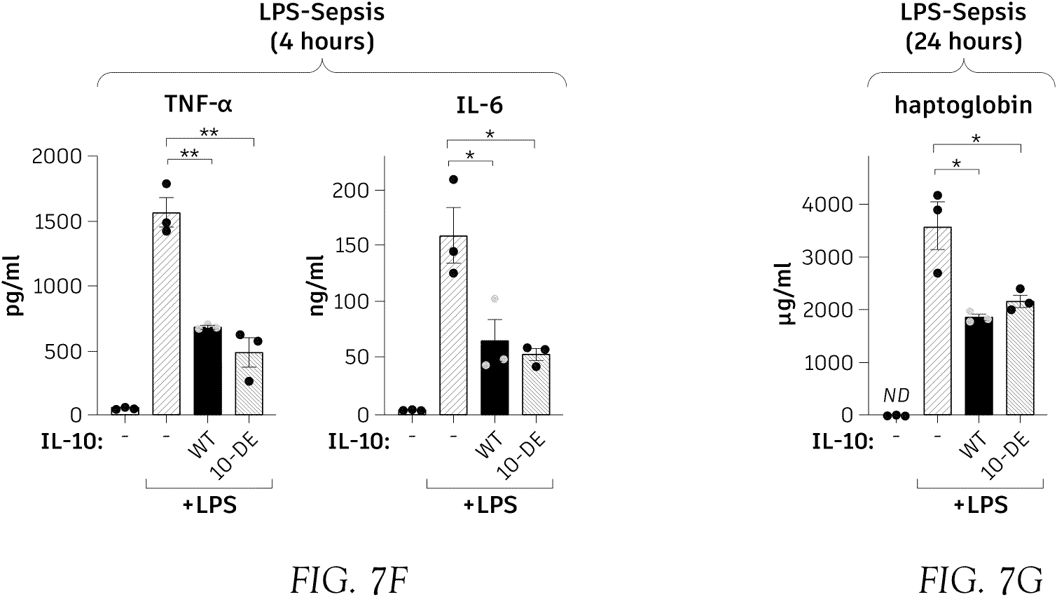

To test whether the level of STAT3 activation induced by 10-DE and D25K variants in monocytes was sufficient to drive anti-inflammatory functions of IL-10, additional experiments were perform to stimulate bulk human PBMCs with bacterial lipopolysaccharide (LPS) in the presence or absence of these variants. Notably, both 10-DE variant and D25K variant inhibited the production of the pro-inflammatory cytokines IL-1β, TNF-α and IL-6, as well as the chemokine IL-8, to a similar extent as WT IL-10 and Super-10 variant (FIGS. 4C and 7A). In addition, 10-DE and D25K retained the ability to suppress IFN-γ production in bulk PBMCs activated with anti-CD3 antibodies (FIG. 7D), and also downregulate surface expression of MHC class II and the co-stimulatory ligand CD86 on peripheral monocytes (FIGS. 4D, 7D, and 7C). Importantly, 10-DE variant and D25K variant also strongly abrogated LPS-induced TNF-α, IL-6, and IL-8 production in isolated monocyte-derived macrophages (FIG. 4E), the key target for IL-10 in autoimmune and auto-inflammatory diseases.

Additional experiments were also performed to assess whether the myeloid-biased activity of 10-DE variant was sufficient to suppress systemic inflammation in a mouse model of LPS-induced sepsis. Importantly, it was observed that a single injection of 10-DE variant significantly promoted survival in this model, to the same extent as WT IL-10 (FIG. 7E). Moreover, it was also observed that 10-DE variant blunted the elevation of TNF-α, IL-6, and haptoglobin, key markers of systemic inflammation (FIGS. 7F and 7G).

### Myeloid-Biased IL-10 Variants Have Reduced Capacity to Promote Inflammatory T Cell Functions

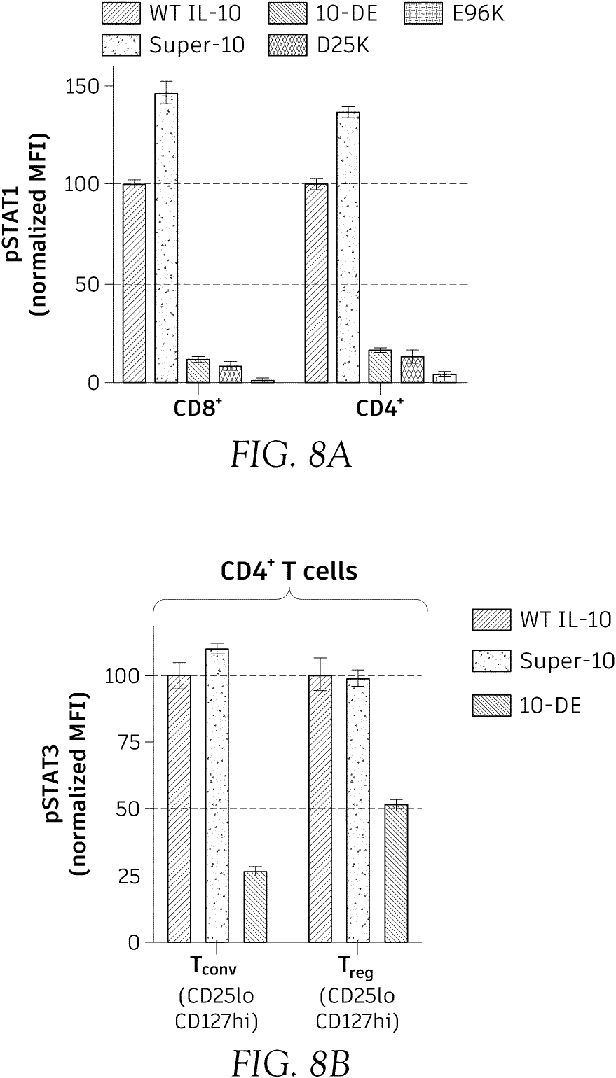

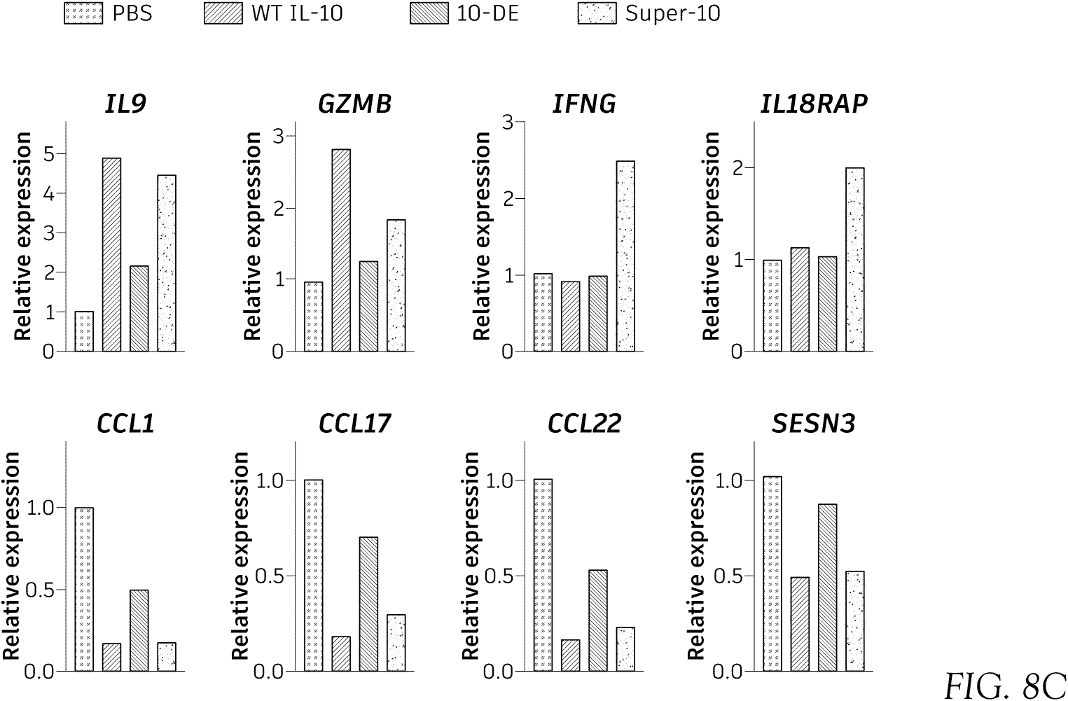

IL-10 has been known to induce pro-inflammatory IFN-γ production, and recent studies have suggested that this occurs primarily through the potentiation of CD8+ T cell activity. In order to analyze the effects of the engineered IL-10 variants described herein on CD8⁺ T-cells, RNA sequencing (RNA-seq) analysis was performed on TCR-stimulated CD8⁺ T cells cultured with WT IL-10, super-10, or 10-DE for twenty-four hours (FIG. 8C).

The expression changes induced by super-10 largely matched the pattern of observed with WT IL-10, albeit with less upregulation of some IL-10 target genes, such as GZMB, IL22, and SOCS3, and enhanced induction of others such as IFNG and IL18RAP (FIG. 8C), potentially reflecting the increased STAT1 activation elicited by super-10 compared to WT IL-10 in these cells (FIG. 8A). By contrast, the partial agonist 10-DE elicited a pattern of substantially weaker transcriptional changes across both upregulated and downregulated IL-10 target genes, including reduced expression of pro-inflammatory genes such as GZMB (FIG. 8C). Consistent with this, both 10-DE and D25K failed to potentiate granzyme B, IFN-γ, and IL-9 production by activated CD8⁺ T cells, in contrast to WT IL-10 and super-10 (FIG. 4F). Thus, although the myeloid-biased IL-10 variants retained the ability to suppress monocyte and macrophage activation, they showed substantially reduced capacity to potentiate inflammatory T cell activity, thereby uncoupling the major anti-inflammatory and pro-inflammatory functions of IL-10 (FIG. 4G).

The experimental data described herein showed that the myeloid-biased signaling induced by engineered IL-10 partial agonists can uncouple important aspects of IL-10 function. The differential IL-10 receptor expression between myeloid and lymphocyte populations may therefore represent a natural mechanism to provide functional specificity to otherwise pleiotropic IL-10 responses, a feature that can be exploited using engineered agonists as demonstrated herein. For example, the partial agonists 10-DE and D25K retained the anti-inflammatory functions of IL-10, consistent with their capacity to activate STAT3 in myeloid cells to a similar extent as WT IL-10 (FIGS. 4B-4E and 7A-7G). By contrast, these variants had significantly diminished capacity to potentiate inflammatory IFN-γ production by both CD4⁺ and CD8⁺ T cells, consistent with their reduced ability to activate both STAT3 and STAT1 in these cells (FIGS. 4B, 4F; 6B, and 8A). Meanwhile, super-10 elicited stronger induction of IFN-γ by both CD4⁺ and CD8⁺ T cells relative to WT IL-10, despite eliciting a similar STAT3 response (FIGS. 4F, 6B, and 8C ). This finding is likely explained by the observation that super-10 elicited substantially stronger STAT1 activation in these cells (FIG. 8A), consistent with STAT1 having been suggested to mediate the IFN-γ-inducing effects of IL-10.

As discussed in further details below, the natural ability of IL-10 to potently inhibit the production of pro-inflammatory cytokines like IL-6 and TNF-α in vivo makes it a good therapeutic candidate for the treatment of several autoimmune diseases, including rheumatoid arthritis, inflammatory bowel disease (IBD), and Type 1 diabetes. It was observed that the observation that IL-10 administration elevates IFN-γ levels in humans has presented a barrier to the use of IL-10 in these contexts. Therefore, the ability of engineered IL-10 partial agonists to suppress inflammatory monocyte and macrophage activation without stimulating IFN-γ production by CD4⁺ and CD8⁺ T cells has significant implications for the clinical use of IL-10 in these settings, providing a blueprint for unlocking the full therapeutic potential of IL-10..

While particular alternatives of the present disclosure have been disclosed, it is to be understood that various modifications and combinations are possible and are contemplated within the true spirit and scope of the appended claims. There is no intention, therefore, of limitations to the exact abstract and disclosure herein presented.

### REFERENCES

1. R. Medzhitov, Origin and physiological roles of inflammation. Nature 454, 428-435 (2008).

2. M. E. Kotas, R. Medzhitov, Homeostasis, inflammation, and disease susceptibility. Cell 160, 816-827 (2015).

3. K. W. Moore, R. de Waal Malefyt, R. L. Coffman, A. O′Garra, Interleukin-10 and the interleukin-10 receptor. Annu. Rev. of Immunol. 19, 683-765 (2001).

4. W. Ouyang, S. Rutz, N. K. Crellin, P. A. Valdez, S. G. Hymowitz, Regulation and functions of the IL-10 family of cytokines in inflammation and disease. Annu. Rev. of Immunol. 29, 71-109 (2011).

5. D. F. Fiorentino, A. Zlotnik, T. R. Mosmann, M. Howard, A. O′Garra, IL-10 inhibits cytokine production by activated macrophages. J. Immunol. 147, 3815-3822 (1991).

6. E. Zigmond et al., Macrophage-restricted interleukin-10 receptor deficiency, but not IL-10 deficiency, causes severe spontaneous colitis. Immunity 40, 720-733 (2014).

7. D. S. Shouval et al., Interleukin-10 receptor signaling in innate immune cells regulates mucosal immune tolerance and anti-inflammatory macrophage function. Immunity 40, 706-719 (2014).

8. R. Kuhn, J. Lohler, D. Rennick, K. Rajewsky, W. Muller, Interleukin-10-deficient mice develop chronic enterocolitis. Cell 75, 263-274 (1993).

9. K. R. Engelhardt, B. Grimbacher, IL-10 in humans: lessons from the gut, IL-10/IL-10 receptor deficiencies, and IL-10 polymorphisms. Curr. Top. Microbiol. and Immunol. 380, 1-18 (2014).

10. M. Saraiva, P. Vieira, A. O′Garra, Biology and therapeutic potential of interleukin-10. The J. Exp. Med 217, e20190418 (2020).

11. S. Fujii, K. Shimizu, T. Shimizu, M. T. Lotze, Interleukin-10 promotes the maintenance of antitumor CD8(+) T-cell effector function in situ. Blood 98, 2143-2151 (2001).

12. J. B. Mumm etal., IL-10 elicits IFNgamma-dependent tumor immune surveillance. Cancer cell 20, 781-796 (2011).

13. I. H. Chan et al., The Potentiation of IFN-gamma and Induction of Cytotoxic Proteins by Pegylated IL-10 in Human CD8 T Cells. J. Interferon Cytokine Res. 35, 948-955 (2015).

14. H. Tilg etal., Treatment of Crohn’s disease with recombinant human interleukin 10 induces the proinflammatory cytokine interferon gamma. Gut 50, 191-195 (2002).

15. K. Josephson, N. J. Logsdon, M. R. Walter, Crystal structure of the IL-10/IL-10R1 complex reveals a shared receptor binding site. Immunity 15, 35-46 (2001).

16. G. Chao et al., Isolating and engineering human antibodies using yeast surface display. Nat. Protoc. 1, 755-768 (2006).

17. K. Josephson et al., Design and analysis of an engineered human interleukin-10 monomer. J. Biol. 275, 13552-13557 (2000).

18. S. I. Yoon et al., Structure and mechanism of receptor sharing by the IL-10R2 common chain. Structure 18, 638-648 (2010).

19. J. L. Mendoza et al., Structure of the IFNgamma receptor complex guides design of biased agonists. Nature 567, 56-60 (2019).

20. J. L. Mendoza et al., The IFN-lambda-IFN-lambdaR1-IL-10Rbeta Complex Reveals Structural Features Underlying Type III IFN Functional Plasticity. Immunity 46, 379-392 (2017).

21. Y. Xiao et al., Comprehensive mutation screening for 10 genes in Chinese patients suffering very early onset inflammatory bowel disease. World J. Gastroenterol. 22, 5578-5588 (2016).

22. D. N. Mastronarde, Automated electron microscope tomography using robust prediction of specimen movements. J. Struct. Biol. 152, 36-51 (2005).

23. S. Q. Zheng et al., MotionCor2: anisotropic correction of beam-induced motion for improved cryo-electron microscopy. Nat. Methods 14, 331-332 (2017).

24. A. Punjani, J. L. Rubinstein, D. J. Fleet, M. A. Brubaker, cryoSPARC: algorithms for rapid unsupervised cryo-EM structure determination. Nat. Methods 14, 290-296 (2017).

25 Tan, Y.Z., Baldwin, P.R., Davis, J.H., Williamson, J.R., Potter, C.S., Carragher, B. and Lyumkis, D., 2017. Addressing preferred specimen orientation in single-particle cryo-EM through tilting. Nature methods, 14(8), p.793.

26. T. D. Goddard et al., UCSF ChimeraX: Meeting modern challenges in visualization and analysis. Protein Sci. 27, 14-25 (2018).

27. P. Emsley, B. Lohkamp, W. G. Scott, K. Cowtan, Features and development of Coot. Acta Cryst. 66, 486-501 (2010).

28. P. D. Adams et al., PHENIX: a comprehensive Python-based system for macromolecular structure solution. Acta Crystallogr D. 66, 213-221 (2010).

29. P. V. Afonine et al., Towards automated crystallographic structure refinement with phenix.refine. Acta Crystallogr D. 68, 352-367 (2012).

30. P. Brodin et al., Variation in the human immune system is largely driven by non-heritable influences. Cell 160, 37-47 (2015).

31. Dobin A, Davis C A, Schlesinger F, et al. STAR: ultrafast universal RNA-seq aligner. Bioinformatics. 29(1): 15-21 (2013).

32. Mortazavi, A., Williams, B., McCue, K. et al. Mapping and quantifying mammalian transcriptomes by RNA-Seq. Nat Methods. 5, 621-628 (2008).

What is claimed is:

1. A recombinant polypeptide comprising:
an amino acid sequence having at least 70% sequence identity to an interleukin-10 (IL-10) polypeptide having the amino acid sequence of SEQ ID NO: 1 or SEQ ID NO: 16;
and further comprising one or more amino acid substitution at a position corresponding to an amino acid residue selected from the group consisting of X25, X14, X18, X24, X28, X74, X90, X92, X96, X100 and X104 of SEQ ID NO: 1 or SEQ ID NO: 16.

2. The recombinant polypeptide of claim 1, wherein the one or more amino acid substitution is at a position corresponding to an amino acid residue selected from the group consisting of X25, and X96 of SEQ ID NO: 1 or SEQ ID NO: 16.

3. The recombinant polypeptide of any one of claims 1 to 2, wherein the amino acid sequence further comprising at least one additional amino acid substitution at a position corresponding to an amino acid residue selected from the group consisting of X21, X22, X32, and X93 of SEQ ID NO: 1 or SEQ ID NO: 16.

4. The recombinant polypeptide of any one of claims 1 to 3, wherein the recombinant polypeptide has an altered binding affinity for IL-10 receptor beta (IL-10Rβ) compared to binding affinity of a reference IL-10 polypeptide lacking the one or more amino acid substitution.

5. The recombinant polypeptide of claim 4, wherein the recombinant polypeptide has a reduced binding affinity for IL-10Rβ compared to binding affinity of the reference IL-10 polypeptide.

6. The recombinant polypeptide of any one of claims 4 to 5, wherein the recombinant polypeptide has a reduced capability to stimulate STAT3 signaling compared to the reference IL-10 polypeptide.

7. The recombinant polypeptide of claim 4, wherein the recombinant polypeptide has an increased binding affinity for IL-10Rβ compared to binding affinity of the reference IL-10 polypeptide.

8. The recombinant polypeptide of any one of claims 4 to 7, wherein the recombinant polypeptide confers a cell-type biased signaling of the downstream signal transduction mediated through IL-10 compared to the reference IL-10 polypeptide.

9. The recombinant polypeptide of any one of claims 1 to 8, wherein the one or more amino acid substitution is independently selected from the group consisting of an alanine substitution, an aspartic acid substitution, a histidine substitution, a glutamic acid substitution, a lysine substitution, a serine substitution, a tryptophan substitution, a tyrosine substitution, a valine substitution, and combinations of any thereof.

10. The recombinant polypeptide of any one of claims 1 to 9, wherein the one or more amino acid substitution is at a position corresponding to an amino acid residue selected from the group consisting of D25, H14, N18, R24, D28, E74, H90, N92, E96, T100 and R104 of SEQ ID NO: 1 or SEQ ID NO: 16.

11. The recombinant polypeptide of claim 10, wherein the one or more amino acid substitution is at a position corresponding to an amino acid residue selected from the group consisting of D25 and E96 of SEQ ID NO: 1 or SEQ ID NO: 16.

12. The recombinant polypeptide of any one of claims 10 to 11, wherein the amino acid sequence further comprising at least one additional amino acid substitution at a position corresponding to an amino acid residue selected from the group consisting of N21, M22, R32, and S93 of SEQ ID NO: 1 or SEQ ID NO: 16.

13. The recombinant polypeptide of any one of claims 1 to 12, comprising an amino acid sequence having at least 70% sequence identity to SEQ ID NO: 1 or SEQ ID NO: 16, and further comprising the amino acid substitutions corresponding to the following amino acid substitutions:
a) N18Y/N92Q/T100D/R104W;
b) N18Y/N21H/N92Q/E96D/T100V/R104W;
c) N18Y/N21H/E96H/T100V/R104W;
d) N18Y/D25A/N92Q/T100D/R104W;
e) N18Y/D25K/N92Q/T100D/R104W; and
f) N18Y/D25A/N92Q/E96A/T100D/R104W.

14. The recombinant polypeptide of any one of claims 1 to 12, comprising an amino acid sequence having at least 70% sequence identity to SEQ ID NO: 1 or SEQ ID NO: 16, and further comprising the amino acid substitutions corresponding to the following amino acid substitutions:
a) a) D25A;
b) D25K;
c) E96A;
d) E96K;
e) D25A/E96A;
f) N21A/R104A;
g) N21A/D25A;
h) N21A/D25A/E96A; and
i) N21A/M22A/D25A.

15. A recombinant nucleic acid molecule comprising a nucleic acid sequence encoding a polypeptide that comprises an amino acid sequence having at least 70% sequence identity to the amino acid sequence of the polypeptide of any one of 1-14 and 23.

16. The nucleic acid molecule of claim 15, wherein the nucleic acid sequence is operably linked to a heterologous nucleic acid sequence.

17. The nucleic acid molecule of any one of claims 15-16, wherein the nucleic acid molecule is incorporated into an expression cassette or an expression vector.

18. A recombinant cell comprising:
a) a recombinant polypeptide according to any one of claims 1-14 and 23; and/or
b) a recombinant nucleic acid of any one of claims 15-17.

19. The recombinant cell of claim 18, wherein the recombinant cell is a eukaryotic cell.

20. The recombinant cell of claim 19, wherein the eukaryotic cell is a mammalian cell.

21. A cell culture comprising at least one recombinant cell of any one of claims 18, and a culture medium.

22. A method for producing a polypeptide comprising:
a) providing one or more recombinant cells of any one of claim 18; and
b) culturing the one or more recombinant cells in a culture medium such that the cells produce the polypeptide encoded by the recombinant nucleic acid molecule.

23. The method of claim 22, further comprising isolating and/or purifying the produced polypeptide.

24. The method of any one of claims 22-23, further comprising structurally modifying the produced polypeptide to increase half-life.

25. The method of claim 24, wherein said modification comprises one or more alterations selected from the group consisting of fusion to a human Fc antibody fragment, fusion to albumin, and PEGylation.

26. A recombinant polypeptide produced by the method of any one of claims 22.

27. A pharmaceutical composition comprising a pharmaceutically acceptable carrier and:
a) a recombinant polypeptide according to any one of claims 1-14 and 23;
b) a recombinant nucleic acid of any one of claims 15-17; and/or
c) a recombinant cell of any one of claims 18.

28. The pharmaceutical composition of claim 27, wherein the composition comprises a recombinant polypeptide according to any one of claims 1-14 and 23, and a pharmaceutically acceptable carrier.

29. The pharmaceutical composition of claim 27, wherein the composition comprises a recombinant nucleic acid according to any one of claims 15-17, and a pharmaceutically acceptable carrier.

30. The pharmaceutical composition of claim 27, wherein the composition comprises a recombinant cell according to any one of claims 16-20, and a pharmaceutically acceptable carrier.

31. A method for modulating IL-10-mediated signaling in a subject, the method comprising administering to the subject a composition comprising:
a) a recombinant polypeptide according to any one of claims 1-14 and 23;
b) a recombinant nucleic acid of any one of claims 15-17;
c) a recombinant cell of any one of claims 18-20; and/or
d) a pharmaceutical composition claim 27-30.

32. A method for the treatment of a health condition in a subject in need thereof, the method comprising administering to the subject a composition comprising:
a) a recombinant polypeptide according any one of claims 1-14 and 23;
b) a recombinant nucleic acid of any one of claims 15-17;
c) a recombinant cell of any one of claims 18-20; and/or
d) a pharmaceutical composition of any one of claims 27-30.

33. The method of any one of claims 31 to 32, wherein the administered composition confers a cell-type biased signaling of the downstream signal transduction mediated through IL-10 compared to a reference IL-10 polypeptide lacking the one or more amino acid substitution.

34. The method of claim 33, wherein the cell-type biased IL-10 signaling comprises a reduction of STAT1- or STAT3-mediated pro-inflammatory function in B cells, T cells, and NK cells while substantially retaining its STAT3-mediated anti-inflammatory function in monocytes and macrophages.

35. The method of claim 34, wherein the STAT3-mediated signaling is determined by an assay selected from the group consisting of by a gene expression assay, a phospho-flow signaling assay, and an enzyme-linked immunosorbent assay (ELISA).

36. The method of any one of claims 34-35, wherein the STAT3-mediated pro-inflammatory function is selected from the group consisting of cytokine production, chemokine production, immune cell proliferation, and immune cell recruitment.

37. The method of any one of claims 34-36, wherein the STAT3-mediated pro-inflammatory function is reduced from about 20% to about 100%.

38. The method of any one of claims 32-37, wherein the administered composition results in a reduced capacity to induce expression of a pro-inflammatory gene selected from IFN-y, granzyme B, granzyme A, perforin, TNF-α, GM-CSF, and MIP1α in the subject.

39. The methods of claim 32, wherein the administered composition stimulates expression of interferon gamma (INFy) in CD8+ T cells.

40. The method of claim 39, wherein the health condition is a cancer.

41. A kit for modulating IL-10-mediated signaling in a subject, or treating a health condition in a subject in need thereof, the system comprising:
a) a recombinant polypeptide according to any one of claims 1-14 and 23;
b) a recombinant nucleic acid of any one of claims 15-17;
c) a recombinant cell of any one of claims 18-20; and/or
d) a pharmaceutical composition of any one of claims 27-30.


SEQUENCE LISTING (32 sequences)

SEQ ID NO: 1 - protein, 160 aa, Homo sapiens
  MISC_FEATURE: Wild-type mature human IL-10
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 2 - protein, 160 aa, Artificial Sequence
  Synthetic construct
  MISC_FEATURE: 10_clone 5.1 (super-10)(N18Y/N92Q/T100D/R104W)
SPGQGTQSENSCTHFPGYLPNMLRDLRDAFSRVKTFFQMKDQLDNLLLKESLLEDFKGYLGCQALSEMIQFYLEEVMPQAENQDPDIKAHVQSLGENLKDLRLWLRRCHRFLPCENKSKAVEQVKNAFNKLQEKGIYKAMSEFDIFINYIEAYMTMKIRN

SEQ ID NO: 3 - protein, 160 aa, Artificial Sequence
  Synthetic construct
  MISC_FEATURE: 10_clone 5.2 (N18Y/N21H /N92Q/E96D/T100V R104W)
SPGQGTQSENSCTHFPGYLPHMLRDLRDAFSRVKTFFQMKDQLDNLLLKESLLEDFKGYLGCQALSEMIQFYLEEVMPQAENQDPDIKAHVQSLGDNLKVLRLWLRRCHRFLPCENKSKAVEQVKNAFNKLQEKGIYKAMSEFDIFINYIEAYMTMKIRN

SEQ ID NO: 4 - protein, 160 aa, Artificial Sequence
  Synthetic construct
  MISC_FEATURE: 10_clone 5.3 (N18Y/N21H/E96H T100V/R104W)
SPGQGTQSENSCTHFPGYLPHMLRDLRDAFSRVKTFFQMKDQLDNLLLKESLLEDFKGYLGCQALSEMIQFYLEEVMPQAENQDPDIKAHVNSLGHNLKVLRLWLRRCHRFLPCENKSKAVEQVKNAFNKLQEKGIYKAMSEFDIFINYIEAYMTMKIRN

SEQ ID NO: 5 - protein, 160 aa, Artificial Sequence
  Synthetic construct
  MISC_FEATURE: IL-10 variant BS63 (D25A)
SPGQGTQSENSCTHFPGNLPNMLRALRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 6 - protein, 160 aa, Artificial Sequence
  Synthetic construct
  MISC_FEATURE: IL-10 variant BS64 (N21A/R104A)
SPGQGTQSENSCTHFPGNLPAMLRDLRDAFSRVKTFFQMKDQLDNLLLKESLLEDFKGYLGCQALSEMIQFYLEEVMPQAENQDPDIKAHVNSLGENLKTLRLALRRCHRFLPCENKSKAVEQVKNAFNKLQEKGIYKAMSEFDIFINYIEAYMTMKIRN

SEQ ID NO: 7 - protein, 160 aa, Artificial Sequence
  Synthetic construct
  MISC_FEATURE: IL-10 variant BS65 (10-DE) (D25A/E96A)
SPGQGTQSENSCTHFPGNLPNMLRALRDAFSRVKTFFQMKDQLDNLLLKESLLEDFKGYLGCQALSEMIQFYLEEVMPQAENQDPDIKAHVNSLGANLKTLRLRLRRCHRFLPCENKSKAVEQVKNAFNKLQEKGIYKAMSEFDIFINYIEAYMTMKIRN

SEQ ID NO: 8 - protein, 160 aa, Artificial Sequence
  Synthetic construct
  MISC_FEATURE: IL-10 variant BS66 (N21A/D25A)
SPGQGTQSENSCTHFPGNLPAMLRALRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 9 - protein, 160 aa, Artificial Sequence
  Synthetic construct
  MISC_FEATURE: IL-10 variant BS68 (N21A/D25A/E96A)
SPGQGTQSENSCTHFPGNLPAMLRALRDAFSRVKTFFQMKDQLDNLLLKESLLEDFKGYLGCQALSEMIQFYLEEVMPQAENQDPDIKAHVNSLGANLKTLRLRLRRCHRFLPCENKSKAVEQVKNAFNKLQEKGIYKAMSEFDIFINYIEAYMTMKIRN

SEQ ID NO: 10 - protein, 160 aa, Artificial Sequence
  Synthetic construct
  MISC_FEATURE: IL-10 variant (N21A/D25A/ R104A)
SPGQGTQSENSCTHFPGNLPAMLRALRDAFSRVKTFFQMKDQLDNLLLKESLLEDFKGYLGCQALSEMIQFYLEEVMPQAENQDPDIKAHVNSLGENLKTLRLALRRCHRFLPCENKSKAVEQVKNAFNKLQEKGIYKAMSEFDIFINYIEAYMTMKIRN

SEQ ID NO: 11 - protein, 160 aa, Artificial Sequence
  Synthetic construct
  MISC_FEATURE: IL-10 variant (D25K)
SPGQGTQSENSCTHFPGNLPNMLRKLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 12 - protein, 160 aa, Artificial Sequence
  Synthetic construct
  MISC_FEATURE: IL-10 variant (E96K)
SPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGKNLKTLRLRLRRCHRFLPCENKSKAVEQVKNAFNKLQEKGIYKAMSEFDIFINYIEAYMTMKIRN

SEQ ID NO: 13 - protein, 160 aa, Artificial Sequence
  Synthetic construct
  MISC_FEATURE: IL-10 variant (N18Y/D25A/N92Q/T100D/R104W)
SPGQGTQSENSCTHFPGYLPNMLRALRDAFSRVKTFFQMKDQLDNLLLKESLLEDFKGYLGCQALSEMIQFYLEEVMPQAENQDPDIKAHVQSLGENLKDLRLWLRRCHRFLPCENKSKAVEQVKNAFNKLQEKGIYKAMSEFDIFINYIEAYMTMKIRN

SEQ ID NO: 14 - protein, 160 aa, Artificial Sequence
  Synthetic construct
  MISC_FEATURE: IL-10 variant (N18Y/D25K/N92Q/T100D/R104W)
SPGQGTQSENSCTHFPGYLPNMLRKLRDAFSRVKTFFQMKDQLDNLLLKESLLEDFKGYLGCQALSEMIQFYLEEVMPQAENQDPDIKAHVQSLGENLKDLRLWLRRCHRFLPCENKSKAVEQVKNAFNKLQEKGIYKAMSEFDIFINYIEAYMTMKIRN

SEQ ID NO: 15 - protein, 160 aa, Artificial Sequence
  Synthetic construct
  MISC_FEATURE: IL-10 variant (N18Y/D25A/N92Q/E96A/T100D/R104W)
SPGQGTQSENSCTHFPGYLPNMLRALRDAFSRVKTFFQMKDQLDNLLLKESLLEDFKGYLGCQALSEMIQFYLEEVMPQAENQDPDIKAHVQSLGANLKDLRLWLRRCHRFLPCENKSKAVEQVKNAFNKLQEKGIYKAMSEFDIFINYIEAYMTMKIRN

SEQ ID NO: 16 - protein, 178 aa, Homo sapiens
  MISC_FEATURE: Wild-type human IL-10; pre-protein with signal peptide
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 17 - protein, 178 aa, Artificial Sequence
  Synthetic construct
  MISC_FEATURE: 10_clone 5.1 (super-10)(N18Y/N92Q/T100D/R104W) with signal peptide
MHSSALLCCLVLLTGVRASPGQGTQSENSCTHFPGYLPNMLRDLRDAFSRVKTFFQMKDQLDNLLLKESLLEDFKGYLGCQALSEMIQFYLEEVMPQAENQDPDIKAHVQSLGENLKDLRLWLRRCHRFLPCENKSKAVEQVKNAFNKLQEKGIYKAMSEFDIFINYIEAYMTMKIRN

SEQ ID NO: 18 - protein, 178 aa, Artificial Sequence
  Synthetic construct
  MISC_FEATURE: 10_clone 5.2 (N18Y/N21H /N92Q/E96D/T100V R104W) with signal peptide
MHSSALLCCLVLLTGVRASPGQGTQSENSCTHFPGYLPHMLRDLRDAFSRVKTFFQMKDQLDNLLLKESLLEDFKGYLGCQALSEMIQFYLEEVMPQAENQDPDIKAHVQSLGDNLKVLRLWLRRCHRFLPCENKSKAVEQVKNAFNKLQEKGIYKAMSEFDIFINYIEAYMTMKIRN

SEQ ID NO: 19 - protein, 178 aa, Artificial Sequence
  Synthetic construct
  MISC_FEATURE: 10_clone 5.3 (N18Y/N21H/E96H T100V/R104W) with signal peptide
MHSSALLCCLVLLTGVRASPGQGTQSENSCTHFPGYLPHMLRDLRDAFSRVKTFFQMKDQLDNLLLKESLLEDFKGYLGCQALSEMIQFYLEEVMPQAENQDPDIKAHVNSLGHNLKVLRLWLRRCHRFLPCENKSKAVEQVKNAFNKLQEKGIYKAMSEFDIFINYIEAYMTMKIRN

SEQ ID NO: 20 - protein, 178 aa, Artificial Sequence
  Synthetic construct
  MISC_FEATURE: IL-10 variant BS63 (D25A); with signal peptide
MHSSALLCCLVLLTGVRASPGQGTQSENSCTHFPGNLPNMLRALRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 21 - protein, 178 aa, Artificial Sequence
  Synthetic construct
  MISC_FEATURE: IL-10 variant BS64 (N21A/R104A); with signal peptide
MHSSALLCCLVLLTGVRASPGQGTQSENSCTHFPGNLPAMLRDLRDAFSRVKTFFQMKDQLDNLLLKESLLEDFKGYLGCQALSEMIQFYLEEVMPQAENQDPDIKAHVNSLGENLKTLRLALRRCHRFLPCENKSKAVEQVKNAFNKLQEKGIYKAMSEFDIFINYIEAYMTMKIRN

SEQ ID NO: 22 - protein, 178 aa, Artificial Sequence
  Synthetic construct
  MISC_FEATURE: IL-10 variant BS65 (10-DE) (D25A/E96A); with signal peptide
MHSSALLCCLVLLTGVRASPGQGTQSENSCTHFPGNLPNMLRALRDAFSRVKTFFQMKDQLDNLLLKESLLEDFKGYLGCQALSEMIQFYLEEVMPQAENQDPDIKAHVNSLGANLKTLRLRLRRCHRFLPCENKSKAVEQVKNAFNKLQEKGIYKAMSEFDIFINYIEAYMTMKIRN

SEQ ID NO: 23 - protein, 178 aa, Artificial Sequence
  Synthetic construct
  MISC_FEATURE: IL-10 variant BS66 (N21A/D25A); with signal peptide
MHSSALLCCLVLLTGVRASPGQGTQSENSCTHFPGNLPAMLRALRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 24 - protein, 178 aa, Artificial Sequence
  Synthetic construct
  MISC_FEATURE: IL-10 variant BS68 (N21A/D25A/E96A); with signal peptide
MHSSALLCCLVLLTGVRASPGQGTQSENSCTHFPGNLPAMLRALRDAFSRVKTFFQMKDQLDNLLLKESLLEDFKGYLGCQALSEMIQFYLEEVMPQAENQDPDIKAHVNSLGANLKTLRLRLRRCHRFLPCENKSKAVEQVKNAFNKLQEKGIYKAMSEFDIFINYIEAYMTMKIRN

SEQ ID NO: 25 - protein, 178 aa, Artificial Sequence
  Synthetic construct
  MISC_FEATURE: IL-10 variant (N21A/D25A/ R104A); preprotein of SEQ ID NO: 10 with signal peptide
MHSSALLCCLVLLTGVRASPGQGTQSENSCTHFPGNLPAMLRALRDAFSRVKTFFQMKDQLDNLLLKESLLEDFKGYLGCQALSEMIQFYLEEVMPQAENQDPDIKAHVNSLGENLKTLRLALRRCHRFLPCENKSKAVEQVKNAFNKLQEKGIYKAMSEFDIFINYIEAYMTMKIRN

SEQ ID NO: 26 - protein, 178 aa, Artificial Sequence
  Synthetic construct
  MISC_FEATURE: IL-10 variant (D25K); preprotein of SEQ ID NO: 11 with signal peptide
MHSSALLCCLVLLTGVRASPGQGTQSENSCTHFPGNLPNMLRKLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 27 - protein, 178 aa, Artificial Sequence
  Synthetic construct
  MISC_FEATURE: IL-10 variant (E96K); preprotein of SEQ ID NO: 12 with signal peptide
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGKNLKTLRLRLRRCHRFLPCENKSKAVEQVKNAFNKLQEKGIYKAMSEFDIFINYIEAYMTMKIRN

SEQ ID NO: 28 - protein, 178 aa, Artificial Sequence
  Synthetic construct
  MISC_FEATURE: IL-10 variant (N18Y/D25A/N92Q/T100D/R104W); preprotein of SEQ ID NO: 13; with signal peptide
MHSSALLCCLVLLTGVRASPGQGTQSENSCTHFPGYLPNMLRALRDAFSRVKTFFQMKDQLDNLLLKESLLEDFKGYLGCQALSEMIQFYLEEVMPQAENQDPDIKAHVQSLGENLKDLRLWLRRCHRFLPCENKSKAVEQVKNAFNKLQEKGIYKAMSEFDIFINYIEAYMTMKIRN

SEQ ID NO: 29 - protein, 178 aa, Artificial Sequence
  Synthetic construct
  MISC_FEATURE: IL-10 variant (N18Y/D25K/N92Q/T100D/R104W); preprotein of SEQ ID NO: SEQ ID NO: 14; with signal peptide
MHSSALLCCLVLLTGVRASPGQGTQSENSCTHFPGYLPNMLRKLRDAFSRVKTFFQMKDQLDNLLLKESLLEDFKGYLGCQALSEMIQFYLEEVMPQAENQDPDIKAHVQSLGENLKDLRLWLRRCHRFLPCENKSKAVEQVKNAFNKLQEKGIYKAMSEFDIFINYIEAYMTMKIRN

SEQ ID NO: 30 - protein, 178 aa, Artificial Sequence
  Synthetic construct
  MISC_FEATURE: IL-10 variant (N18Y/D25A/N92Q/E96A/T100D/R104W); preprotein of SEQ ID NO: 15; with signal peptide
MHSSALLCCLVLLTGVRASPGQGTQSENSCTHFPGYLPNMLRALRDAFSRVKTFFQMKDQLDNLLLKESLLEDFKGYLGCQALSEMIQFYLEEVMPQAENQDPDIKAHVQSLGANLKDLRLWLRRCHRFLPCENKSKAVEQVKNAFNKLQEKGIYKAMSEFDIFINYIEAYMTMKIRN

SEQ ID NO: 31 - protein, 13 aa, Artificial Sequence
  Synthetic construct
  MISC_FEATURE: BAP tag
GLNDIFEAQKIEW

SEQ ID NO: 32 - protein, 160 aa, Artificial Sequence
  Synthetic construct
  MISC_FEATURE (14)..(14): Xaa is His, Ala, Asp, Glu, Ile, Lys, Leu, Met, Asn, Gln, Arg, Ser, Thr, Tyr, or Val
  MISC_FEATURE (18)..(18): Xaa is Asn, Phe, Ala, Asp, Glu, Leu, Val, Ser, Thr, Ile, Val, Met, or His
  MISC_FEATURE (21)..(21): Xaa is Asn, Ala, Arg, Gln, His, Lys, Ser, Val, Ile, Leu, Met, or Thr
  MISC_FEATURE (22)..(22): Xaa is Met, Ala, Val, Ile, Leu, Asn, or Gln
  MISC_FEATURE (24)..(24): Xaa is Arg, Glu, Asp, Asn, Gln, Ala, Ser, or Thr
  MISC_FEATURE (25)..(25): Xaa is Asp, Ala, Asn, His, Ile, Lys, Leu, or Val
  MISC_FEATURE (28)..(28): Xaa is Asp, Ala, Glu, Leu, Val, Ser, Thr, Ile, Val, Met, His, Lys, or Arg
  MISC_FEATURE (32)..(32): Xaa is Arg, Ala, Asp, Glu, Leu, Val, Ser, Thr, Ile, Val, Met, or His
  MISC_FEATURE (74)..(74): Xaa is Glu, Ala, Asp, Leu, Val, Ser, Thr, Ile, Val, Met, His, Lys, or Arg
  MISC_FEATURE (90)..(90): Xaa is His, Ala, Asp, Glu, Ile, Lys, Leu, Met, Asn, Gln, Arg, Ser, Thr, Tyr, or Val
  MISC_FEATURE (92)..(92): Xaa is Asn, Gln, Glu, His, Lys, Ser, Val, Ile, Leu, Met, Thr, or Ala
  MISC_FEATURE (93)..(93): Xaa is Ser, Glu, Ala, Arg, Asn, Asp, Gln, Glu, Ile, Leu, Lys, Met, or Val
  MISC_FEATURE (96)..(96): Xaa is Glu, Ala, Asn, Asp, Gln, His, Lys, or Ser
  MISC_FEATURE (100)..(100): Xaa is Thr, Val, Glu, Ala, Arg, Asn, Gln, Glu, Ile, Leu, Lys, Met, or Ser
  MISC_FEATURE (104)..(104): Xaa is Arg, Ala, Trp, Tyr, Phe, His, Asp, Glu, Asn, Gln, Ser, Thr, Ile, Leu, Val, or Met
SPGQGTQSENSCTXFPGXLPXXLXXLRXAFSXVKTFFQMKDQLDNLLLKESLLEDFKGYLGCQALSEMIQFYLXEVMPQAENQDPDIKAXVXXLGXNLKXLRLXLRRCHRFLPCENKSKAVEQVKNAFNKLQEKGIYKAMSEFDIFINYIEAYMTMKIRN